(19) United States
(12) Patent Application Publication
(10) Pub. No.: US 2019/0031759 A1
(43) Pub. Date: Jan. 31, 2019

(54) CHIMERIC ANTIGEN RECEPTORS AND METHODS OF THEIR USE

(71) Applicant: Technion Research & Development Foundation Limited, Haifa (IL)

(72) Inventors: Yoram REITER, Haifa (IL); Ravit OREN, Haifa (IL); Maya COHEN, Moshav HaBonim (IL)

(21) Appl. No.: 15/570,427

(22) PCT Filed: Apr. 30, 2015
(86) PCT No.: PCT/IL2015/050458

§ 371 Date: Oct. 30, 2017

(51) Int. Cl. C07K 16/28

(52) U.S. Cl. CPC C07K 16/2833; C07K 2319/03; C07K 2317/622; C07K 2317/92; C07K 2319/02

(57) ABSTRACT

Provided are chimeric antigen receptor (CAR) molecules comprising an extracellular domain comprising an antigen binding domain, a transmembrane domain, and an intracellular signaling domain, wherein an affinity of said binding domain to said antigen is characterized by a K_{D} higher than 150 nM. Also provided are isolated polynucleotides and nucleic acid constructs comprising same, cells transduced with same and methods of using same.

### FIELD AND BACKGROUND OF THE INVENTION

The present invention, in some embodiments thereof, relates to chimeric antigen receptors, cells transduced with same and, more particularly, but not exclusively, to methods of using same for treating various pathologies, such as cancer and autoimmune diseases, as well as infection diseases caused by viral or bacterial infections.

Adoptive transfer of antigen-specific T lymphocytes is an attractive form of immunotherapy for hematological malignancies and solid cancers. This approach, in which tumor reactive T cells undergo ex-vivo expansion and are then infused back to patients, has proven to be effective in metastatic melanoma patients. However, the widespread use of this approach is limited by the need to isolate antigen-specific T lymphocytes for individual patients.

To overcome this difficulty, the strategy of engineered T cells (CAR) has been developed, which involves genetic modifications based on 2 different approaches reviewed in Restifo, et al., 2012 (Adoptive immunotherapy for cancer: harnessing the T cell response. Nat Rev Immunol 12: 269-281).

In the first approach, tumor targeting by T cells is achieved using a cloned αβ TCR which is introduced into the cells and enables specific MHC-restricted targeting of tumor cells. This approach has been proven effective in clinical trials in melanoma) and several groups are currently working to improve the expression of the exogenous TCR on the surface of T cells.

The second strategy involves redirection of T cells based on antibody variable fragments (Fv). The availability of anti-tumor antibodies targeting a variety of tumors prompted the idea of incorporating the recognition domain of these antibodies in the form of single chain Fv (scFv) domain in a chimeric receptor construct (13). This chimeric antibody-based or antigen receptor (CAR) is based on linking the recognition elements of an antibody to signaling moieties for T cell activation, thereby redirecting T cells to a desired antigen in an either non-MHC restricted or an MHC restricted manner.

In the non-MHC restricted manner, T cells are redirected independently of MHC and proved to be effective against tumor cells that lost their HLA expression due to tumor escape mechanisms (14). After several studies in the last decade that demonstrated the effectiveness of this approach in inducing tumor regression in mouse models, this approach is currently being evaluated in clinical trials (15-17). Furthermore, this strategy has shown dramatic responses in a pilot clinical trial in chronic lymphocytic leukemia (CLL) patients treated with CD19-specific CAR T cells (18). Targeting tumor-associated antigens with engineered T cells that express a specific CAR is an ideal approach combining the unique expression pattern of tumor associated antigens (TAAs) with potent killing mediated by the engineered effector T cell (19).

For an MHC restricted manner, antibodies which recognize specific peptide/MHC complexes (termed “TCR like antibodies, or TCRLs) can be used (26). The present inventors and others have isolated antibodies which recognize HLA-A2 complexes bearing peptides derived from tumor and viral antigens by means of phage display and hybridoma strategies (27-30). These TCR-like antibodies, which exhibit both binding properties and kinetics of antibodies (e.g., high affinity), while mimicking the specificity of TCRs, are being used as a novel research tool to study antigen presentation and immunotherapy targets.

Wilm's tumor suppressor gene 1 (WT1) is one of the most important TAAs classified by the National Cancer Institute (NCI). WT1, a zinc finger transcription factor, is highly expressed in many solid cancers and leukemia cells, but not in normal tissues (including hematopoetic progenitor and stem cells). Several studies have suggested that WT1 may have an essential role in leukemogenesis/tumorgenesis, and is required to maintain the transformed phenotype/function; therefore, tumor escape from immune surveillance as a result of down-regulation of WT1 expression is unlikely to occur, marking WT1 as an attractive and important target for immunotherapy.

The WT1_{Db126} (RMFPNAPYL; SEQ ID NO:1) peptide was identified by screening the WT1 amino acid sequence (GenBank Accession NO. EAW68225, Wilms tumor 1, isoform CRA_f; SEQ ID NO:3) for 9-mer peptides that include major anchor motifs for binding to HLA-A2 (24). In-vitro immunization elicits WT1 peptide-specific CTLs which mediate lysis of WT1-expressing tumor cells, indicating that this peptide constitutes a highly immunogenic epitope. The αβ genes of a TCR which recognizes HLA-A2-WT1_{Db126} complexes were isolated using an allogeneic repertoire, and were introduced by retroviral transduction into human T cells (7). This TCR exhibited efficient and specific reactivity with HLA-A2-WT1_{Db126} complexes, enabling specific cytolytic reactivity by CTLs expressing the TCR toward target cells (25).

CARs have been successfully used in the treatment of various leukemias, and they are currently being used in clinical trials also for various solid tumors, however, their use is limited by the number of cancer cells which are not recognized by T cells, mainly due to limited availability of tumor-specific T cells and deficiencies in antigen processing or major histocompatibility complex (MHC) expression of cancer cells. Additional background art includes Chmielewski et al., 2013 (Antigen-specific T-cell activation independently of the MHC: chimeric antigen receptor-redirected T cells. Frontiers in Immunology, vol. 4, article 371), U.S. Patent Application No. 20130287748.

### SUMMARY OF THE INVENTION

According to an aspect of some embodiments of the present invention there is provided a chimeric antigen receptor (CAR) molecule comprising an extracellular domain comprising an antigen binding domain, a transmembrane domain, and an intracellular signaling domain, wherein an affinity of the binding domain to the antigen is characterized by a K_{D} higher than 150 nM.

According to an aspect of some embodiments of the present invention there is provided an isolated polynucleotide comprising a nucleic acid sequence encoding the molecule of some embodiments of the invention.

According to an aspect of some embodiments of the present invention there is provided a nucleic acid construct comprising an isolated polynucleotide comprising a nucleic acid sequence encoding the molecule of some embodiments of the invention and a cis-acting regulatory element for directing transcription of the isolated polynucleotide in a host cell.

According to an aspect of some embodiments of the present invention there is provided an isolated cell comprising the polynucleotide of claim of some embodiments of the invention or the nucleic acid construct of some embodiments of the invention.

According to an aspect of some embodiments of the present invention there is provided a pharmaceutical composition comprising the CAR molecule of some embodiments of the invention, the isolated polynucleotide of some embodiments of the invention, the nucleic acid construct of some embodiments of the invention or the cell of some embodiments of the invention and a pharmaceutically acceptable carrier.

According to an aspect of some embodiments of the present invention there is provided an in vitro method of generating a medicament for treating a pathology in a subject in need thereof, comprising:

(a) obtaining T cells or natural killer (NK) cells,
(b) transducing the T cells or the natural killer with the nucleic acid construct of some embodiments of the invention, wherein binding of the molecule to the antigen elicits a therapeutic response by the T cells or the natural killer of the subject, thereby generating the medicament for treating the pathology.

According to an aspect of some embodiments of the present invention there is provided a method of treating a pathology in a subject in need thereof, comprising administering the medicament resultant of claim 36 in the subject, thereby treating the pathology in the subject.

According to some embodiments of the invention, the antigen is an MHC restricted antigen.

According to some embodiments of the invention, the MHC restricted antigen comprises an MHC class I restricted antigen.

According to some embodiments of the invention, the MHC restricted antigen comprises an MHC class II restricted antigen.

According to some embodiments of the invention, the antigen is a non-MHC restricted antigen.

According to some embodiments of the invention, the MHC-restricted antigen is a tumor associated antigen.

According to some embodiments of the invention, the MHC-restricted antigen is a viral antigen.

According to some embodiments of the invention, the MHC-restricted antigen is an autoimmune antigen.

According to some embodiments of the invention, the tumor associated antigen comprises the WT1 protein.

According to some embodiments of the invention, the tumor associated antigen comprises the tyrosinase protein.

According to some embodiments of the invention, the MHC-restricted tumor associated antigen is the WT1_{Db126} peptide set forth in SEQ ID NO:1.

According to some embodiments of the invention, the wherein the antigen binding domain comprises CDRs which are derived from an antibody.

According to some embodiments of the invention, the wherein the antigen binding domain comprises CDRs which are derived from a T cell receptor (TCR).

According to some embodiments of the invention, the wherein the antigen binding domain comprises a single chain Fv (scFv) molecule.

According to some embodiments of the invention, the tumor associated antigen is selected from the group consisting of: Uroplakin II (UPKII), Uroplakin Ia (UPK1A), prostate specific antigen (NPSA), prostate specific membrane antigen (PSCA), prostate acid phosphatase (ACPP), BA-46, MFGE8 milk fat globule-EGF factor 8 protein [lactadherin], Mucin 1 (MUC1), premelanosome protein (PMEL, Gp100), melan-A (MLANA, MART1), telomerase reverse transcriptase (TERT), TAX, NY-ESO cancer/testis antigen 1B (CTAG1B), Melanoma antigen family A1 (MAGEA1), Melanoma antigen family A3 (MAGEA3, MAGE-A3), erb-b2 receptor tyrosine kinase 2 (ERBB2, HER2), Beta-catenine (CTNNB1), Tyrosinase (TYR), and Bcr-abl, caspase 8 (CASP8).

According to some embodiments of the invention, the viral antigen is derived from a virus selected from the group consisting of: human immunodeficiency virus (HIV), influenza, Cytomegalovirus (CMV), T-cell leukaemia virus type 1 (TAX), hepatitis C virus (HCV) and hepatitis B virus (HBV).

According to some embodiments of the invention, the autoimmune antigen is associated with type 1 diabetes, the autoimmune antigen is derived from a polypeptide selected from the group consisting of: preproinsulin, proinsulin, Glutamic acid decarboxylase (GAD), Insulinoma Associated protein 2 (IA-2), IA-20 (phogrin), Islet-specific Glucose-6-phosphatase catalytic subunit-Related Protein (IGRP), chromogranin A, Zinc Transporter 8 (ZnT8), Heat Shock Protein-60 (HSP-60), and Heat Shock Protein-70 (HSP-70).

According to some embodiments of the invention, the autoimmune antigen is associated with multiple sclerosis the autoimmune antigen is derived from a polypeptide selected from the group consisting of: myelin oligodendrocyte glycoprotein (MOG), myelin basic protein (MBP), and proteolipid protein (PLP1).

According to some embodiments of the invention, the autoimmune antigen is associated with rheumatoid arthritis the autoimmune antigen is derived from a polypeptide selected from the group consisting of: Collagen II (COL2A1), Matrix metalloproteinase-1 (MMP1), Aggrecan Core Protein Precursor (ACAN), Matrix Metalloproteinase-16 (MMP16), Tenascin (TNXB) and Heterogeneous Nuclear Ribonucleoprotein A2 (HNRNPA2B1).

According to some embodiments of the invention, the autoimmune antigen is associated with celiac the autoimmune antigen is derived from a polypeptide selected from the group consisting of: alpha Gliadin, gamma Gliadin and Heat shock 20. According to some embodiments of the invention, the autoimmune antigen is associated with stroke the autoimmune antigen is derived from a polypeptide selected from the group consisting of: myelin basic protein, neurofilament and NR2A/2B subtype of the N-methyl-D-aspartate receptor.

According to some embodiments of the invention, the non-MHC restricted antigen is selected from the group consisting of a-Folate receptor, CAIX, CD19, CD20, CD22, CD30, CD33, CD44v7/8, CEA, EGP-2, EGP-40, erb-B2, erb-B 2,3,4, FBP, Fetal acetylcholine receptor, GD2, GD3, Her2/neu, IL-13R-a2, KDR, k-light chain, LeY, L1 cell adhesion molecule, MAGE-A1, Mesothelin, Murine CMV infected cells, MUC1, NKG2D ligands, Oncofetal antigen (h5T4), PSCA, PSMA, TAA targeted by mAb IgE, TAG-72, and VEGF-R2.

According to some embodiments of the invention, the K_{D} is higher than 400 nM.

According to some embodiments of the invention, the K_{D} is selected from a range of about 200 nM (nanomolar) to about 5 μM (micromolar).

According to some embodiments of the invention, the intracellular signaling domain comprises the polypeptide selected from the group consisting of: CD3ζ (CD247, CD3z), CD28, 4-1BB (CD137), ICOS, and OX40.

According to some embodiments of the invention, the cell is a T cell or natural killer (NK) cell.

According to some embodiments of the invention, the T cell is obtained from peripheral blood mononuclear cells (PBMCs).

According to some embodiments of the invention, the T cell comprises a Treg (T regulatory cell).

According to some embodiments of the invention, the T cell comprises a CD4 T cell.

According to some embodiments of the invention, the T cell comprises a CD8 T cell.

According to some embodiments of the invention, the T cell is a cytotoxic T cell.

According to some embodiments of the invention, the T cells or the natural killer are autologous to the subject.

According to some embodiments of the invention, the T cells or the natural killer are semi-autologous to the subject.

According to some embodiments of the invention, the T cells or the natural killer are non-autologous to the subject.

According to some embodiments of the invention, the T cells are obtained from peripheral blood mononuclear cells (PBMCs) of the subject in need thereof.

According to some embodiments of the invention, the pathology is a solid tumor.

According to some embodiments of the invention, the pathology is a viral infection.

According to some embodiments of the invention, the pathology is an autoimmune disease.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-C show binding properties of F2 and F3 anti-HLA-A2/WT1_{Db126} TCR-like recombinant antibodies. FIG. 1A—ELISA of anti-HLA-A2-WT1_{Db126} soluble purified Fabs with immobilized HLA-A2-WT1_{Db126} complexes versus control complexes displaying control peptide. Anti-HLA mAb W6/32 was used to determine the correct folding and stability of the bound complexes during the binding assay. FIG. 1B—Flow cytometry analysis of the binding of F2 and F3 Fabs to T2 cells loaded with WT1_{Db126} peptide or to the control peptides 209m (SEQ ID NO:12), Gag (SEQ ID NO:14) and mdm2 (SEQ ID NO:15). FIG. 1C—Surface plasmon resonance (SPR) analysis of F2 and F3 Fabs for affinity to HLA-A2-WT1_{Db126} complexes which was determined as 400 nM and 30 nM, respectively.

FIG. 2 shows reactivity of F2 anti-HLA-A2-WT1_{Db126} TCR-like Fab with tumor cell lines. Detection of HLA-A2-WT1_{Db126} complexes on the surface of HLA-A2⁺ WT1⁺ cell lines: 501A and Skmel5 melanoma cell lines; Loucy ALL cell line; CCRF-SB (data not shown) and DG75 lymphoma cell line; MDA-MB-231 human breast carcinoma; SW620, Caco-2 and colo-205 human colon cancer; Panc-1 human pancreatic carcinoma; Hep-G2 (data not shown) liver hepatocellular carcinoma; UMUC3 human bladder transitional cell carcinoma, and BV173 leukemia cell line (ALL). Cells were incubated with anti-HLA-A2-WT1_{Db126} TCR-like Fabs, followed by incubation with PE-labeled anti-human Ab. HLA-A2-negative (WT1-positive) A431 epidermoid carcinoma cell line and WT1-positive (HLA-A2-negative). Fibroblast cells (Fibs) were used as controls.

FIG. 3 shows expression of TCR like chimeric receptor on transduced Jurkat cells. Jurkat cells were transduced with retroviral vector encoding the 400 nM F2 anti-HLA-A2-WT1_{Db126} chimeric receptor construct; 96 hours after transduction, cells were stained with PE-labeled-HLA-A2 tetramers presenting the WT1_{Db126} specific peptide or control peptides and were analysed by flow cytometry. GFP expression represents positive transduced Jurkat cells.

FIGS. 4A-B depict expression of TCR-like chimeric receptors on transduced human T cells. HLA-A2+(FIG. 4A) and HLA-A2⁻ (FIG. 4B) activated primary T cells were transduced with either vector encoding the F2 or F3 TCR like chimeric receptors; 48 hours after transduction the cells were stained with anti-CD3 and anti-CD8 Abs and with the fluorescently labelled HLA-A2-WT1_{Db126} tetramer. Cells were gated on the CD3+ T cell populations.

FIG. 5 depicts expression of F2 TCR like chimeric receptor and WT1_{Db126} αβTCR construct (marked “TCR” in FIG. 5) on transduced human T cells. HLA-A2⁺ activated primary T cells were transduced with either a vector encoding the αβTCR or F2 chimeric receptor; 48 hours after transduction the cells were stained with anti-CD3, CD8, Vβ2.1 (an antibody directed against the variable chain of the β chain of the αβTCR) antibodies or HLA-A2-WT1_{Db126} tetramer. Cells were gated on the CD3+ T cell populations.

FIGS. 6A-C depict response of CARs transduced T cells to antigen-specific stimulation. FIG. 6A—FACS analysis of freshly transduced T cells with either 400 nM F2 TCR-like chimeric receptor (left panels) or WT1_{Db126} αβTCR (right panels). Transduced cells were stimulated for 18 hours with T2 cells loaded with 100 μM either relevant (WT1_{Db126}; SEQ ID NO:1) or control (WT1₂₃₅; SEQ ID NO:7) peptide. Cells were stained for CD8, and then fixed, permeabilized and stained with anti-IFN-gamma and anti-IL2 Abs followed by flow cytometry analysis. Shown is the percentage of CD8⁺ T expressing IFN-gamma and IL-2. The percentage of positive transduced CD8+ cells was 30% and 42% for TCR and F2, respectively. FIG. 6B—ELISA assay for IFN-gamma release. Transduced T cells were stimulated for 18 hours with T2 cells loaded with decreasing dilutions of relevant (WT_{Db126}) peptide (100 μM to 0.1 μM) or control (Gp100-280) peptide. Interferon (IFN)-gamma release was determined by ELISA. The percentage of positive transduced CD8⁺ cells was 57% and 41% for TCR and F2, respectively. The percentage of positive transduced CD4⁺ cells was 48% and 30% for TCR and F2, respectively. This is a representative of 3 independent experiments showing similar results. FIG. 6C—Flow cytometry for CD107a expression on CD8+ T cell. Transduced T cells were stimulated for 4 hours with T2 cells loaded with decreasing dilutions of relevant (WT_{Db126}) peptide (100 μM to 0.1 μM) or control (Gp100-280) peptide. Percent of CD107a was determined by Flow cytometry. This is a representative of 3 independent experiments showing similar results.

FIGS. 7A-C depict antigen-specific cytotoxic activity of CARs transduced T cells. FIG. 7A—Transduced T cells were cultured in a 4-hour assay with ³⁵S-labeled T2 cells loaded with either 100 μM relevant (WT1_{Db126}) or control (Gp100-280) peptide at the stated E:T ratios. This is a representative of 3 independent experiments showing similar results. FIG. 7B—Transduced T cells were cultured in a 4-hour assay with ³⁵S-labeled T2 cells, loaded with decreasing dilution of WT1_{Db126} peptide (100 μM to 0.1 μM) or control peptideGp100-280 (100 μm), at an E:T ratio of 5:1. This is a representative of 3 independent experiments showing similar results. In both assays the amount of positive transduced cells added was normalized respectively to the highest percent of transduced cell. FIG. 7C—Transduced T cells were cultured in a 4-hour assay with ³⁵S-labeled 501A Melanoma and Breast MDA231 (HLA-A2+WT1_{Db126+}) or control A431 (HLA-A2-WT1_{Db126+}) at the stated E:T ratios. In all assays, the percentage of positive transduced CD8⁺ cells was 57% and 41% for TCR and F2, respectively. The percentage of positive transduced CD4⁺ cells was 48% and 30% for TCR and F2. A and C represent a typical experiment out of 3 independent repetitions.

FIGS. 8A-D depict antigen-specific cytotoxic activity of CD8 and CD4 transduced T cells. Purified CD4 and CD8 subpopulations were obtained by incubating the T cells with anti-CD4, anti-CD8 and anti-mouse IgG-coated magnetic beads. FIG. 8A—Transduced purified CD8 T cells were cultured in a 4-hour assay with ³⁵S-labeled T2 cells, loaded with decreasing dilution of WT1_{Db126} peptide (100 μM to 0.1 μM) or control peptideGp100-280 (100 μm), at an E:T ratio of 5:1. FIG. 8B—Transduced purified CD8 T cells were cultured in a 4-hour assay with ³⁵S-labeled T2 cells loaded with either 100 μM relevant (WT1_{Db126}) or control (Gp100-280) peptide at the stated E:T ratios. FIG. 8C—Transduced purified CD4 T cells were cultured in a 4-hour assay with ³⁵S-labeled T2 cells, loaded with decreasing dilution of WT1_{Db126} peptide (100 μM to 0.1 μM) or control peptideGp100-280 (100 μm), at an E:T ratio of 5:1. FIG. 8D—Transduced purified CD4 T cells were cultured in a 4-hour assay with ³⁵S-labeled T2 cells loaded with either 100 μM relevant (WT1_{Db126}) or control (Gp100-280) peptide at the stated E:T ratios. This is a representative of 3 independent experiments showing similar results. In all assays the amount of positive transduced CD8 and CD4 F2 and TCR CD8 and CD4 T cells added was normalized respectively to 60% which was the highest percent of transduce CD8 TCR transduced cell.

FIGS. 9A-B provide the sequences of the F2 T body (CAR) VL-VH in pBULLET. FIG. 9A—amino acid sequence; FIG. 9B—nucleic acid sequence. Color index: Red=Leader (SEQ ID NOs:20-21); Green=VL (SEQ ID NOs:16 and 18); Dark blue=Linker (SEQ ID NOs:2 and 23); Orange=VH (SEQ ID NOs:17 and 19); Light blue=CD28 (SEQ ID NOs:24-25); Purple=“gamma” (FcγRI γ) (SEQ ID NOs:26-27).

FIGS. 10A-B provide the sequences of the F3 T body (CAR) VL-VH in pBULLET. FIG. 9A—amino acid sequence; FIG. 9B—nucleic acid sequence. Color index: Red=Leader (SEQ ID NOs:20-21); Green=VL (SEQ ID NOs: 28 and 30); Dark blue=Linker (SEQ ID NOs: 2 and 23); Orange=VH (SEQ ID NOs:29 and 31); Light blue=CD28 (SEQ ID NOs:24-25); Purple=“gamma” (FcγRI γ) (SEQ ID NOs:26-27).

### DESCRIPTION OF SPECIFIC EMBODIMENTS OF THE INVENTION

The present invention, in some embodiments thereof, relates to chimeric antigen receptor molecules, nucleic acid constructs comprising same, cells transformed with same and, more particularly, but not exclusively, to methods of using same for generating a medicament for treating viral infections, bacterial infections, cancer and autoimmune diseases. Thus, the present invention relates to a strategy of adoptive cell transfer of cells (e.g., T cells or NK cells) transduced to express a chimeric antigen receptor (CAR).

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

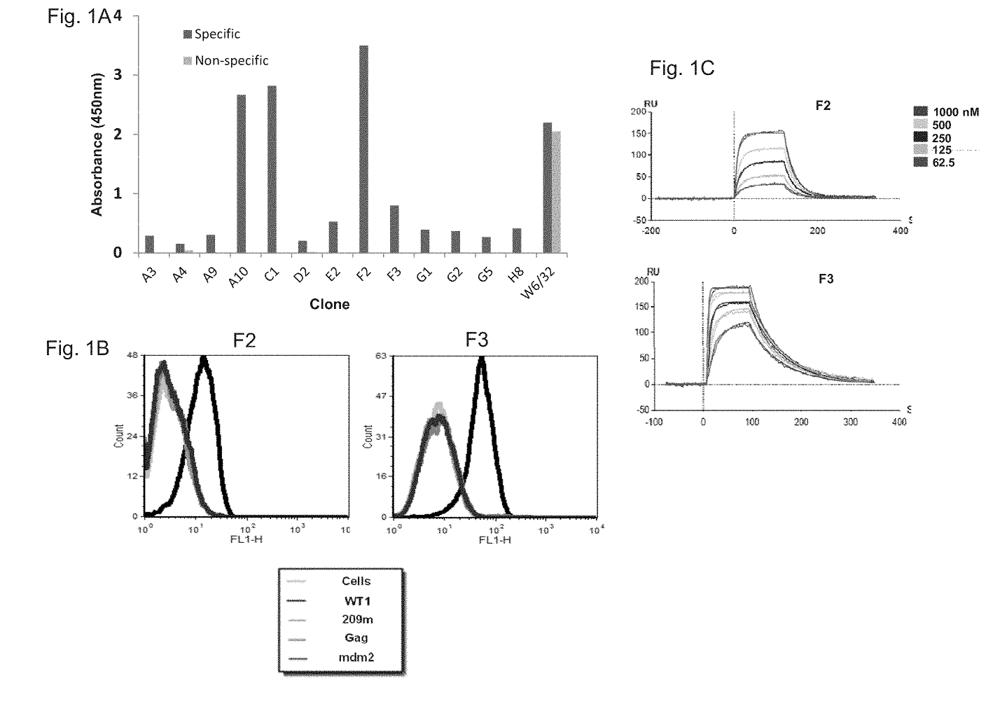

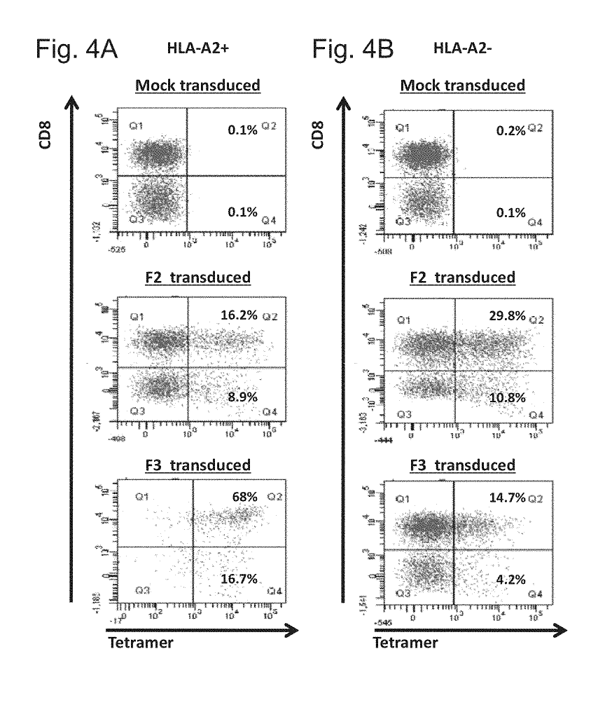

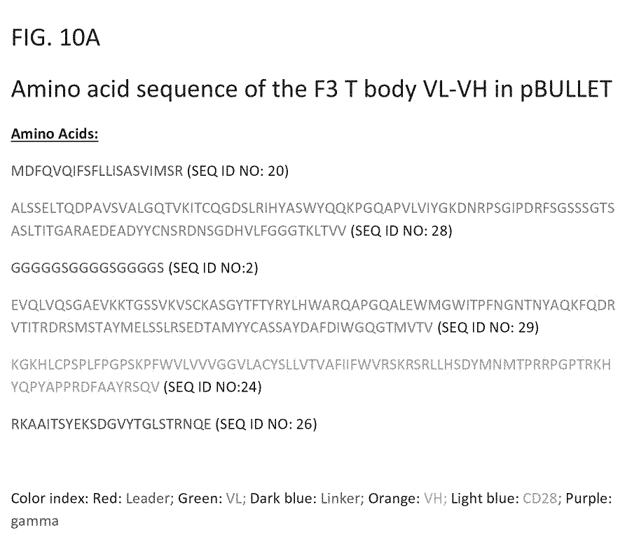

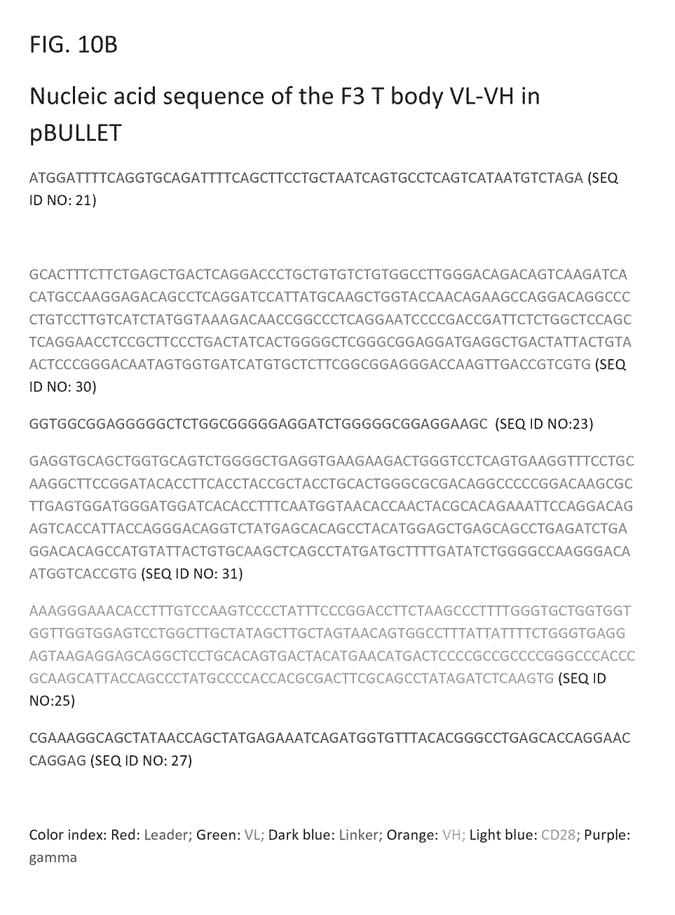

The present study investigates how the differences in affinity and avidity of an αβ TCR versus a TCR-like antibody-based CAR play a functional role in engineered T cells that carry these recognition moieties. The present inventors have isolated TCR-like Fab antibodies which recognize the HLA-A2 molecule bearing the WT1-derived peptide (WT1_{Db126}; RMFPNAPYL) with different affinities (400 nM and 30 nM as determined by Surface plasmon resonance (SPR) analysis (FIG. 1C)). CARs in which the antigen binding domain includes the VH and VL sequences of a TCR-like receptor (e.g., the F2 and F3 antibodies) were generated based on the isolated Fabs and used to redirect T cells toward HLA-A2-WT1_{Db126} (FIGS. 1-3 and 9-10 and Examples 1 and 2 of the Examples section which follows). In comparison, the present inventors used engineered T cells that carry αβ TCR genes targeting the same WT1-specific epitope. These redirected T cells exhibited efficient and specific reactivity with HLA-A2-WT1_{Db126} complexes. T cells transduced with CAR molecules which include either the F2 or the F3 TCRL were shown to efficiently express the F2 and F3 TCRLs (FIG. 4A), however, the viability of the T cells transduced with the high affinity (30 nM) F3 TCRL was very low. Without being bound by any theory, these results suggest that the elevated affinity of the 30 nM F3 TCR-like CAR, in combination with the high avidity of the receptor present on the T cell surface, may lead to some loss of specificity and consequently to decreased cell survival.

Using these TCR-like antibody-based CARs and the recombinant αβTCR based CAR, the present inventors directly compared, for the first time, 2 different approaches for redirecting T cells in order to enhance the understanding of the influence and relationships of affinity and avidity on the biological functions of T cells being redirected by either cloned αβ TCRs or TCR-like antibodies based CARs.

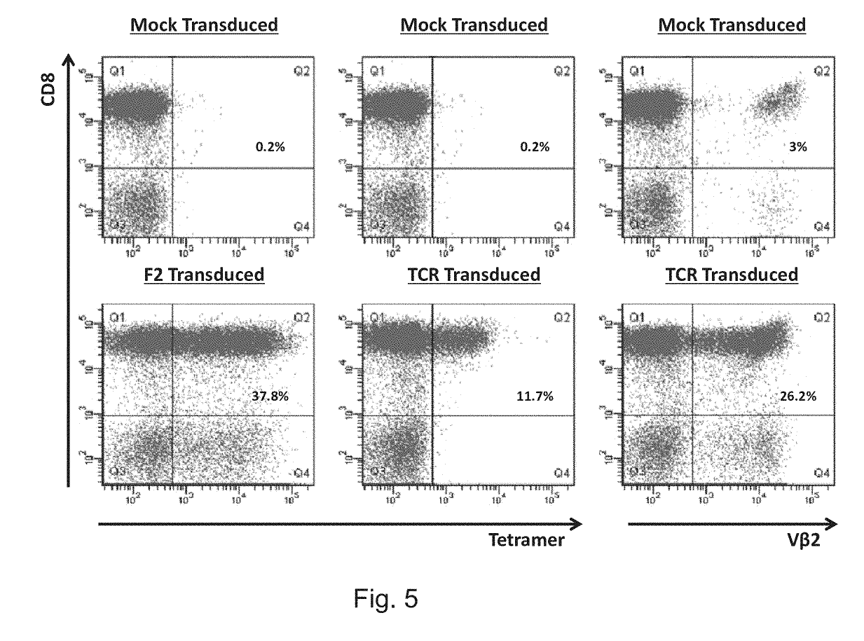

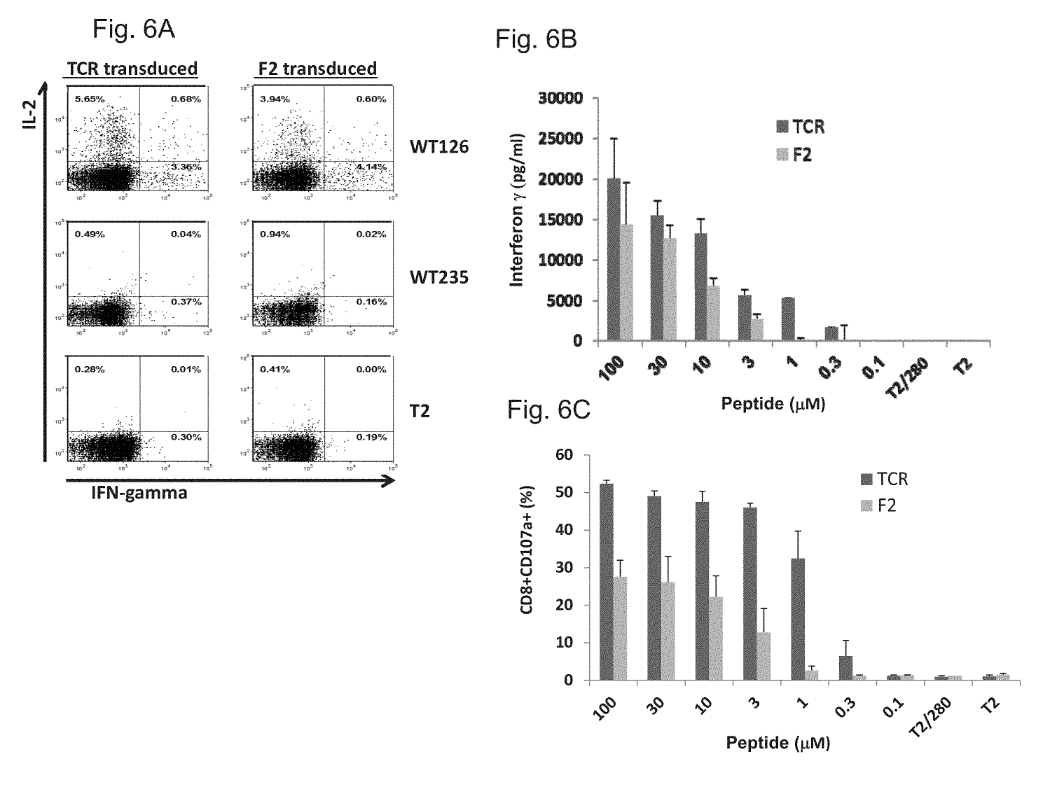

Example 3 of the Examples section which follows demonstrates efficient transductions of HLA-A2+ T cells transduced with either the αβTCR CAR construct or the F2 TCR like CAR construct (FIG. 5). Moreover, Example 4 (FIG. 6B) demonstrates determination of T cells avidity using interferon release assays and shows that the avidity of the T cells transduced with the CAR of WT1 αβTCR is between 3-10 μM, e.g., about 5 μM, and the avidity of the T cells transduced with the CAR of F2 TCRL is about 10 μM.

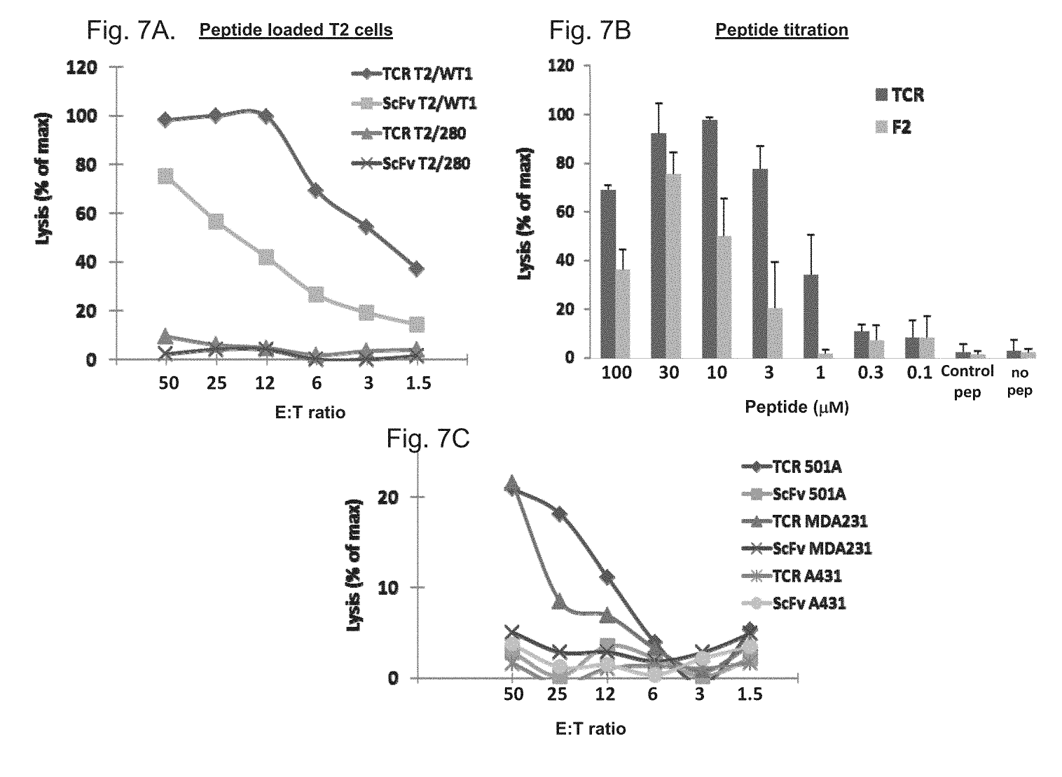

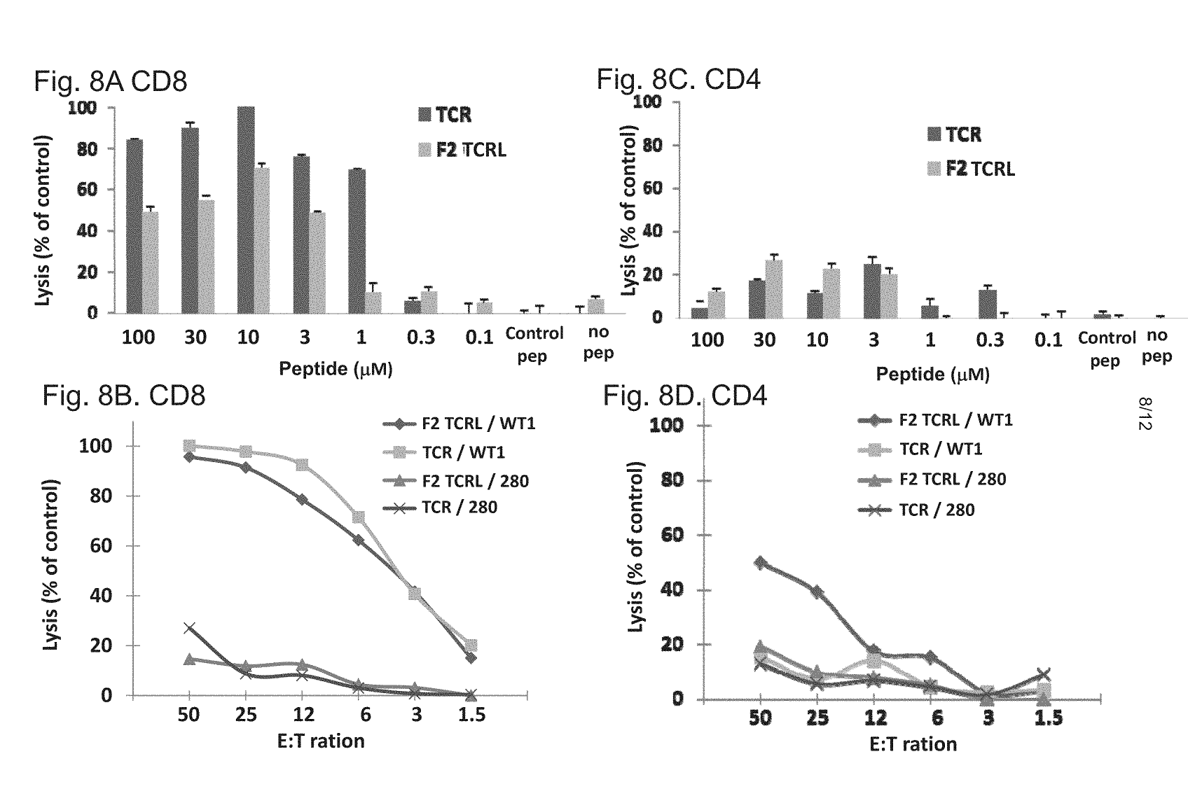

In addition, T cells transduced with the αβTCR showed greater specific cytolytic activity than T cells transduced with the F2 TCR-like Ab CAR (FIG. 7A and Example 5 of the Examples section which follows). Moreover, the sensitivity of the αβTCR-transduced T cells was indeed greater compared with the TCR-like Ab-transduced cells as the αβTCR cells were more efficient in mediating killing of WT1_{Db126} loaded T2 cells at low peptide concentrations (FIG. 7B and Example 5 of the Examples section which follows). These results further correspond to the killing sensitivity of the αβTCR transduced T cells (FIG. 7C and Example 5). Moreover, αβTCR CAR-transduced CD8+ T cells were more active compared to F2 TCRL CAR-transuded cells similarly with what was observed with the intact whole T cell population (FIGS. 8A-D; Example 6). Altogether, these results support the findings that an upper affinity threshold for TCR-based recognition is required to mediate effective and optimal functional activity in killing of target cells. In this context, high affinity CARs which can be composed of TCRL antibody variable fragments, or affinity enhanced αβTCRs are less suitable and attractive than native αβTCR or TCRL antibodies with moderate affinity (e.g., having a K_{D} higher than 150 nM) for the design of CARs.

The present inventors demonstrate herein that the combination of high affinity and avidity of a TCR-like antibody displayed on the surface of the engineered T cells has dramatic effects on the specificity and function of these T cells compared to engineered T cells carrying a low affinity αβ TCRs. The present inventors thus present evidence for a TCR-based affinity threshold which limits the maximal T-cell effective function and suggests that rational design of improved TCRs or TCR-like antibodies for T-cell redirection may need to be optimized up to a given affinity threshold in order to achieve optimal T cell function without risking cross reactivity. The comparison presented herein enables better understanding of limits and thresholds in T-cell recognition using these 2 different approaches, and clarifies the optimal recognition properties required for most effective and specific T cell retargeting toward tumor cells.

Thus, according to an aspect of an embodiment of the invention there is provided a chimeric antigen receptor (CAR) molecule comprising an extracellular domain comprising an antigen binding domain, a transmembrane domain, and an intracellular signaling domain, wherein an affinity of the binding domain to the antigen is characterized by a K_{D} higher than 150 nM.

As used herein the phrase “chimeric antigen receptor (CAR)” refers to a recombinant or synthetic molecule which combines antibody-based specificity for a desired antigen with a T cell receptor-activating intracellular domain to generate a chimeric protein that exhibits cellular immune activity to the specific antigen.

The choice of antigen binding domain (or moiety) depends upon the type and number of ligands that define the surface of a target cell. For example, the antigen binding domain may be chosen to recognize a ligand that acts as a cell surface marker on target cells associated with a particular disease state. Thus examples of cell surface markers that may act as ligands for the antigen binding domain in the CAR molecule of the invention include those associated with viral, bacterial and parasitic infections, autoimmune disease and cancer cells.

The term “antigen” or “Ag” as used herein is defined as a molecule that provokes an immune response. The skilled artisan will understand that any macromolecule, including virtually all proteins or peptides, as well as carbohydrates, lipids and DNA can serve as an antigen.

According to some embodiments of the invention, the antigen is a tumor antigen (e.g., tumor specific antigen or a tumor associated antigen), a viral protein antigen, a bacterial protein antigen, or an autoimmune associated antigen (e.g., a “self” antigen).

According to some embodiments of the invention, the antigen is presented by an MHC molecule.

According to some embodiments of the invention, the antigen is not presented on MHC molecule.

According to some embodiments of the invention, the antigen to which the CAR molecule binds is a protein-derived antigen.

Thus, the extracellular domain of the CAR molecule comprises an antigen binding domain, wherein an affinity of the antigen binding domain to the antigen is characterized by a K_{D} which is higher than 150 nM. It should be noted that an affinity characterized by a K_{D} higher than 150 nM is considered a relatively low affinity as compared to an affinity between an antibody and its antigen.

The affinity of the antigen binding domain to its antigen is determined using the soluble molecules from which the CDRs of the antigen binding domain of the CAR were derived.

As used herein the term “K_{D}” refers to the equilibrium dissociation constant between the antigen binding domain and its respective antigen.

According to some embodiments of the invention, such affinity to the antigen (which is characterized by a K_{D} higher than 150 nM) by the CAR's antigen binding domain enables T cell activity or an NK cell (natural killer cell) activity, e.g., effector cytotoxic function, regulatory function, and/or NK target cell killing function.

Non-limiting examples of assays which can be used to determine T cell or NK activity include cytotoxic activity, cytokine release, expression of activation markers (e.g., CD69, CD25, granulation markers, CD107a), function of Treg (suppression of T cell effector function), and/or function of NK cells (e.g., target cell killing).

Similar measurements which determine T cell function relate to the functional avidity of CAR-expressing T cell including affinity threshold that enables maximal CD8 and CD4 T effector T cell function.

It should be noted that the affinity of the antigen binding domain to its antigen can be quantified using known methods. For example, when using Surface Plasmon Resonance (SPR) [described in Scarano S, Mascini M, Turner A P, Minunni M. Surface plasmon resonance imaging for affinity-based biosensors. Biosens Bioelectron. 2010, 25: 957-66, which is fully incorporated herein by reference in its entirety] a soluble molecule such as an antibody [e.g., a TCRL antibody, or a cloned extracellular domain of the αβTCR which is stabilized by various means (e.g., by introducing disulphide bonds, or linkers between the units)] is tested for its binding to the antigen, and the affinity is determined by calculating the K_{D} dissociation constant. It should be noted that a higher K_{D} reflects a lower affinity.

According to some embodiments of the invention, the affinity of the antigen binding domain to the antigen is characterized by a K_{D} which is higher than about 150 nM, e.g., higher than about 200 nM, e.g., higher than about 250 nM, e.g., higher than about 300 nM, e.g., higher than about 350 nM, e.g., higher than about 400 nM, e.g., higher than about 450 nM, e.g., higher than about 500 nM, e.g., higher than about 550 nM, e.g., higher than about 600 nM, e.g., higher than about 650 nM, e.g., higher than about 700 nM, e.g., higher than about 750 nM, e.g., higher than about 800 nM, e.g., higher than about 850 nM, e.g., higher than about 900 nM, e.g., higher than about 950 nM, e.g., higher than about 1000 nM, e.g., higher than about 1100 nM, e.g., higher than about 1200 nM, e.g., higher than about 1300 nM, e.g., higher than about 1400 nM, e.g., higher than about 1500 nM, e.g., higher than about 2000 nM, e.g., higher than about 2500 nM, e.g., higher than about 3000 nM, e.g., higher than about 3500 nM, e.g., higher than about 4000 nM, e.g., higher than about 4500 nM, e.g., higher than about 4800 nM, e.g., about 5000 nM (i.e., 5 μM).

According to some embodiments of the invention, the affinity of the antigen binding domain to the antigen is characterized by a K_{D} which is between about 200 nM (nanomolar) to about 5 μM (micromolar), e.g., between about 250 nM to about 5 μM, e.g., between about 300 nM to about 5 μM, e.g., between about 350 nM to about 5 μM, e.g., between about 400 nM to about 5 μM, e.g., between about 450 nM to about 5 μM, e.g., between about 550 nM to about 5 μM, e.g., between about 600 nM to about 5 μM, e.g., between about 650 nM to about 5 μM, e.g., between about 700 nM to about 5 μM, e.g., between about 750 nM to about 5 μM, e.g., between about 800 nM to about 5 μM, e.g., between about 1000 nM to about 5 μM, e.g., between about 1200 nM to about 5 μM, e.g., between about 1400 nM to about 5 μM, e.g., between about 1500 nM to about 5 μM, e.g., between about 2 μM to about 5 μM, e.g., between about 2.5 μM to about 5 μM, e.g., between about 3 μM to about 5 μM, e.g., between about 4 μM to about 5 μM; e.g., between about 200 nM (nanomolar) to about 4.5 μM (micromolar), e.g., between about 250 nM to about 4 μM, e.g., between about 300 nM to about 4 μM, e.g., between about 350 nM to about 4 μM, e.g., between about 400 nM to about 4 μM, e.g., between about 450 nM to about 4 μM, e.g., between about 550 nM to about 4 μM, e.g., between about 600 nM to about 4 μM, e.g., between about 650 nM to about 4 μM, e.g., between about 700 nM to about 4 μM, e.g., between about 750 nM to about 4 μM, e.g., between about 800 nM to about 4 μM, e.g., between about 1000 nM to about 4 μM, e.g., between about 1200 nM to about 4 μM, e.g., between about 1400 nM to about 4 μM, e.g., between about 1500 nM to about 4 μM; e.g., between about 200 nM (nanomolar) to about 3.5 μM (micromolar), e.g., between about 250 nM to about 3 μM, e.g., between about 300 nM to about 3 μM, e.g., between about 350 nM to about 3 μM, e.g., between about 400 nM to about 3 μM, e.g., between about 450 nM to about 3 μM, e.g., between about 550 nM to about 3 μM, e.g., between about 600 nM to about 3 μM, e.g., between about 650 nM to about 3 μM, e.g., between about 700 nM to about 3 μM, e.g., between about 750 nM to about 3 μM, e.g., between about 800 nM to about 3 μM, e.g., between about 1000 nM to about 3 μM, e.g., between about 1200 nM to about 3 μM, e.g., between about 1400 nM to about 3 μM, e.g., between about 1500 nM to about 3 μM; e.g., between about 200 nM (nanomolar) to about 2.5 μM (micromolar), e.g., between about 250 nM to about 2 μM, e.g., between about 300 nM to about 2 μM, e.g., between about 350 nM to about 2 μM, e.g., between about 400 nM to about 2 μM, e.g., between about 450 nM to about 2 μM, e.g., between about 550 nM to about 2 μM, e.g., between about 600 nM to about 2 μM, e.g., between about 650 nM to about 2 μM, e.g., between about 700 nM to about 2 μM, e.g., between about 750 nM to about 2 μM, e.g., between about 800 nM to about 2 μM, e.g., between about 1000 nM to about 2 μM, e.g., between about 1200 nM to about 2 μM, e.g., between about 1400 nM to about 2 μM, e.g., between about 1500 nM to about 2 μM; e.g., between about 200 nM (nanomolar) to about 2.0 μM (micromolar), e.g., between about 250 nM to about 1.5 μM, e.g., between about 300 nM to about 1.5 μM, e.g., between about 350 nM to about 1.5 μM, e.g., between about 400 nM to about 1.5 μM, e.g., between about 450 nM to about 1.5 μM, e.g., between about 550 nM to about 1.5 μM, e.g., between about 600 nM to about 1.5 μM, e.g., between about 650 nM to about 1.5 μM, e.g., between about 700 nM to about 1.5 μM, e.g., between about 750 nM to about 1.5 μM, e.g., between about 800 nM to about 1.5 μM, e.g., between about 1000 nM to about 1.5 μM, e.g., between about 1200 nM to about 1.5 μM, e.g., between about 1400 nM to about 1.5 μM.

According to some embodiments of the invention, the avidity of a cell (e.g., T cell or NK cells) transduced with the CAR molecule is determined using methods known in the art such as competition assays, titration assays and the like.

TCR intrinsic affinity is defined as the strength of binding of one TCR molecule to the antigen (e.g., peptide-MHC complex). In contrast, TCR binding avidity defines, in the cellular context, the strength of binding among multiple TCRs to their respective antigen (e.g., peptide-MHC complex) [reviewed in von Essen M R, Kongsbak M, Geisler C. Mechanisms behind functional avidity maturation in T cells. Clin Dev Immunol. 2012; 2012:163453; Stone J D, Chervin A S, Kranz D M. T-cell receptor binding affinities and kinetics: impact on T-cell activity and specificity. Immunology. 2009; 126(2):165-76; Harari, V. Dutoit, C. Cellerai, P. A. Bart, R. A. Du Pasquier, and G. Pantaleo, Functional signatures of protective antiviral T-cell immunity in human virus infections, Immunological Reviews, 2006.211: 236-254; each of which is fully incorporated herein in its entirety].

According to some embodiments of the invention, the avidity of the cell transduced with the CAR is determined by a peptide titration assay (e.g., detecting the peptide concentration at which there is 50% of Max interferon gamma release from the cell as described in the Examples section which follows, e.g., shown in FIG. 6B)

According to some embodiments of the invention, the avidity of the cell transduced with the CAR is between about 1 nM about 50 μM as determined by peptide titration assay (e.g., detecting the peptide concentration at which there is 50% of Max interferon gamma release from the cell as described in the Examples section which follows).

According to some embodiments of the invention, the avidity of the cell transduced with CAR is between about 1 nM to about 100 nM, e.g., between about 1-50 nM, e.g., between about 50-100 nM.

According to some embodiments of the invention, the avidity of the cell transduced with CAR is between about 100 nM to about 200 nM, e.g., between 50-200 nM.

According to some embodiments of the invention, the avidity of the cell transduced with CAR is between about 50-500 nM, e.g., between about 200-500 nM, e.g., about 300-500 nM, e.g., about 200-300 nM.

According to some embodiments of the invention, the avidity of the cell transduced with CAR is between about 500 nM and about 50 μM, e.g., between about 1-10 μM, e.g., between about 3-10 μM, e.g., between about 5-10 μM, e.g., between 10-50 μM, e.g., between about 10-30 μM, e.g., about 40-50 μM, e.g., about 5 μM, e.g., about 10 μM.

According to some embodiments of the invention, the avidity of the cell transduced with CAR is about 1-10 μM; e.g., about 3-10 μM, e.g., about 5 μM, or about 10 μM.

For example, an avidity of 5 μM was tested for T cells transduced with the WT1 αβTCR CAR used by the present inventors (FIG. 6B; see results of “TCR” in the upper graph showing interferon gamma release). Similarly, an avidity of 10 μM was tested for T cells transduced with the WT1 TCRL F2 CAR (FIG. 6B; see results of “F2” in the upper graph showing interferon gamma release).

Any numerical value described herein in the context of the term “about” should be read also in the absence of this term.

According to some embodiments of the invention, the extracellular domain comprises complementarity determining region (CDR) which are capable of specifically binding the antigen. Such CDRs can be obtained from an antibody.

The term “antibody” as used in this invention includes intact molecules as well as functional fragments thereof, such as Fab, Fab′, F(ab′)2, Fv, linear antibodies, scFv antibodies, and multispecific antibodies formed from antibody fragments that are capable of binding to the antigen. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)2, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; (5) Single chain antibody (“SCA”), a genetically engineered molecule containing the variable region of the light chain and the variable region of the heavy chain, linked by a suitable polypeptide linker as a genetically fused single chain molecule; (6) CDR peptide is a peptide coding for a single complementarity-determining region (CDR); and (7) Single domain antibodies (also called nanobodies), a genetically engineered single monomeric variable antibody domain which selectively binds to a specific antigen. Nanobodies have a molecular weight of only 12-15 kDa, which is much smaller than a common antibody (150-160 kDa).

An “antibody heavy chain,” as used herein, refers to the larger of the two types of polypeptide chains present in all antibody molecules in their naturally occurring c

An “antibody light chain,” as used herein, refers to the smaller of the two types of polypeptide chains present in all antibody molecules in their naturally occurring conformations. .kappa. and .lamda. light chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

Once the CDRs of an antibody are identified, using conventional genetic engineering techniques, expressible polynucleotides encoding any of the forms or fragments of antibodies described herein can be synthesized and modified in one of many ways in order to produce a spectrum of related-products.

According to some embodiments of the invention, the CDRs are derived from αβ T cell receptor (TCR) which specifically binds to the antigen.

According to some embodiments of the invention, the CDRs are derived from an engineered affinity-enhanced αβ T cell receptor (TCR) which specifically binds to the antigen, with a binding affinity characterized by a KD which is at least 150 nM.

According to some embodiments of the invention, the CDRs are derived from an engineered αβ T cell receptor (TCR) with improved stability or any other biophysical property.

According to some embodiments of the invention, the CDRs are derived from a T cell receptor-like (TCRLs) antibody which specifically binds to the antigen. Examples of TCRLs and methods of generating same are described in WO03/068201, WO2008/120203, WO2012/007950, WO2009125395, WO2009/125394, each of which is fully incorporated herein by their entirety.

According to some embodiments of the invention, the antigen binding domain comprises a single chain Fv (scFv) molecule.

According to some embodiments of the invention, the antigen binding domain comprises complementarity determining region (CDR) which specifically bind to the HLA-A2-WT1_{Db126} complexes.

According to some embodiments of the invention, the antigen binding domain comprises the complementarity determining region (CDR) of the F2 VL (SEQ ID NO:16; encoded by SEQ ID NO:18) and F2 VH (SEQ ID NO: 17; encoded by SEQ ID NO: 19) TCRL antibody.

According to some embodiments of the invention, the antigen binding domain of the CAR molecule binds a major histocompatibility complex (MHC) restricted antigen.

As used herein the phrase “major histocompatibility complex (MHC)” refers to a complex of antigens encoded by a group of linked loci, which are collectively termed H-2 in the mouse and human leukocyte antigen (HLA) in humans. The two principal classes of the MHC antigens, class I and class II, each comprise a set of cell surface glycoproteins which play a role in determining tissue type and transplant compatibility. In transplantation reactions, cytotoxic T-cells (CTLs) respond mainly against foreign class I glycoproteins, while helper T-cells respond mainly against foreign class II glycoproteins.

Major histocompatibility complex (MHC) class I molecules are expressed on the surface of nearly all cells. These molecules function in presenting peptides which are mainly derived from endogenously synthesized proteins to CD8+ T cells via an interaction with the αβ T-cell receptor. The class I MHC molecule is a heterodimer composed of a 46-kDa heavy chain which is non-covalently associated with the 12-kDa light chain β-2 microglobulin. In humans, there are several MHC haplotypes, such as, for example, HLA-A2, HLA-A1, HLA-A3, HLA-A24, HLA-A28, HLA-A31, HLA-A33, HLA-A34, HLA-B7, HLA-B45 and HLA-Cw8, their sequences can be found at the kabbat data base, at htexttransferprotocol://immuno.bme.nwu.edu. Further information concerning MHC haplotypes can be found in Paul, B. Fundamental Immunology Lippincott-Rven Press.

As used herein the term “peptide” refers to native peptides (either proteolysis products or synthetically synthesized peptides) and further to peptidomimetics, such as peptoids and semipeptoids which are peptide analogs, which may have, for example, modifications rendering the peptides more stable while in a body, or more immunogenic. Such modifications include, but are not limited to, cyclization, N terminus modification, C terminus modification, peptide bond modification, including, but not limited to, CH₂—NH, CH₂—S, CH₂—S═O, O═C—NH, CH₂—O, CH₂—CH₂, S═C—NH, CH═CH or CF═CH, backbone modification and residue modification. Methods for preparing peptidomimetic compounds are well known in the art and are specified in Quantitative Drug Design, C. A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the peptide may be substituted, for example, by N-methylated bonds (—N(CH3)-CO—), ester bonds (—C(R)H—C—O—O—C(R)—N—), ketomethylen bonds (—CO—CH2-), α-aza bonds (—NH—N(R)—CO—), wherein R is any alkyl, e.g., methyl, carba bonds (—CH2-NH—), hydroxyethylene bonds (—CH(OH)—CH2-), thioamide bonds (—CS—NH—), olefinic double bonds (—CH═CH—), retro amide bonds (—NH—CO—), peptide derivatives (—N(R)—CH2-CO—), wherein R is the “normal” side chain, naturally presented on the carbon atom.

These modifications can occur at any of the bonds along the peptide chain and even at several (2-3) at the same time. According to some embodiments of the invention, but not in all cases necessary, these modifications should exclude anchor amino acids.

Natural aromatic amino acids, Trp, Tyr and Phe, may be substituted for synthetic non-natural acid such as TIC, naphthylelanine (Nol), ring-methylated derivatives of Phe, halogenated derivatives of Phe or o-methyl-Tyr.

In addition to the above, the peptides of the invention may also include one or more modified amino acids or one or more non-amino acid monomers (e.g. fatty acids, complex carbohydrates etc).

As used herein in the specification and in the claims section below the term “amino acid” is understood to include the 20 naturally occurring amino acids; those amino acids often modified post-translationally in vivo, including for example hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids. Further elaboration of the possible amino acids usable according to the present invention and examples of non-natural amino acids useful in MHC-I HLA-A2 recognizable peptide antigens are given herein under.

The peptides of the invention are preferably utilized in a linear form, although it will be appreciated that in cases where cyclicization does not severely interfere with peptide characteristics, cyclic forms of the peptide can also be utilized.

The peptides of the invention may include one or more non-natural or natural polar amino acids, including but not limited to serine and threonine which are capable of increasing peptide solubility due to their hydroxyl-containing side chain.

The peptides of the invention may be synthesized by any techniques that are known to those skilled in the art of peptide synthesis. For solid phase peptide synthesis, a summary of the many techniques may be found in J. M. Stewart and J. D. Young, Solid Phase Peptide Synthesis, W. H. Freeman Co. (San Francisco), 1963 and J. Meienhofer, Hormonal Proteins and Peptides, vol. 2, p. 46, Academic Press (New York), 1973. For classical solution synthesis see G. Schroder and K. Lupke, The Peptides, vol. 1, Academic Press (New York), 1965. Large scale peptide synthesis is described by Andersson Biopolymers 2000; 55(3):227-50.

Based on accumulated experimental data, it is nowadays possible to predict which of the peptides of a protein will bind to MHC, class I. The HLA-A2 MHC class I has been so far characterized better than other HLA haplotypes, yet predictive and/or sporadic data is available for all other haplotypes.

With respect to HLA-A2 binding peptides, assume the following positions (P1-P9) in a 9-mer peptide:

P1-P2-P3-P4-P5-P6-P7-P8-P9

The P2 and P2 positions include the anchor residues which are the main residues participating in binding to MHC molecules. Amino acid resides engaging positions P2 and P9 are hydrophilic aliphatic non-charged natural amino (examples being Ala, Val, Leu, Ile, Gln, Thr, Ser, Cys, preferably Val and Leu) or of a non-natural hydrophilic aliphatic non-charged amino acid (examples being norleucine (Nle), norvaline (Nva), α-aminobutyric acid). Positions P1 and P3 are also known to include amino acid residues which participate or assist in binding to MHC molecules, however, these positions can include any amino acids, natural or non-natural. The other positions are engaged by amino acid residues which typically do not participate in binding, rather these amino acids are presented to the immune cells. Further details relating to the binding of peptides to MHC molecules can be found in Parker, K. C., Bednarek, M. A., Coligan, J. E., Scheme for ranking potential HLA-A2 binding peptides based on independent binding of individual peptide side-chains. J Immunol. 152,163-175, 1994., see Table V, in particular. Hence, scoring of HLA-A2.1 binding peptides can be performed using the HLA Peptide Binding Predictions software approachable through a worldwide web interface at hypertexttransferprotocol://worldwideweb.bimas.dcrt.nih.gov/molbio/hla_bind/index. This software is based on accumulated data and scores every possible peptide in an analyzed protein for possible binding to MHC HLA-A2.1 according to the contribution of every amino acid in the peptide. Theoretical binding scores represent calculated half-life of the HLA-A2.1-peptide complex.

Hydrophilic aliphatic natural amino acids at P2 and P9 can be substituted by synthetic amino acids, preferably Nleu, Nval and/or α-aminobutyric acid. P9 can be also substituted by aliphatic amino acids of the general formula —HN(CH₂)ₙCOOH, wherein n=3-5, as well as by branched derivatives thereof, such as, but not limited to,

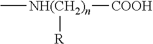

wherein R is, for example, methyl, ethyl or propyl, located at any one or more of the n carbons.

The amino terminal residue (position P1) can be substituted by positively charged aliphatic carboxylic acids, such as, but not limited to, H₂N(CH₂)ₙCOOH, wherein n=2-4 and H₂N—C(NH)—NH(CH₂)ₙCOOH, wherein n=2-3, as well as by hydroxy Lysine, N-methyl Lysine or ornithine (Orn). Additionally, the amino terminal residue can be substituted by enlarged aromatic residues, such as, but not limited to, H₂N—(C₆H₆)—CH₂—COOH, p-aminophenyl alanine, H₂N—F(NH)—NH—(C₆H₆)—CH₂—COOH, p-guanidinophenyl alanine or pyridinoalanine (Pal). These latter residues may form hydrogen bonding with the OH⁻ moieties of the Tyrosine residues at the MHC-1 N-terminal binding pocket, as well as to create, at the same time aromatic-aromatic interactions.

Derivatization of amino acid residues at positions P4-P8, should these residues have a side-chain, such as, OH, SH or NH₂, like Ser, Tyr, Lys, Cys or Orn, can be by alkyl, aryl, alkanoyl or aroyl. In addition, OH groups at these positions may also be derivatized by phosphorylation and/or glycosylation. These derivatizations have been shown in some cases to enhance the binding to the T cell receptor.

Longer derivatives in which the second anchor amino acid is at position P10 may include at P9 most L amino acids. In some cases shorter derivatives are also applicable, in which the C terminal acid serves as the second anchor residue.

Cyclic amino acid derivatives can engage position P4-P8, preferably positions P6 and P7. Cyclization can be obtained through amide bond formation, e.g., by incorporating Glu, Asp, Lys, Orn, di-amino butyric (Dab) acid, di-aminopropionic (Dap) acid at various positions in the chain (—CO—NH or —NH—CO bonds). Backbone to backbone cyclization can also be obtained through incorporation of modified amino acids of the formulas H—N((CH₂)ₙ—COOH)—C(R)H—COOH or H—N((CH₂)ₙ—COOH)—C(R)H—NH₂, wherein n=1-4, and further wherein R is any natural or non-natural side chain of an amino acid.

Cyclization via formation of S—S bonds through incorporation of two Cys residues is also possible. Additional side-chain to side chain cyclization can be obtained via formation of an interaction bond of the formula —(—CH₂—)ₙ—S—CH₂—C—, wherein n=1 or 2, which is possible, for example, through incorporation of Cys or homoCys and reaction of its free SH group with, e.g., bromoacetylated Lys, Orn, Dab or Dap.

Peptide bonds (—CO—NH—) within the peptide may be substituted by N-methylated bonds (—N(CH₃)—CO—), ester bonds (—C(R)H—C—O—O—C(R)—N—), ketomethylen bonds (—CO—CH₂—), α-aza bonds (—NH—N(R)—CO—), wherein R is any alkyl, e.g., methyl, carba bonds (—CH₂—NH—), hydroxyethylene bonds (—CH(OH)—CH₂—), thioamide bonds (—CS—NH—), olefinic double bonds (—CH═CH—), retro amide bonds (—NH—CO—), peptide derivatives (—N(R)—CH₂—CO—), wherein R is the “normal” side chain, naturally presented on the carbon atom.

These modifications can occur at any of the bonds along the peptide chain and even at several (2-3) at the same time. Preferably, but not in all cases necessary, these modifications should exclude anchor amino acids.

Natural aromatic amino acids, Trp, Tyr and Phe, may be substituted for synthetic non-natural acid such as TIC, naphthylelanine (Nol), ring-methylated derivatives of Phe, halogenated derivatives of Phe or o-methyl-Tyr.

As used herein the phrase “tumor antigen” refers to an antigen that is common to specific hyperproliferative disorders such as cancer. Tumor antigens are proteins that are produced by tumor cells that elicit an immune response, particularly T-cell mediated immune responses. The selection of the antigen binding moiety of the invention will depend on the particular type of cancer to be treated.

The type of tumor antigen referred to in the invention includes a tumor-specific antigen (TSA) or a tumor-associated antigen (TAA). A “TSA” is unique to tumor cells and does not occur on other cells in the body. A “TAA” is not unique to a tumor cell and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development when the immune system is immature and unable to respond or they may be antigens that are normally present at extremely low levels on normal cells but which are expressed at much higher levels on tumor cells.

The antigens discussed herein are merely included by way of example. The list is not intended to be exclusive and further examples will be readily apparent to those of skill in the art.

Tumor antigens are well known in the art and include, for example, a glioma-associated antigen, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin. These molecules include but are not limited to tissue-specific antigens such as MART-1, tyrosinase and GP 100 in melanoma and prostatic acid phosphatase (PAP) and prostate-specific antigen (PSA) in prostate cancer. Other target molecules belong to the group of transformation-related molecules such as the oncogene HER-2/Neu/ErbB-2. Yet another group of target antigens are onco-fetal antigens such as carcinoembryonic antigen (CEA). In B-cell lymphoma the tumor-specific idiotype immunoglobulin constitutes a truly tumor-specific immunoglobulin antigen that is unique to the individual tumor. B-cell differentiation antigens such as CD19, CD20 and CD37 are other candidates for target antigens in B-cell lymphoma. Some of these antigens (CEA, HER-2, CD19, CD20, idiotype) have been used as targets for passive immunotherapy with monoclonal antibodies with limited success.

Non-limiting examples of TSA or TAA antigens include the following: Differentiation antigens such as MART-1/MelanA (MART-1), gp100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.291\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, and TPS.

In a preferred embodiment, the antigen binding moiety portion of the CAR targets an antigen that includes but is not limited to CD19, CD20, CD22, ROR1, Mesothelin, CD33/IL3Ra, c-Met, PSMA, Glycolipid F77, EGFRvIII, GD-2, MY-ESO-1 TCR, MAGE A3 TCR, and the like.

Following are non-limiting sequences of HLA class I-restricted tumor antigens which can bind to the antigen binding domain of the CAR molecule of the invention.

TABLE 1

| | | | SEQ ID | |
|---|---|---|---|---|
| | | | NO: of | |
| | | | the | |
| | | GenBank Accession No. of the tumor | tumor | |
| Cancer | TAA/Marker | antigens | antigens | HLA |
| Transitional | Uroplakin II | NP_006751.1 | 94 | HLA- |
| cell | (UPKII) | | | A2 |
| carcinoma | | | | |
| Transitional | Uroplakin Ia | NP_001268372.1; NP_008931.1 | 95, 96 | HLA- |
| cell | (UPK1A) | | | A2 |
| carcinoma | | | | |
| Carcinoma of | prostate | AAO16090.1 | 97 | HLA- |
| the | specific | | | A2 |
| prostate | antigen | | | |
| | (NPSA) | | | |
| Carcinoma of | prostate | NP_005663.2 | 98 | HLA- |
| the | specific | | | A2 |
| prostate | membrane | | | |
| | antigen | | | |
| | (PSCA) | | | |
| Carcinoma of | prostate acid | NP_001090.2; NP_001127666.1; | 99-101 | HLA- |
| the | phosphatase | NP_001278966.1 | | A2 |
| prostate | (ACPP) | | | |
| Breast cancer | BA-46 | NP_001108086.1; NP_005919.2; | 102-103 | HLA- |
| | MFGE8 milk | | | A2 |
| | fat globule- | | | |
| | EGF factor 8 | | | |
| | protein | | | |
| | [lactadherin] | | | |
| Breast cancer | Mucin 1 | NP_001018016.1; NP_001018017.1; | 104-123 | HLA- |
| | (MUC1) | NP_001037855.1; NP_001037856.1; | | A2 |
| | | NP_001037857.1; NP_001037858.1; | | |
| | | NP_001191214.1; NP_001191215.1; | | |
| | | NP_001191216.1; NP_001191217.1; | | |
| | | NP_001191218.1; NP_001191219.1; | | |
| | | NP_001191220.1; NP_001191221.1; | | |
| | | NP_001191222.1; NP_001191223.1; | | |
| | | NP_001191224.1; NP_001191225.1; | | |
| | | NP_001191226.1; NP_002447.4 | | |
| Melanoma | premelanosome | NP_001186982.1; NP_001186983.1; | 124-126 | HLA- |
| | protein | NP_008859.1 | | A2 |
| | (PMEL; also | | | |
| | known as | | | |
| | Gp100) | | | |
| Melanoma | melan-A | NP_005502.1; | 127 | HLA- |
| | (MLANA; | | | A2 |
| | also known as | | | |
| | MART1) | | | |
| All tumors | telomerase | NP_001180305.1; NP_937983.2 | 128-129 | HLA- |
| | reverse | | | A2 |
| | transcriptase | | | |
| | (TERT) | | | |
| Leukemia and | TAX | NP_057864.1; YP_002455788.1 | 130-131 | HLA- |
| Burkitts | tax p40 | | | A2 |
| Lymphoma | [Human T- | | | |
| | lymphotropic | | | |
| | virus 1] and | | | |
| | Tax [Human | | | |
| | T- | | | |
| | lymphotropic | | | |
| | virus 4]; | | | |
| Carcinomas | NY-ESO | NP_001318.1 | 132 | HLA- |
| | cancer/testis | | | A2 |
| | antigen 1B | | | |
| | (CTAG1B) | | | |
| Melanoma | Melanoma | NP_004979.3 | 133 | HLA- |
| | antigen family | | | A2 |
| | A1 | | | |
| | (MAGEA1) | | | |
| Melanoma | Melanoma | NP_005353.1 | 134 | HLA- |
| | antigen family | | | A24 |
| | A3 | | | |
| | (MAGEA3, | | | |
| | MAGE-A3) | | | |
| Carcinomas | HER2; erb-b2 | NP_001005862.1; NP_001276865.1; | 135-139 | HLA- |
| | receptor | NP_001276866.1; NP_001276867.1; | | A2 |
| | tyrosine kinase | NP_004439.2; | | |
| | 2 (ERBB2) | | | |
| Melanoma | Beta-catenine; | NP_001091679.1; NP_001091680.1; | 140-142 | HLA- |
| | catenin | NP_001895.1; | | A24 |
| | (cadherin- | | | |
| | associated | | | |
| | protein), beta | | | |
| | 1, 88 kDa | | | |
| | (CTNNB1) | | | |
| Melanoma | Tyrosinase | NP_000363.1 | 143 | HLA- |
| | (TYR) | | | DRB1 |
| Leukemia | Bcr-abl | AAA35594.1 | 144 | HLA- |
| | | | | A2 |
| Head and | caspase 8, | NP_001073593.1; NP_001073594.1; | 145-150 | HLA- |
| neck | apoptosis- | NP_001219.2; NP_203519.1; | | B35 |
| | related | NP_203520.1; NP_203522.1 | | |
| | cysteine | | | |
| | peptidase | | | |
| | (CASP8) | | | |

Table 1.

According to some embodiments of the invention, the tumor associated antigen comprises the WT1 protein.

According to some embodiments of the invention, the MHC-restricted tumor associated antigen is the WT1_{Db126} peptide set forth in SEQ ID NO:1.

According to some embodiments of the invention, the tumor associated antigen comprises the tyrosinase protein.

Tyrosinase peptides that bind to class I MHC molecules (also referred to herein interchangeably as HLA-restricted tyrosinase epitopes, HLA-restricted tyrosinase epitopes and MHC-restricted tyrosinase antigens) are derived from the tyrosinase enzyme (Genebank Accession No: NP_000363.1, SEQ ID NO:143) and are typically 8-10 amino acids long, bind to the heavy chain α1-α2 groove via two or three anchor residues that interact with corresponding binding pockets in the MHC molecule.

Tyrosinase is a membrane-associated N-linked glycoprotein and it is the key enzyme in melanin synthesis. It is expressed in all healthy melanocytes and in nearly all melanoma tumor samples (H. Takeuchi, et al., 2003; S. Reinke, et al., 2005). Peptides derived from this enzyme are presented on MHC class I molecules and are recognized by autologuos cytolytic T lymphocytes in melanoma patients [T. Wolfel, et al., 1994; Brichard, et al., 1993; Renkvist et al, Cancer immunology immunotherapy 2001 50:3-15; Novellino L, et al., March 2004 update. Cancer Immunol Immunother. 54:187-207, 2005]. Additional tumor tyrosinase HLA-restricted peptides derived from tumor associated antigens (TAA) can be found at the website of the Istituto Nazionale per lo Studio e la Cura dei Tumori at hypertexttransferprotocol://worldwideweb.istitutotumori.mi.it.

Non-limiting examples of MHC class I restricted tyrosinase antigenic peptides are provided in WO2008/120202, which is fully incorporated herein by reference in its entirety, e.g., in Table 139 of WO2008/120202.

According to some embodiments of the invention, the tyrosinase antigenic peptide is the Tyrosinase₃₆₉₋₃₇₇ peptide [YMDGTMSQV; SEQ ID NO:8].

According to some embodiments of the invention, the MART-1 antigenic peptide is the peptide set forth by SEQ ID NO:9 (EAAGIGILTV).

Depending on the desired antigen to be targeted, the CAR molecule of the invention can be engineered to include the appropriate antigen bind moiety that is specific to the desired antigen target.

For example, if WT1 is the desired antigen that is to be targeted, an antibody for a complex between MHC and WT1_{Db126} peptide (SEQ ID NO:1) can be used as the antigen bind moiety for incorporation into the CAR of the invention.

For example, if tyrosinase is the desired antigen that is to be targeted, an antibody for a complex between MHC and Tyrosinase₃₆₉₋₃₇₇ peptide [YMDGTMSQV; SEQ ID NO:8] can be used as the antigen bind moiety for incorporation into the CAR of the invention.

Following are non-limiting sequences of HLA class I-restricted viral antigens which can bind to the antigen binding domain of the CAR molecule of the invention (Table 2 below).

According to some embodiments of the invention, the viral antigens include viral epitopes from a polypeptide selected from the group consisting of: human T cell lymphotropic virus type I (HTLV-1) transcription factor (TAX), influenza matrix protein epitope, Epstein-Bar virus (EBV)-derived epitope, HIV-1 RT, HIV Gag, HIV Pol, influenza membrane protein M1, influenza hemagglutinin, influenza neuraminidase, influenza nucleoprotein, influenza nucleoprotein, influenza matrix protein (M1), influenza ion channel (M2), influenza non-structural protein NS-1, influenza non-structural protein NS-2, influenza PA, influenza PB1, influenza PB2, influenza BM2 protein, influenza NB protein, influenza nucleocapsid protein, Cytomegalovirus (CMV) phosphorylated matrix protein (pp65), TAX, hepatitis C virus (HCV), HBV pre-S protein 85-66, HTLV-1 tax 11-19, HBV surface antigen 185-194.

TABLE 2

| | | GenBank | | |
|---|---|---|---|---|
| | | Accession Nos. | | |
| | | of the viral | SEQ ID NOs: of | |
| | | antigens; or the | the viral | |
| Disease | Viral antigen | peptide sequence | antigens | HLA |
| AIDS (HTLV-1) | HIV-1 RT 476-484 | | | HLA-A2 |
| Gag (HIV) | Gag 77-85 | SLYNTVATL | 230 | |
| Pol (HIV) | Pol 476-484 | ILEPVHGV | 231 | |
| Influenza | | GILGFVFTL | 232 | HLA-A2 |
| Influenza | Membrane | YP_308854.1 | 233 | |
| | protein M1 of | | | |
| | influenza A virus | | | |
| | A/Korea/426/68 | | | |
| | (H2N2) | | | |
| Influenza | hemagglutinin of | NP_056660.1; | 234-235 | |
| | influenza B | YP_308839.1 | | |
| | virus; | | | |
| | hemagglutinin of | | | |
| | influenza A virus | | | |
| | (A/New | | | |
| | York/392/2004 | | | |
| | (H3N2) | | | |
| Influenza | neuraminidase of | NP_056663.1 | 236 | |
| | influenza B virus | | | |
| Influenza | nucleoprotein of | YP_089656.1 | 237 | |
| | influenza C | | | |
| | virus; | | | |
| Influenza | nucleoprotein of | YP_308871.1; | 238-239 | |
| | influenza A virus | YP_581749.1; | | |
| | such as the | | | |
| | A/Korea/426/68 | | | |
| | (H2N2) strain; or | | | |
| | the A/Hong | | | |
| | Kong/1073/99 (H9N2) | | | |
| | strain | | | |
| Influenza | nucleoprotein of | NP_056661.1; | 240 | |
| | influenza B virus | | | |
| Influenza | matrix protein | NP_056664.1 | 241 | |
| | (M1) of | | | |
| | influenza B virus | | | |
| Influenza | ion channel (M2) | NP_040979.2 | 242 | |
| | of influenza A | | | |
| | virus A/Puerto | | | |
| | Rico/8/34 (H1N1) | | | |
| | strain | | | |
| Influenza | non-structural | NP_056666.1 | 243 | |
| | protein NS-1 of | | | |
| | influenza B virus | | | |
| Influenza | non-structural | NP_056665.1 | 244 | |
| | protein NS-2 of | | | |
| | influenza B virus | | | |
| Influenza | PA of influenza | AAL60433 | 245 | |
| | A virus | | | |
| | A/Charlottesville/ | | | |
| | 28/95 (H1N1) | | | |
| Influenza | PB1 of | NP_056657.1 | 246 | |
| | influenza B virus | | | |
| Influenza | PB2 of influenza | NP_040987.1 | 247 | |
| | A virus | | | |
| | (A/Puerto | | | |
| | Rico/8/34 (H1N1) | | | |
| Influenza | BM2 protein of | YP_419283.1 | 248 | |
| | influenza B virus | | | |
| Influenza | NB protein of | NP_056662.1 | 249 | |
| | influenza B virus | | | |
| Influenza | nucleocapsid | NP_040982.1 | 250 | |
| | protein of | | | |
| | influenza A virus | | | |
| | A/Puerto | | | |
| | Rico/8/34 (H1N1) | | | |
| CMV disease | CMV | AAA45996; | 10; 11; 251; 252 | HLA-A2 |
| | phosphorylated | P06725; | | |
| | matrix protein | AAA45994.1; | | |
| | (pp65) [Human | P18139 | | |
| | herpesvirus 5] | | | |
| Leukemia and | TAX | NP_057864.1; | 130-131 | HLA-A2 |
| Burkitts | tax p40 [Human | YP_002455788.1 | | |
| Lymphoma | T-lymphotropic | | | |
| | virus 1] and | | | |
| | Tax [Human T- | | | |
| | lymphotropic | | | |
| | virus 4]; | | | |
| Hepatitis C | HCV | | | HLA-A2 |
| Hepatitis B | HBV pre-S | STNRQSGRQ | 253 | HLA-A2 |
| | protein | | | |
| | 85-66 | | | |
| HTLV-1 | HTLV-1 tax 11-19 | LLFGYPVYV | 254 | HLA-A2 |
| Leukemia | | | | |
| Hepatitis | HBV surface | GLSPTVWLSV | 255 | HLA-A2 |
| | antigen 185-194 | | | |

Cytomegalovirus (CMV) belongs to the human herpesviruses. There are several known strains of CMV, including strains 1042, 119, 2387, 4654, 5035, 5040, 5160, 5508, AD169, Eisenhardt, Merlin, PT, Toledo and Towne. During viral infection, the expressed viral proteins, e.g., pp65 of the CMV AD169 strain [GenBank Accession No. AAA45996.1 (SEQ ID NO:10); or GenBank Accession No. P06725 (SEQ ID NO:11)] pp64 of the CMV Towne strain [GenBank Accession No. AAA45994.1 for amino acids (SEQ ID NO:251); or GenBank Accession No. P18139 (SEQ ID NO:252)] are subject to proteasomal degradation and the MHC-restricted peptides bind to the MHC molecules [e.g., MHC class I or MHC class II] and are further presented therewith on the cell surface. The pp65 (561 amino acids in length) and pp64 (551 amino acids in length) proteins of the CMV AD169 and Towne strains, respectively, are 99% identical proteins and share the same amino acid sequence from position 3-551 of pp64 and 13-561 of pp65.

According to some embodiments of the invention, the MHC-restricted CMV antigenic peptide is the antigenic peptide derived from the pp65 or pp64 proteins and described in Table 137 of WO2008/120203, which is fully incorporated herein by reference in its entirety.

According to some embodiments of the invention, the MHC restricted antigen comprises an MHC class II restricted antigen.

MHC class II molecules are expressed in professional antigen presenting cells (APCs) such as macrophages, dendritic cells and B cells. Each MHC class II molecule is a heterodimer composed of two homologous subunits, alpha chain (with α1 and α2 extracellular domains, transmembrane domain and short cytoplasmic tail) and beta chain (with β1 and β2 extracellular domains, transmembrane domain and short cytoplasmic tail). Peptides, which are derived from extracellular proteins, enter the cells via endocytosis, are digested in the lysosomes and further bind to MHC class II molecules for presentation on the membrane.

Various MHC class II molecules are found in humans. Examples include, but are not limited to HLA-DM, HLA-DO, HLA-DP, HLA-DQ (e.g., DQ2, DQ4, DQ5, DQ6, DQ7, DQ8, DQ9), HLA-DR (e.g., DR1, DR2, DR3, DR4, DR5, DR7, DR8, DR9, DR10, DR11, DR12, DR13, DR14, DR15, and DR16).

Non-limiting examples of DQ A1 alleles include 0501, 0201, 0302, 0301, 0401, 0101, 0102, 0104, 0102, 0103, 0104, 0103, 0102, 0303, 0505 and 0601.

Non-limiting examples of DQ B1 alleles include 0201, 0202, 0402, 0501, 0502, 0503, 0504, 0601, 0602, 0603, 0604, 0609, 0301, 0304, 0302 and 0303.

Non-limiting examples of DPA1 alleles include 01, e.g., 0103, 0104, 0105, 0106, 0107, 0108, 0109; 02, e.g., 0201, 0202, 0203; 03 e.g., 0301, 0302, 0303, 0401.

Non-limiting examples of DPB1 alleles include 01, e.g., 0101, 0102; 02 e.g., 0201, 0202, 0203; 03; 04, e.g., 0401, 0402, 0403; 05, e.g., 0501, 0502; 06; 08, e.g., 0801, 0802; 09, e.g., 0901, 0902; 10, e.g., 1001, 1002; 11 e.g., 1101, 1102; 13, e.g., 1301, 1302; 14, e.g., 1401, 1402; 15, e.g., 1501, 1502; 16, e.g., 1601, 1602; 17, e.g., 1701, 1702; 18, e.g., 1801, 1802; 19, e.g., 1901, 1902; 20, e.g., 2001, 2002; 21; 22; 23; 24; 25; 26, e.g., 2601, 2602; and 27.

Non-limiting examples of DP haplotypes include HLA-DPA1*0103/DPB1*0401 (DP401); and HLA-DPA1*0103/DPB1*0402 (DP402).

Non-limiting examples of DR B1 alleles include 0101, 0102, 0103, 0301, 0401, 0407, 0402, 0403, 0404, 0405, 0701, 0701, 0801, 0803, 0901, 1001, 1101, 1103, 1104, 1201, 1301, 1302, 1302, 1303, 1401, 1501, 1502, 1601 alleles.

Non-limiting examples of DR-DQ haplotypes include DR1-DQ5, DR3-DQ2, DR4-DQ7, DR4-DQ8, DR7-DQ2, DR7-DQ9, DR8-DQ4, DR8-DQ7, DR9-DQ9, DR10-DQ5, DR11-DQ7, DR12-DQ7, DR13-DQ6, DR13-DQ7, DR14-DQ5, DR15-DQ6, and DR16-DQ5.

According to some embodiments of the invention, the antigen is an autoantigen associated with an autoimmune disease.

The term “autoimmune disease” as used herein is defined as a disorder that results from an autoimmune response. An autoimmune disease is the result of an inappropriately excessive response to a self-antigen.

Examples of autoimmune diseases include but are not limited to, Addision's disease, alopecia greata, ankylosing spondylitis, autoimmune hepatitis, autoimmune parotitis, Crohn's disease, diabetes (Type 1), dystrophic epidermolysis bullosa, epididymitis, glomerulonephritis, Graves' disease, Guillain-Barr syndrome, Hashimoto's disease, hemolytic anemia, systemic lupus erythematosus, multiple sclerosis, myasthenia gravis, pemphigus vulgaris, psoriasis, rheumatic fever, rheumatoid arthritis, sarcoidosis, scleroderma, Sjogren's syndrome, spondyloarthropathies, thyroiditis, vasculitis, vitiligo, myxedema, pernicious anemia, ulcerative colitis, among others.

As used herein the phrase “autoantigenic peptide” refers to an antigen derived from an endogenous (i.e., self protein) or a consumed protein (e.g., by food) against which an inflammatory response is elicited as part of an autoimmune inflammatory response.

It should be noted that the phrases “endogenous”, “self” are relative expressions referring to the individual in which the autoimmune response is elicited. Auto-antigens comprise, but are not limited to, cellular proteins, phosphoproteins, cellular surface proteins, cellular lipids, nucleic acids, glycoproteins, including cell surface receptors.

It should be noted that presentation of an autoantigenic peptide on antigen presenting cells (APCs) can result in recognition of the MHC-autoantigenic peptides by specific T cells, and consequently generation of an inflammatory response that can activate and recruit T cell and B cell responses against the APCs cells.

A common basis for several autoimmune diseases, including Multiple Sclerosis (MS), Type 1 Diabetes (T1D) and Rheumatoid Arthritis (RA), is the strong linkage between HLA genotype and susceptibility to the disease (Nepom, 1991; Sawcer, 2005; McDaniel, 1989). While some alleles are tightly linked to certain diseases, others confer protection and are extremely rare in patients. This linkage is not surprising due to the involvement of T-cells in the progression of these diseases. Activation or disregulation of CD4+ T-cells directed to self organ-specific proteins, combined with yet-undefined events, may contribute to the pathogenesis of a variety of human autoimmune diseases.

Multiple sclerosis is an immune-mediated demyelinating and neurodegenerative disease of the central nervous system (CNS) (Trapp, 2008). Susceptibility to MS is associated with human leukocyte antigen (HLA) class II alleles, mostly the DR2 haplotype that includes the DRB1*1501, DRB1*0101, and DQB1*0602 genes (Olerup, 1991). DRB1*1501 is a well-studied risk factor of MS that occurs in about 60% of Caucasian MS patients vs. 25% of healthy controls. Contribution of these risk factors to disease process likely involves presentation of self antigens by disease-associated MHC expressed on antigen presenting cells (APC) that activate T-cell-mediated central nervous system (CNS) inflammation. Suspected MS autoantigens include myelin proteins such as myelin basic protein (MBP), proteolipid protein (PLP), and myelin oligodendrocyte glycoprotein (MOG). T-cells from MS patients were found to predominantly recognize MOG (Kerlero de rosbo, 1993; Kerlero de rosbo, 1998) as well as other myelin proteins, and the MOG-35-55 peptide was found to be highly encephalitogenic in rodents and monkeys (Mendel, 1995; Johns, 1995) and induces severe chronic experimental autoimmune encephalomyelitis (EAE) in HLA-DRB1*1501-Tg mice (Rich, 2004).

Type 1 Diabetes (T1D) involves progressive destruction of pancreatic beta-cells by autoreactive T-cells specific for antigens expressed in the pancreatic islets, including glutamic acid decarboxylase (GAD65) (Karslen, 1991). GAD65 is a suspected islet autoantigen in T1D, stimulating both humoral and cellular self reactivity in at-risk and diseased subjects. Antibodies to GAD65 in combination with antibodies directed at two additional islet autoantigens are predictive markers of T1D in at-risk subjects (Verge, 1996), and GAD-555-567 peptide has identical sequence in all GAD isoforms in human and mouse. This highly immunogenic determinant was found to be a naturally processed T-cell epitope both in disease-associated-HLA-DR4(*0401)-Tg-mice (Patel, 1997) and human T1D subjects (Reijonen, 2002; Nepom, 2001).

Celiac (Coeliac) is an autoimmune disorder of the small intestine that occurs in genetically predisposed people of all ages from middle infancy onward. Celiac is caused by a reaction to gliadin, a prolamin (gluten protein) found in wheat, and similar proteins found in the crops of the tribe Triticeae (e.g., barley and rye). Upon exposure to gliadin, and specifically to two peptides found in prolamins (Gliadin-61-71 and Gliadin-3-24) the immune system cross-reacts with the small-bowel tissue, causing an inflammatory reaction.

According to some embodiments of the invention the autoantigenic peptide is associated with a disease selected from the group consisting of diabetes, multiple sclerosis, rheumatoid arthritis, celiac disease and stroke.

As used herein the phrase “type I diabetes-associated autoantigenic peptide” refers to an antigen derived from a self protein (i.e., an endogenous protein), which is expressed in pancreatic cells such as beta cells of the pancreas, and against which an inflammatory response is elicited as part of an autoimmune inflammatory response. According to some embodiments of the invention, the diabetes-associated autoantigenic peptide is a beta-cell autoantigenic peptide.

It should be noted that a type I diabetes-associated autoantigenic peptide is an MHC class II-restricted peptide, which when presented on antigen presenting cells (APCs) is recognized by specific T cells. Such a presentation by APCs generates an inflammatory response that can activate and recruit T cell and B cell responses against beta cells, including the generation of cytotoxic T cells and antibodies which kill and destroy beta cells and thus lead to a decreased insulin production.

According to some embodiments of the invention, the diabetes-associated autoantigenic peptide is derived from a polypeptide selected from the group consisting of preproinsulin (amino acids 1-110 of GenBank Accession No. NP_000198, SEQ ID NO:36), proinsulin (amino acids 25-110 of GenBank Accession No. NP_000198, SEQ ID NO: 37), Glutamic acid decarboxylase (GAD, GenBank Accession No. NP_000809.1, SEQ ID NO: 38), Insulinoma Associated protein 2 (IA-2, GenBank accession No. NP_115983) SEQ ID NO: 39), IA-2β [also referred to as phogrin, GenBank Accession No. NP_570857.2 (SEQ ID NO: 40), NP_570858.2 (SEQ ID NO:41), NP_002838.2 (SEQ ID NO: 42)], Islet-specific Glucose-6-phosphatase catalytic subunit-Related Protein [IGRP; GeneID: 57818, GenBank Accession No. NP_066999.1, glucose-6-phosphatase 2 isoform 1 (SEQ ID NO: 43) and GenBank Accession No. NP_001075155.1, glucose-6-phosphatase 2 isoform 2 (SEQ ID NO: 44)], chromogranin A (GenBank Accession No. NP_001266 (SEQ ID NO: 45), Zinc Transporter 8 (ZnT8; GenBank Accession NO. NP_776250.2, SEQ ID NO: 46), Heat Shock Protein-60 (GenBank Accession No. NP_955472.1; SEQ ID NO: 47), and Heat Shock Protein-70 (GenBank Accession No. NP_005337.2 (SEQ ID NO: 48) and NP_005336.3 (SEQ ID NO: 49).

As used herein the phrase “glutamic acid decarboxylase (GAD)” refers to a family of proteins which are responsible for catalyzing the production of gamma-aminobutyric acid from L-glutamic acid. There are two major GAD enzymes in humans, GAD 65 kDa which is expressed in both brain and pancreas (Gene ID 2572; encoded by GenBank accession No. NM_000818.2 (SEQ ID NO:50); NM_001134366.1 (SEQ ID NO:51); NP_000809.1 (SEQ ID NO:38)] and GAD 67 kDa which is expressed in brain [GeneID 2571; encoded by GenBank accession No. NM_000817.2 (SEQ ID NO:52); NP_000808.2 (SEQ ID NO:53)]. GAD 65 kDa has been identified as an autoantibody and an autoreactive T cell target in insulin-dependent diabetes.

According to some embodiments of the invention, the diabetes-associated autoantigenic peptide is GAD₅₅₅₋₅₆₇ (NFFRMVISNPAAT; SEQ ID NO:4).

Tables 3, 4 and 5, hereinbelow, provide non-limiting examples of MHC class II restricted diabetes associated autoantigens which can form a complex with an MHC class II molecule according to some embodiments of the invention.

TABLE 3
GAD, ZnT8 and IA-2 derived autoantigenic peptides

| SEQ | | | SEQ | | | SEQ | | |
|---|---|---|---|---|---|---|---|---|
| ID | | | ID | | | ID | | |
| NO: | GAD | MHC | NO: | ZnT8 | MHC | NO: | IA-2 | MHC |
| 256 | MNILLQYV | DR4 | 304 | LTIQIES | DQ8 | 312 | VSSVSSQ | |
| | VKSFD | | | AADQDPS | | | FSDAAQA | DR4 |
| | | | | | | | SPSSFSD | |
| 257 | IAPVFVLLE | DR4 | 305 | RTGIAQ | DQ8 | 313 | LAKEWQ | DR4 |
| | | | | ALSSFD | | | ALCAYQ | |
| | | | | LH | | | AEPNTCA | |
| | | | | | | | TAQGE | |
| 258 | LPRLIAFTS | DR4 | 306 | LYPDYQ | DQ8 | 314 | KLKVESS | DR4 |
| | EHSHF | | | IQAGIMIT | | | PSRSDYIN | |
| | | | | | | | ASPIIEHDP | |
| 259 | IAFTSEHSH | DR4 | 307 | ILSVHV | DQ8 | 315 | IKLKVESS | DR4 |
| | FSLK | | | ATAASQ | | | PSRSDYIN | |
| | | | | DS | | | ASPI | |
| 260 | TVYGAFDP | DR4 | 308 | SKRLTF | DQ8 | 316 | MVWESG | DR4 |
| | LLAVAD | | | GWYRA | | | CTVIVML | |
| | | | | EIL | | | TPLVEDGV | |
| 261 | KYKIWMH | DR4 | 309 | AILTDA | DQ8 | 317 | RQHARQ | DQ8 |
| | VDAAWGGG | | | AHLLID | | | QDKERLA | |
| | | | | LT | | | ALGPE | |
| 262 | KHKWKLS | DR4 | 310 | KATGNR | DQ8 | 318 | GPEGAHG | DQ8 |
| | GVERANSV | | | SSKQAH | | | DTTFEYQ | |
| | | | | AK | | | DLCR | |
| 263 | LYNIIKNRE | DR4 | 311 | AVDGVI | DQ8 | 319 | EGPPEPSR | DQ8 |
| | GYEMVF | | | SVHSLHIW | | | VSSVSSQ | |
| | | | | | | | FSD | |
| 264 | PSLRTLED | DR4 | | | | 320 | FSDAAQA | DQ8 |
| | NEERMSR | | | | | | SPSSHSST | |
| | | | | | | | PSW | |
| 265 | RMMEYGT | DR4 | | | | 321 | AEPNTCA | DQ8 |
| | TMVSYQPL | | | | | | TAQGEGN | |
| | | | | | | | IKKN | |
| 266 | SYQPLGDK | DR4 | | | | 322 | NASPIIEH | DQ8 |
| | VNFFRMV | | | | | | DPRMPAY | |
| | | | | | | | IAT | |
| 267 | NFFRMVIS | DR4 | | | | 323 | DEGSALY | DQ8 |
| | NPAAT | | | | | | HVYEVNL | |
| | | | | | | | VSEH | |
| 268 | ATHQDIDF | DR4 | | | | 324 | KGVKEID | DQ8 |
| | LIEEIER | | | | | | IAATLEH | |
| | | | | | | | VRDO | |
| 269 | ATDLLPACD | DQ8 | | | | 325 | FALTAVA | DQ8 |
| | | | | | | | EEVNAIL | |
| | | | | | | | KALPQ | |
| 270 | FDRSTKVI | DQ8 | | | | 326 | KNRSLAV | DQ8 |
| | DFHYPNE | | | | | | LTYDHSRI | |
| 271 | ELLQEYN | DQ8 | | | | 327 | GADPSAD | DQ8 |
| | WE | | | | | | ATEAYQEL | |
| 272 | EYNWELA | DQ8 | | | | 328 | EIDIAATLE | DQ8 |
| | DQ | | | | | | | |
| 273 | DIDFLIEEI | DQ8 | | | | 329 | NTCATAQ | DQ8 |
| | | | | | | | GE | |
| 274 | TGHPRYFN | DQ8 | | | | 330 | EPNTCAT | DQ8 |
| | QLSTGLD | | | | | | AQ | |
| 275 | TYEIAPVF | DQ8 | | | | 331 | ERLAALG | DQ8 |
| | VLLEYVT | | | | | | PE | |
| 276 | YVTLKKM | DQ8 | | | | 332 | QHARQQ | DQ8 |
| | RE | | | | | | DKE | |
| 277 | PGGSGDGI | DQ8 | | | | 333 | YEVNLVS | DQ8 |
| | FSPGGAISN | | | | | | EH | |
| | MYA | | | | | | | |
| 278 | NMYAMMI | DQ8 | | | | 334 | GASLYHV | DQ8 |
| | ARFKMFPE | | | | | | YE | |
| | VKEKG | | | | | | | |
| 279 | PEVKEKG | DQ8 | | | | 335 | FALTAVA | DQ8 |
| | MAALPRLI | | | | | | EE | |
| | AFTSE | | | | | | | |
| 280 | DSVILIKCD | DQ8 | | | | 336 | GAHGDTT | DQ8 |
| | | | | | | | FE | |
| 281 | GKMIPSDLE | DQ8 | | | | 337 | GDTTFEY | DQ8 |
| | | | | | | | QD | |
| 282 | ERRILEAKQ | DQ8 | | | | 338 | AAQASPS | DQ8 |
| | | | | | | | SH | |
| 283 | ERANSVT | DQ8 | | | | 339 | SRVSSVS | DQ8 |
| | WN | | | | | | SQ | |
| 284 | QCSALLVRE | DQ8 | | | | 340 | TQFHFLS | DQ8 |
| | | | | | | | WP | |
| 285 | KHYDLSYD | DQ8 | | | | 341 | EEPAQAN | DQ8 |
| | TGDKALQ | | | | | | MD | |
| 286 | AKGTTGFE | DQ | | | | 342 | GHMILAY | DQ8 |
| | AHVDKCL | | | | | | ME | |
| 287 | VDKCLELA | DQ8 | | | | 343 | MILAYME | DQ8 |
| | EYLYNIIKN | | | | | | DH | |
| | REG | | | | | | | |
| 288 | IIKNREGYE | DQ8 | | | | 344 | QALCAY | DQ8 |
| | | | | | | | QAE | |
| 289 | MVFDGKP | DQ8 | | | | 345 | EWQALC | DQ8 |
| | QHTNVCFW | | | | | | AYQ | |
| 290 | CFWYIPPSL | DQ8 | | | | 346 | LVRSKDQ | DQ8 |
| | RTLEDN | | | | | | FE | |
| 291 | FWYIPPSLR | DQ8 | | | | 347 | VEDGVK | DQ8 |
| | TLED | | | | | | QCD | |
| 292 | SLRTLEDNE | DQ8 | | | | 348 | YILIDMV | DQ8 |
| | | | | | | | LN | |
| 293 | ERMSRLSK | DQ8 | | | | 349 | ESGCTVI | DQ8 |
| | VAPVIKA | | | | | | VM | |
| 294 | IKARMME | DQ8 | | | | 350 | LCAYQAE | DQ8 |
| | YGTTMVSY | | | | | | PN | |
| 295 | RMMEYGT | DQ8 | | | | 351 | ETRTLTQ | DQ8 |
| | TMVSYQPL | | | | | | FH | |
| 296 | VISNPAATH | DQ8 | | | | 352 | VESSPSRSD | DQ8 |
| 297 | IDFLIEEIE | DQ8 | | | | 353 | GPLSHTIAD | DQ8 |
| 298 | NWELADQ | DR2 | | | | 354 | SLFNRAE | DQ8 |
| | PQNLEEIL | | | | | | GP | |
| | MHCQT | | | | | | | |
| 299 | GHPRYFNQ | DR2 | | | | 355 | HPDFLPY | DQ8 |
| | LSTG | | | | | | DH | |
| 300 | TYEIAPVF | DR2 | | | | 356 | HFLSWPA | DQ8 |
| | VLLFYVTL | | | | | | EG | |
| | KKMR | | | | | | | |
| 301 | VNFFRMVI | DR4 | | | | 357 | DFRRKVN | DQ8 |
| | SNPAATHQD | | | | | | KC | |
| 302 | DKVNFFR | DR4 | | | | 358 | HCSDGAG | DQ8 |
| | MVISNPAA | | | | | | RT | |
| | THQDID | | | | | | | |
| 303 | FFRMVISN | core | | | | 359 | LVRSFYL | DQ8 |
| | PA | sequence | | | | | KN | |
| | | | | | | 360 | KNRSLAV | DQ8 |
| | | | | | | | LTYDHSRI | |
| | | | | | | 361 | GADPSAD | DQ8 |
| | | | | | | | ATEAYQEL | |
| | | | | | | 362 | ANMDIST | Unknown |
| | | | | | | | GHMILAY | |
| | | | | | | | ME | |
| | | | | | | 363 | WQALCA | unknown |
| | | | | | | | YQAEPNT | |
| | | | | | | | CAT | |
| | | | | | | 364 | LSHTIADF | unknown |
| | | | | | | | WQMVWE | |
| | | | | | | | SG | |
| | | | | | | 365 | DFWQMV | unknown |
| | | | | | | | WESGCTV | |
| | | | | | | | IVM | |
| | | | | | | 366 | WESGCTV | unknown |
| | | | | | | | IVMLTPL | |
| | | | | | | | VE | |
| | | | | | | 367 | VIVMLTP | unknown |
| | | | | | | | LVEDGVK | |
| | | | | | | | QC | |
| | | | | | | 368 | SEHIWCE | unknown |
| | | | | | | | DFLVRSF | |
| | | | | | | | YL | |
| | | | | | | 369 | WCEDFLV | unknown |
| | | | | | | | RSFYLKN | |
| | | | | | | | VQ | |
| | | | | | | 370 | EDFLVRS | unknown |
| | | | | | | | FYLKNVQ | |
| | | | | | | | TQ | |
| | | | | | | 371 | DFRRKVN | unknown |
| | | | | | | | KCYRGRS | |
| | | | | | | | CP | |
| | | | | | | 372 | YILIDMV | unknown |
| | | | | | | | LNRMAK | |
| | | | | | | | GVK | |
| | | | | | | 373 | FEFALTA | unknown |
| | | | | | | | VAEEVNA | |
| | | | | | | | IL | |

Table 3 Provided are diabetes-associated autoantigenic peptides (with their sequence identifiers, SEQ ID NO:) and the MHC class II molecules which bind thereto.

TABLE 4
Preproinsulin and HSP-60 autoantigenic peptide

| SEQ | | | SEQ | | |
|---|---|---|---|---|---|
| ID | | | ID | | |
| NO: | PREPROINSULIN | MHC | NO: | HSP-60 | MHC |
| 374 | EALYLVCGE | DQ8 | 395 | KFGADARALML | Unknown |
| | | | | QGVDLLADA | |
| 375 | SICSLYQLE | DQ8 | 396 | NPVEIRRGVMLA | Unknown |
| | | | | VDAVIAEL | |
| 376 | ALLALWGPD | DQ8 | 397 | QSIVPALEIANAH | Unknown |
| | | | | RKPLVIIA | |
| 377 | GSLQPLALE | DQ8 | 398 | LVLNRLKVGLQ | Unknown |
| | | | | VVAVKAPGF | |
| 378 | TPKTRREAE | DQ8 | 399 | IVLGGGCALLRCI | Unknown |
| | | | | PALDSLT | |
| 379 | PAAAFVNQH | DQ8 | 400 | VLGGGCALLRCI | Unknown |
| | | | | PALDSLTPANED | |
| 380 | DPAAAFVNQ | DQ8 | 401 | EIIKRTLKIPAMTI | Unknown |
| | | | | AKNAGV | |
| 381 | PDPAAAFVN | DQ8 | 402 | VNMVEKGIIDPT | Unknown |
| | | | | KVVRTALL | |
| 382 | QKRGIVEQC | DQ8 | | | |
| 383 | ELGGGPGAG | DQ8 | | | |
| 384 | EAEDLQVGQ | DQ8 | | | |
| 385 | LQVGQVELG | DQ8 | | | |
| 386 | HLCGSHLVE | DQ8 | | | |
| 387 | GIVEQCCTSICS | DR4 | | | |
| 388 | KRGIVEQCCT | DR4 | | | |
| | SICS | | | | |
| 389 | LALLALWGP | Unknown | | | |
| | DPAAAFV | | | | |
| 390 | PAAAFVNQH | Unknown | | | |
| | LCGSHLV | | | | |
| 391 | SHLVEALYLV | Unknown | | | |
| | CGERG | | | | |
| 392 | FFYTPKTRRE | Unknown | | | |
| | AED | | | | |
| 393 | GAGSLQPLAL | Unknown | | | |
| | EGSLQKRG | | | | |
| 394 | SLQKRGIVEQ | Unknown | | | |
| | CCTSICS | | | | |

Table 4. Provided are the diabetes-associated autoantigenic peptides (with their sequence identifiers, SEQ ID NO:) and the MHC class II molecules which bind thereto.

TABLE 5
HSP-70 and IGRP derived autoantigenic peptides

| SEQ | | | | | |
|---|---|---|---|---|---|
| ID | | | SEQ ID | | |
| NO: | HSP-70 | MHC | NO: | IGRP | MHC |
| 403 | MAKAAAVGIDLGTT | Unknown | 412 | QHLQKDYRAY | DR3 |
| | YSCVGV | | | YTF | |
| 404 | GLNVLRIINEPTAAAI | Unknown | 413 | RVLNIDLLWSV | DR3 |
| | AYGL | | | PI | |
| 405 | TIDDGIFEVKATAGD | Unknown | 414 | YTFLNFMSNV | DR4 |
| | THLGG | | | GDP | |
| 406 | THLGGEDFDNRLVN | Unknown | 415 | DWIHIDTTPFA | DR4 |
| | HFVEEF | | | GL | |
| 407 | KRTLSSSTQASLEIDS | Unknown | | | |
| | LFEG | | | | |
| 408 | LLLLDVAPLSLGLET | Unknown | | | |
| | AGGVM | | | | |
| 409 | PTKQTQIFTTYSDNQP | Unknown | | | |
| | GVLI | | | | |
| 410 | KANKITITNDKGRLS | Unknown | | | |
| | KEEIE | | | | |
| 411 | KEEIERMVQEAEKYK | Unknown | | | |
| | AEDEV | | | | |

Table 5. Provided are the diabetes-associated autoantigenic peptides (with their sequence identifiers, SEQ ID NO:) and the MHC class II molecules which bind thereto.

Further description of type I diabetes-associated autoantigenic peptides can be found in Lieberman S M, DiLorenzo T P, 2003. A comprehensive guide to antibody and T-cell responses in type 1 diabetes. Tissue Antigens, 62:359-77; Liu J, Purdy L E, Rabinovitch S, Jevnikar A M, Elliott J F. 1999, Major DQ8-restricted T-cell epitopes for human GAD65 mapped using human CD4, DQA1*0301, DQB1*0302 transgenic IA(null) NOD mice, Diabetes, 48: 469-77; Di Lorenzo T P, Peakman M, Roep B O. 2007, Translational mini-review series on type 1 diabetes: Systematic analysis of T cell epitopes in autoimmune diabetes. Clin Exp Immunol. 148:1-16; Stadinski et al Immunity 32:446, 2010; each of which is fully incorporated herein by reference).

According to some embodiments of the invention, the GAD autoantigenic peptide comprises a core amino acid sequence set forth by SEQ ID NO: 5 (GAD₅₅₆₋₅₆₅, FFRMVISNPA).

According to some embodiments of the invention, the GAD autoantigenic peptide comprises a core amino acid sequence set forth by SEQ ID NO: 5 (GAD₅₅₆₋₅₆₅, FFRMVISNPA) and no more than 30 amino acids.

According to some embodiments of the invention, the GAD autoantigenic peptide is GAD₅₅₅₋₅₆₇ (NFFRMVISNPAAT; SEQ ID NO:4).

According to some embodiments of the invention, the diabetes-associated autoantigenic peptide is a GAD derived autoantigenic peptide selected from the group consisting of SEQ ID NOs:256-303

According to some embodiments of the invention, the diabetes-associated autoantigenic peptide is a ZnT8 derived autoantigenic peptide selected from the group consisting of SEQ ID NOs: 304-311.

According to some embodiments of the invention, the diabetes-associated autoantigenic peptide is a IA-2 derived autoantigenic peptide selected from the group consisting of SEQ ID NOs: 312-373.

According to some embodiments of the invention, the diabetes-associated autoantigenic peptide is a preproinsulin derived autoantigenic peptide selected from the group consisting of SEQ ID NOs:374-394.

According to some embodiments of the invention, the diabetes-associated autoantigenic peptide is a HSP-60 derived autoantigenic peptide selected from the group consisting of SEQ ID NOs: 395-402.

According to some embodiments of the invention, the diabetes-associated autoantigenic peptide is a HSP-70 derived autoantigenic peptide selected from the group consisting of SEQ ID NOs: 403-411.

According to some embodiments of the invention, the diabetes-associated autoantigenic peptide is a IGRP derived autoantigenic peptide selected from the group consisting of SEQ ID NOs:412-415.

According to some embodiments of the invention, the multiple sclerosis-associated autoantigenic peptide is derived from a polypeptide selected from the group consisting of myelin oligodendrocyte glycoprotein [MOG; GenBank Accession Nos. NP_001008229.1 (SEQ ID NO: 54); NP_001008230.1 (SEQ ID NO: 55); NP_001163889 (SEQ ID NO: 56); NP_002424.3 (SEQ ID NO: 57); NP_996532 (SEQ ID NO: 58); NP_996533.2 (SEQ ID NO: 59); NP_996534.2 (SEQ ID NO: 60); NP_996535.2 (SEQ ID NO: 61); NP_996537.3 (SEQ ID NO: 62)], myelin basic protein [MBP; GenBank Accession Nos. NP_001020252.1 (SEQ ID NO: 63); NP_001020261.1 (SEQ ID NO: 64); NP_001020263.1 (SEQ ID NO: 65); NP_001020271.1 (SEQ ID NO: 66); NP_001020272.1 (SEQ ID NO: 67); NP_002376.1 (SEQ ID NO: 68)], and proteolipid protein [PLP1; GenBank Accession Nos. NP_000524.3 (SEQ ID NO: 69); NP_001122306.1 (SEQ ID NO: 70); NP_955772.1 (SEQ ID NO: 71)].

Tables 6 and 7, hereinbelow, provide non-limiting examples of MHC class II restricted multiple sclerosis associated autoantigens which can form a complex with an MHC class II molecule according to some embodiments of the invention.

TABLE 6
Multiple sclerosis associated autoantigens derived
from Myelin basic protein (MBP) and ProteoLipid
Protein (PLP)

| SEQ ID | MBP (Myelin basic | | SEQ ID | PLP (Proteo Lipid | |
|---|---|---|---|---|---|
| NO: | protein) | MHC | NO: | protein) | MHC |
| 416 | RSQPGLCNMYKDSHHP | unknown | 424 | VFACSAVPVYI | unknown |
| | ARTA | | | YFNTWTTCQS | |
| 417 | FKGVDAQGTLSKIFKLG | unknown | 425 | YIYFNTWTTCQ | unknown |
| | GRDS | | | SIAFPSKTSA | |
| 418 | GDRGAPKRGSGKVPWL | DP | 426 | AHSLERVCHCL | DR |
| | KPGRS | | | GKWLGHPDKF | |
| 419 | RSQPGLCNMYKDSHHP | DP | 427 | AVRQIFGDYKT | DR |
| | ARTA | | | TICGKGLSAT | |
| 420 | SDYKSAHKGFKGVDAQ | DR | 428 | FMIAATYNFAV | DR |
| | GTLSK | | | LKLMGRGTKF | |
| 421 | FKGVDAQGTLSKIFKLG | DR | 429 | AHSLERVCHCL | DQ |
| | GRDS | | | GKWLGHPDKF | |
| 422 | SDYKSAHKGFKGVDAQ | DQ | 430 | AVRQIFGDYKT | DQ |
| | GTLSK | | | TICGKGLSAT | |
| 423 | ENPVVHFFKNIVTPR | DR2 | 431 | CQSIAFPSKTSA | DQ |
| | | | | SIGSLCAD | |
| | | | 432 | SKTSASIGSLC | DQ |
| | | | | ADARMYGVL | |
| | | | 433 | GVLPWNAFPG | DQ |
| | | | | KVCGSNLLSI | |
| | | | 434 | FMIAATYNFAV | DQ |
| | | | | LKLMGRGTKF | |

Table 6.

TABLE 7
Multiple sclerosis associated autoantigens
derived from Myelin Oligodendrocyte
Glycoprotein (MOG)

| MHC | SEQ ID NO: | Peptide |
|---|---|---|
| unknown | 435 | ELKVEDPFYWVSPGVLVLLAV |
| unknown | 436 | TFDPHFLRVPCWKITLFVIV |
| unknown | 437 | VIVPVLGPLVALIICYNWLHR |
| unknown | 438 | VALIICYNWLHRRLAGQFLEE |
| DR | 439 | GFTCFFRDHSYQEEAAMELKV |
| DR | 440 | ITVGLVFLCLQYRLRGKLRAE |
| DR | 441 | VALIICYNWLHRRLAGQFLEE |
| DR | 442 | LQYRLRGKLRAEIENLHRTFD |
| DQ | 443 | ELKVEDPFYWVSPGVLVLLAV |
| DQ | 444 | LQYRLRGKLRAEIENLHRTFD |
| DR2 | 472 | MEVGWYRPPFSRVVHLYRNGK |
| DR2 | 445 | PERYGRTELLKDAIGEGKVTLRIRN |
| DR4 | 446 | TCFFRDHSYQEE |
| DR4 | 447 | FVIVPVLGP |
| DR4 | 448 | KITLFVIVPVLGP |

Table 7.

According to some embodiments of the invention, the MOG autoantigenic peptide is MOG-35-55 (SEQ ID NO: 6; MEVGWYRPPFSRVVHLYRNGK).

According to some embodiments of the invention, the MBP autoantigenic peptide is MBP-85-99 (SEQ ID NO: 22).

According to some embodiments of the invention, the rheumatoid arthritis-associated autoantigenic peptide is derived from a polypeptide selected from the group consisting of: Collagen II (COL2A1, GenBank Accession NO. NP_001835.3; SEQ ID NO: 81), Matrix metalloproteinase-1 (MMP1) [GenBank Accession NO. NP_001139410.1 (SEQ ID NO:72); and GenBank Accession NO. NP_002412.1 (SEQ ID NO:73)], Aggrecan Core Protein Precursor (ACAN) [GenBank Accession NO. NP_001126.3 (SEQ ID NO:74); and GenBank Accession NO. NP_037359.3 (SEQ ID NO:75)], Matrix Metalloproteinase-16 (MMP16) [GenBank Accession NO. NP_005932.2 (SEQ ID NO:76)], Tenascin (TNXB) [GenBank Accession NO.

NP_061978.6 (SEQ ID NO:77) and GenBank Accession NO. NP_115859.2 (SEQ ID NO:78)] and Heterogeneous Nuclear Ribonucleoprotein A2 (HNRNPA2B1) [GenBank Accession NO. XP_005249786.1 (SEQ ID NO:79) and GenBank Accession NO. XP_006715777.1 (SEQ ID NO:80)].

Tables 8-12, hereinbelow, provide non-limiting examples of MHC class II restricted rheumatoid arthritis associated autoantigens which can form a complex with an MHC class II molecule according to some embodiments of the invention.

TABLE 8
Rheumatoid arthritis associated Collagen II and
Matrix metalloproteinase-1 autoantigens

| | SEQ | | | SEQ | Matrix |
|---|---|---|---|---|---|
| | ID | collagen II autoantigenic | | ID | metalloproteinase-1 |
| MHC | NO: | peptide | MHC | NO: | autoantigenic peptide |
| DR4/ | 449 | AGFKGEQGPKGEP | unknown | 451 | GVVSHSFPATLETQE |
| DR1 | | | | | |
| DR4/ | 450 | EPGIAGFKGEQGPKGEPG | unknown | | |
| DR1 | | | | | |

Table 8.

TABLE 9
Rheumatoid arthritis associated Aggrecan core protein
precursor and Matrix metalloproteinase-3 autoantigens

| | | | | | MATRIX |
|---|---|---|---|---|---|
| | SEQ | AGGRECAN CORE PROTEIN | | SEQ | METALLOPROTEINASE-3 |
| | ID | PRECURSOR | | ID | AUTOANTIGENIC |
| MHC | NO: | AUTOANTIGENIC PEPTIDE | MHC | NO: | PEPTIDE |
| unknown | 452 | LSGLPSGGEVLEISV | unknown | 454 | FFYFFTGSSQLEFDP |
| unknown | 453 | ISGLPSGGDDLETST | | | |

Table 9.

TABLE 10
Rheumatoid arthritis associated Calpain-2 and
Matrix metalloproteinase-10 autoantigens

| | SEQ | | | SEQ | Matrix |
|---|---|---|---|---|---|
| | ID | Calpain-2 autoantigenic | | ID | metalloproteinase-10 |
| MHC | NO: | peptide | MHC | NO: | autoantigenic peptide |
| unknown | 455 | HAYSVTGAEEVESNG | unknown | 456 | SAFWPSLPSGLDAAY |

Table 10.

TABLE 11
Rheumatoid arthritis associated Fibrillin-1 precursor
and Matrix metalloproteinase-16 autoantigens

| | SEQ | | | SEQ | Matrix |
|---|---|---|---|---|---|
| | ID | Fibrillin-1 precursor | | ID | metalloproteinase-16 |
| MHC | NO: | autoantigenic peptide | MHC | NO: | autoantigenic peptide |
| unknown | 457 | CVDTRSGNCYLDIRP | unknown | 458 | VKEGHSPPDDVDIVI |

Table 11.

TABLE 12
Rheumatoid arthritis associated Tenascin and heterogeneous
nuclear ribonucleoprotein A2 autoantigens

| | SEQ | | | SEQ | Heterogeneous nuclear |
|---|---|---|---|---|---|
| | ID | Tenascin autoantigenic | | ID | ribonucleoprotein A2 |
| MHC | NO: | peptide | MHC | NO: | autoantigenic peptide |
| unknown | 459 | EPVSGSFTTALDGPS | DR1/DR4 | 460 | RDYFEEYGKIDTIEIIT |

Table 12.

According to some embodiments of the invention, the celiac-associated autoantigenic peptide is derived from alpha Gliadin [e.g., GenBank Accession Nos. ADM96154 (SEQ ID NO: 82), ADD17013.1 (SEQ ID NO: 83)], gamma Gliadin [e.g., from Aegilops tauschii GenBank Accession No. CAC10631.1, SEQ ID NO: 84] and Heat shock 20 [GenBank Accession No. AAB81196 (SEQ ID NO: 85)].

Tables 13 and 14, hereinbelow, provide a non-limiting list of MHC class II restricted celiac associated autoantigens which can form a complex with an MHC class II molecule according to some embodiments of the invention.

TABLE 13
Celiac associated gliadin autoantigens

| | SEQ | | | SEQ | |
|---|---|---|---|---|---|
| | ID | | | ID | α-gliadin |
| MHC | NO: | α-Gliadin autoantigenic peptide | MHC | NO: | autoantigenic peptide |
| DQ2/DQ8 | 461 | LGQQQPFPPQQPY | DQ2 | 462 | QLQPFPQPQLPY |
| DQ2/DQ8 | 473 | FPQPELPYPQP (Gliadin-61-71) | DQ2 | 463 | PQPQLPYPQPQLPY |
| DQ2/DQ8 | 474 | VPVPQLQPQNPSQQQPQEQVPL | DQ2 | 464 | PGQQQPFPPQQPY |
| | | (Gliadin-3-24) | | | |

Table 13.

TABLE 14
Celiac associated γ-gliadin and heat shock 20 autoantigens

| | | | | | Heat shock 20 |
|---|---|---|---|---|---|
| | SEQ | | | SEQ | autoantigenic |
| MHC | ID NO: | γ-Gliadin autoantigenic peptide | MHC | ID NO: | peptide |
| DQ2 | 465 | GIIQPQQPAQL | unknown | 470 | ALPTAQVPTDP |
| DQ2 | 466 | FPQQPQQPYPQQP | unknown | 471 | GRLFDQRFGEG |
| DQ2 | 467 | FSQPQQQFPQPQ | | | |
| DQ2 | 468 | PQQPFPQQPQQPY | | | |
| DQ2 | 469 | FLQPQQPFPQQPQQPYPQQPQQPFPQ | | | |

Table 14.

According to some embodiments of the invention, the stroke-associated autoantigenic peptide is derived from a brain antigen such as myelin basic protein, neurofilaments and the NR2A/2B subtype of the N-methyl-D-aspartate receptor (MOG-35-55-MEVGWYRPPFSRVVHLYRNGK (SEQ ID NO: 6)).

Since the amino acid sequence of the autoantigen may vary in length between the same or different MHC class II alleles, the length of the autoantigenic peptides according to some embodiments of the invention may vary from at least 6 amino acids, to autoantigenic peptides having at least 8, 10, 25, or up to 30 amino acids.

According to some embodiments of the invention, the autoantigenic peptide includes a core amino acids of at least 6 amino acids, e.g., at least 7, at least 8, at least 9 and more.

According to some embodiments of the invention, the length of the autoantigenic peptide does not exceed about 100 amino acids, e.g., does not exceed about 50 amino acids, e.g., does not exceed about 30 amino acids.

According to some embodiments of the invention, the length of the autoantigenic peptide includes at least 6 and no more than 30 amino acids.

In addition, it should be noted that although some amino acids in each autoantigenic peptide are conserved between various alleles of MHC class II and cannot be substituted, other amino acids can be substituted with amino acids having essentially equivalent specificity and/or affinity of binding to MHC molecules and resulting in equivalent T cell epitope as the amino acid sequences shown in the exemplary autoantigens described above. Thus, in each autoantigenic peptide there are at least six amino acids constituting a core amino acid which are required for recognition with the respective MHC class II molecule. Identification of the core amino acids for each autoantigenic peptide can be done experimentally, e.g., by mutagenesis of the amino acids constituting the autoantigenic peptide and detection of: (i) binding to the restricted MHC class II molecules; (ii) Stimulating the restricted T cell response. The core amino acid sequence consists of anchor residues and the T-cell receptor (TCR) contact residues. For example, for the GAD autoantigenic peptide the anchor residues in the sequence NFFRMVISNPAAT (SEQ ID NO: 32) are the P1 (F557), P4 (V560), P6 (S562), and P9 (A565) MHC pocket-binding residues. TCR contact residues in the sequence NFFRMVISNPAAT (SEQ ID NO: 33) are at positions F556, R558, M559, I561, N563. Accordingly, the core amino acids of the GAD555-567 autoantigenic peptide are GAD556-565 (FFRMVISNPA, SEQ ID NO: 5).

The invention according to some embodiments thereof also concerns peptide variants whose sequences do not completely correspond with the aforementioned amino acid sequences but which only have identical or closely related “anchor positions”. The term “anchor position” in this connection denotes an essential amino acid residue for binding to a MHC class II complex (e.g., DR1, DR2, DR3, DR4 or DQ). The anchor position for the DRB1*0401 binding motif are for example stated in Hammer et al., Cell 74 (1993), 197-203. Such anchor positions are conserved in the autoantigenic peptide or are optionally replaced by amino acid residues with chemically very closely related side chains (e.g. alanine by valine, leucine by isoleucine and visa versa). The anchor position in the peptides according to some embodiments of the invention can be determined in a simple manner by testing variants of the aforementioned specific peptides for their binding ability to MHC molecules. Peptides according to some embodiments of the invention are characterized in that they have an essentially equivalent specificity or/and affinity of binding to MHC molecules as the aforementioned peptides. Homologous peptides having at least 50%, e.g., at least 60%, 70%, 80%, 90%, 95% or more identity to the autoantigenic peptides described herein are also contemplated by some embodiments of the invention.

According to some embodiments of the invention, the antigen is a non-MHC restricted antigen.

Table 15 below provides a non-limiting list of non-MHC restricted antigens which can be used according to some embodiments of the invention.

TABLE 15
Tumors antigens and CARs in in vitro and in vivo trials

| | | SEQ | | | |
|---|---|---|---|---|---|
| | GenBank Accession | ID | Associated | | CARs |
| Target antigen | No. | NO: | malignancy | Receptor type | generation |
| α-Folate receptor | folate receptor 1 | 150-154 | Ovarian cancer | ScFv- | First |
| | (FOLR1) GenBank | | | FcεRIγCAIX | |
| | Accession Nos. | | | | |
| | NP_000793.1; | | | | |
| | NP_057936.1; | | | | |
| | NP_057937.1; | | | | |
| | NP_057941.1 | | | | |
| CAIX | carbonic anhydrase | 155 | Renal cell | ScFv-FcεRIγ | First |
| | IX GenBank | | carcinoma | | |
| | Accession No._{—} | | | | |
| | NP_001207.2 | | | | |
| CD19 | CD19 molecule | 156-157 | B-cell malignancies | ScFv-CD3ζ | First |
| | GenBank Accession | | | (EBV) | |
| | Nos._{—} | | | | |
| | NP_001171569.1; | | | | |
| | NP_001761.3 | | | | |
| CD19 | | | B-cell malignancies, | ScFv-CD3ζ | First |
| | | | CLL | | |
| CD19 | | | B-ALL | ScFv-CD28- | Second |
| | | | | CD3ζ | |
| CD19 | | | ALL | CD3ζ(EBV) | First |
| CD19 | | | ALL post-HSCT | ScFv-CD28- | Second |
| | | | | CD3ζ | |
| CD19 | | | Leukemia, | ScFv-CD28- | First and |
| | | | lymphoma, CLL | CD3ζ vs. | Second |
| | | | | CD3ζ | |
| CD19 | | | B-cell malignancies | ScFv-CD28- | Second |
| | | | | CD3ζ | |
| CD19 | | | B-cell malignancies | ScFv-CD28- | Second |
| | | | post-HSCT | CD3ζ | |
| CD19 | | | Refractory | ScFv-CD3ζ | First |
| | | | Follicular | | |
| | | | Lymphoma | | |
| CD19 | | | B-NHL | ScFv-CD3ζ | First |
| CD19 | | | B-lineage lymphoid | ScFv-CD28- | Second |
| | | | malignancies post- | CD3ζ | |
| | | | UCBT | | |
| CD19 | | | CLL, B-NHL | ScFv-CD28- | Second |
| | | | | CD3ζ | |
| CD19 | | | B-cell malignancies, | ScFv-CD28- | Second |
| | | | CLL, B-NHL | CD3ζ | |
| CD19 | | | ALL, lymphoma | ScFv-41BB- | First and |
| | | | | CD3ζ vs | Second |
| | | | | CD3ζ | |
| CD19 | | | ALL | ScFv-41BB- | Second |
| | | | | CD3ζ | |
| CD19 | | | B-cell malignancies | ScFv-CD3ζ | First |
| | | | | (Influenza | |
| | | | | MP-1) | |
| CD19 | | | B-cell malignancies | ScFv-CD3ζ | First |
| | | | | (VZV) | |
| CD20 | membrane-spanning | 158-159 | Lymphomas | ScFv-CD28- | Second |
| | 4-domains, | | | CD3ζ | |
| | subfamily A, | | | | |
| | member 1 (MS4A1, | | | | |
| | CD20) GenBank | | | | |
| | Accession Nos. | | | | |
| | NP_068769.2; | | | | |
| | NP_690605.1 | | | | |
| CD20 | | | B-cell malignancies | ScFv-CD4- | Second |
| | | | | CD3ζ | |
| CD20 | | | B-cell lymphomas | ScFv-CD3ζ | First |
| CD20 | | | Mantle cell | ScFv-CD3ζ | First |
| | | | lymphoma | | |
| CD20 | | | Mantle cell | CD3 ζ/ | Third |
| | | | lymphoma, indolent | CD137/CD28 | |
| | | | B-NHL | | |
| CD20 | | | indolent B cell | ScFv-CD28- | Second |
| | | | lymphomas | CD3ζ | |
| CD20 | | | Indolent B cell | ScFv-CD28- | Third |
| | | | lymphomas | 41BB-CD3ζ | |
| CD22 | CD22 molecule | 160-164 | B-cell malignancies | SFV-CD4- | Second |
| | GenBank Accession | | | CD3ζ | |
| | Nos. | | | | |
| | NP_001172028.1; | | | | |
| | NP_001172029.1; | | | | |
| | NP_001172030.1; | | | | |
| | NP_001265346.1; | | | | |
| | NP_001762.2; | | | | |
| CD30 | tumor necrosis factor | 165-166 | Lymphomas | ScFv-FcεRIγ | First |
| | receptor superfamily, | | | | |
| | member 8 | | | | |
| | (TNFRSF8; CD30); | | | | |
| | GenBank Accession | | | | |
| | Nos. NP_001234.3; | | | | |
| | NP_001268359.2; | | | | |
| CD30 | | | Hodgkin lymphoma | ScFv-CD3ζ | First |
| | | | | (EBV) | |
| CD33 | CD33 molecule | 167-169; | AML | ScFv-CD28- | Second |
| | GenBank Accession | | | CD3ζ | |
| | Nos. | | | | |
| | NP_001076087.1; | | | | |
| | NP_001171079.1; | | | | |
| | NP_001763.3 | | | | |
| CD33 | | | AML | ScFv-41BB- | Second |
| | | | | CD3ζ | |
| CD44v7/8 | CD44 molecule | 170-177 | Cervical carcinoma | ScFv-CD8- | Second |
| | (Indian blood group) | | | CD3ζ | |
| | GenBank Accession | | | | |
| | Nos. NP_000601.3; | | | | |
| | NP_001001389.1; | | | | |
| | NP_001001390.1; | | | | |
| | NP_001001391.1; | | | | |
| | NP_001001392.1; | | | | |
| | NP_001189484.1; | | | | |
| | NP_001189485.1; | | | | |
| | NP_001189486.1; | | | | |
| CEA | | | Breast cancer | ScFv-CD28- | Second |
| | | | | CD3ζ | |
| CEA | | | Colorectal cancer | ScFv-CD3ζ | First |
| CEA | | | Colorectal cancer | ScFv-FceRIγ | First |
| CEA | | | Colorectal cancer | ScFv-CD3ζ | First |
| CEA | | | Colorectal cancer | ScFv-CD28- | Second |
| | | | | CD3ζ | |
| CEA | | | Colorectal cancer | ScFv-CD28- | Second |
| | | | | CD3ζ | |
| EGP-2 | epithelial cell | 178 | Multiple | scFv-CD3ζ | First |
| | adhesion molecule | | malignancies | | |
| | (EPCAM) GenBank | | | | |
| | Accession No. | | | | |
| | NP_002345.2 | | | | |
| EGP-2 | | | Multiple | scFv-FcεRIγ | First |
| | | | malignancies | | |
| EGP-40 | epithelial cell | 178 | Colorectal cancer | scFv-FcεRIγ | First |
| | adhesion molecule | | | | |
| | (EPCAM); GenBank | | | | |
| | Accession No. | | | | |
| | NP_002345.2 | | | | |
| erb-B2 | erb-b2 receptor | 179-183 | Colorectal cancer | CD28/4-1BB- | Third |
| | tyrosine kinase 2 | | | CD3ζ | |
| | (ERBB2) GenBank | | | | |
| | Accession Nos. | | | | |
| | NP_001005862.1; | | | | |
| | NP_001276865.1; | | | | |
| | NP_001276866.1; | | | | |
| | NP_001276867.1; | | | | |
| | NP_004439.2; | | | | |
| erb-B2 | | | Breast and others | ScFv-CD28- | Second |
| | | | | CD3ζ | |
| erb-B2 | | | Breast and others | ScFv-CD28- | Second |
| | | | | CD3ζ | |
| | | | | (Influenza) | |
| erb-B2 | | | Breast and others | ScFv- | Second |
| | | | | CD28mut- | |
| | | | | CD3ζ | |
| erb-B2 | | | Prostate cancer | ScFv-FcεRIγ | First |
| erb-B 2, 3, 4 | erb-b2 receptor | 179-183 | Breast and others | Heregulin- | Second |
| | tyrosine kinase 3 | | | CD3ζ | |
| | GenBank Accession | | | | |
| | Nos. | | | | |
| | NP_001005915.1; | | | | |
| | NP_001973.2; and | | | | |
| | erb-b2 receptor | | | | |
| | tyrosine kinase 2 | | | | |
| | GenBank Accession | | | | |
| | Nos. | | | | |
| | NP_001005862.1; | | | | |
| | NP_001276865.1; | | | | |
| | NP_001276866.1; | | | | |
| | NP_001276867.1; | | | | |
| | NP_004439.2 | | | | |
| erb-B 2, 3, 4 | | | Breast and others | ScFv-CD3ζ | First |
| FBP | far upstream element | 186-187; | Ovarian cancer | ScFv-FcεRIγ | First |
| | (FUSE) binding | | | | |
| | protein 1 (FUBP1) | | | | |
| | GenBank Accession | | | | |
| | No. | | | | |
| | NP_001290362.1; | | | | |
| | NP_003893.2; | | | | |
| FBP | | | Ovarian cancer | ScFv-FcεRIγ | First |
| | | | | (alloantigen) | |
| Fetal | | | Rhabdomyosarcoma | ScFv-CD3ζ | First |
| acetylcholine | | | | | |
| receptor | | | | | |
| GD2 | | | Neuroblastoma | ScFv-CD28 | First |
| GD2 | | | Neuroblastoma | ScFv-CD3ζ | First |
| GD2 | | | Neuroblastoma | ScFv-CD3ζ | First |
| GD2 | | | Neuroblastoma | ScFv-CD28- | Third |
| | | | | OX40-CD3ζ | |
| GD2 | | | Neuroblastoma | ScFv-CD3ζ | First |
| | | | | (VZV) | |
| GD3 | Graves disease, | | Melanoma | ScFv-CD3ξ | First |
| | susceptibility to, X- | | | | |
| | linked (GRDX); | | | | |
| | Gene ID 117189 | | | | |
| GD3 | | | Melanoma | ScFv-CD3ξ | First |
| Her2/neu | erb-b2 receptor | 179-183 | Medulloblastoma | ScFv-CD3ξ | First |
| | tyrosine kinase 2 | | | | |
| | GenBank Accession | | | | |
| | Nos. | | | | |
| | NP_001005862.1; | | | | |
| | NP_001276865.1; | | | | |
| | NP_001276866.1; | | | | |
| | NP_001276867.1; | | | | |
| | NP_004439.2 | | | | |
| Her2/neu | | | Lung malignancy | ScFv-CD28- | Second |
| | | | | CD3ζ | |
| Her2/neu | | | Advanced | ScFv-CD28- | Second |
| | | | osteosarcoma | CD3ζ | |
| Her2/neu | | | Glioblastoma | ScFv-CD28- | Second |
| | | | | CD3ζ | |
| IL-13R-a2 | interleukin 13 | 189 | Glioma | IL-13-CD28- | Third |
| | receptor, alpha 2 | | | 4-1BB-CD3ζ | |
| | (IL13RA2); | | | | |
| | GenBank Accession | | | | |
| | No. | | | | |
| | NP_000631.1 | | | | |
| IL-13R-a2 | | | Glioblastoma | IL-13-CD3ζ | Second |
| IL-13R-a2 | | | Medulloblastoma | IL-13-CD3ζ | Second |
| KDR | kinase insert domain | 190 | Tumor | ScFv-FcεRIγ | First |
| | receptor; GenBank | | neovasculature | | |
| | Accession No. | | | | |
| | NP_002244.1 | | | | |
| k-light chain | NFKBIA nuclear | 191 | B-cell malignancies | ScFv-CD3ζ | First |
| | factor of kappa light | | | | |
| | polypeptide gene | | | | |
| | enhancer in B-cells | | | | |
| | inhibitor, alpha; | | | | |
| | GenBank Accession | | | | |
| | No. NP_065390.1 | | | | |
| k-light chain | | | (B-NHL, CLL) | ScFv-CD28- | Second |
| | | | | CD3ζ vs | |
| | | | | CD3ζ | |
| LeY | | | Carcinomas | ScFv-FcεRIγ | First |
| LeY | | | Epithelial derived | ScFv-CD28- | Second |
| | | | tumors | CD3ζ | |
| L1 cell adhesion | L1 cell adhesion | 194-197 | Neuroblastoma | ScFv-CD3ζ | First |
| molecule | molecule (L1CAM), | | | | |
| | GenBank Accession | | | | |
| | Nos. NP_000416.1; | | | | |
| | NP_001137435.1; | | | | |
| | NP_001265045.1; | | | | |
| | NP_076493.1; | | | | |
| MAGE-A1 | Melanoma antigen | 133 | Melanoma | ScFV-CD4- | Second |
| | family A1 | | | FcεRIγ | |
| | (MAGEA1), | | | | |
| | GenBank Accession | | | | |
| | No. NP_004979.3 | | | | |
| MAGE-A1 | | | Melanoma | ScFV-CD28- | Second |
| | | | | FcεRIγ | |
| Mesothelin | MSLN, GenBank | 198-200 | Various tumors | ScFv-CD28- | Second |
| | Accession No. | | | CD3ζ | |
| | NP_001170826.1; | | | | |
| | NP_005814.2; | | | | |
| | NP_037536.2; | | | | |
| Mesothelin | | | Various tumors | ScFv-41BB- | Second |
| | | | | CD3ζ | |
| Mesothelin | | | Various tumors | ScFv-CD28- | Third |
| | | | | 41BB-CD3ζ | |
| Murine CMV | | | Murine CMV | Ly49H-CD3ζ | Second |
| infected cells | | | | | |
| MUC1 (target | NP_001018016.1; | 201; | Breast, Ovary | ScFV-CD28- | Third |
| antigens are from | NP_001018017.1; | 202; | | OX40-CD3ζ | |
| the extracellular | NP_001037855.1; | 105; | | | |
| domains of the | NP_001037856.1; | 204; | | | |
| MUC1 protein) | NP_001037857.1; | 106; | | | |
| | NP_001037858.1; | 206; | | | |
| | NP_001191214.1; | 107; | | | |
| | NP_001191215.1; | 208; | | | |
| | NP_001191216.1; | 108; | | | |
| | NP_001191217.1; | 210; | | | |
| | NP_001191218.1; | 109; | | | |
| | NP_001191219.1; | 212; | | | |
| | NP_001191220.1; | 110; | | | |
| | NP_001191221.1; | 214; | | | |
| | NP_001191222.1; | 111; | | | |
| | NP_001191223.1; | 216; | | | |
| | NP_001191224.1; | 112; | | | |
| | NP_001191225.1; | 218; | | | |
| | NP_001191226.1; | 113; | | | |
| | NP_002447.4 | 220; | | | |
| NKG2D ligands | Ligands of GenBank | 221 | Various tumors | NKG2D- | First |
| | Accession No. | | | CD3ζ | |
| | NP_031386.2 such | | | | |
| | as MHC class I | | | | |
| | chain-related A and | | | | |
| | B proteins and UL- | | | | |
| | 16 binding proteins | | | | |
| Oncofetal antigen | trophoblast | 222-223 | Various tumors | ScFV-CD3ζ | First |
| (h5T4) | glycoprotein | | | (vaccination) | |
| | (TPBG), GenBank | | | | |
| | Accession Nos. | | | | |
| | NP_001159864.1 | | | | |
| | and NP_006661.1 | | | | |
| PSCA | prostate specific | 98 | Prostate carcinoma | ScFv-b2c- | Second |
| | membrane antigen | | | CD3ζ | |
| | (PSCA), GenBank | | | | |
| | Accession No. | | | | |
| | NP_005663.2 | | | | |
| PSMA | folate hydrolase | 225-229 | Prostate/tumor | ScFv-CD3ζ | First |
| | (prostate-specific | | vasculature | | |
| | membrane antigen) 1 | | | | |
| | (FOLH1); GenBank | | | | |
| | Accession No. | | | | |
| | NP_001014986.1; | | | | |
| | NP_001180400.1; | | | | |
| | NP_001180401.1; | | | | |
| | NP_001180402.1; | | | | |
| | NP_004467.1 | | | | |
| PSMA | | | Prostate/tumor | ScFv-CD28- | Second |
| | | | vasculature | CD3ζ | |
| PSMA | | | Prostate/tumor | ScFv-CD3ζ | First |
| | | | vasculature | | |
| TAA targeted by | | | Various tumors | FceRI-CD28- | Third |
| mAb IgE | | | | CD3ζ (+ a- | |
| | | | | TAA IgE | |
| | | | | mAb) | |
| TAG-72 | | | Adenocarcinomas | scFv-CD3ζ | First |
| VEGF-R2 | Also known as | | Tumor | scFv-CD3ζ | First |
| | “KDR” | | neovasculature | | |

Table 15.

Tables 16-19 provide additional tumor antigens which can be used according to some embodiments of the invention.

TABLE 16
Tumor Antigens Resulting from Mutations

| | | | HLA | | | Lymphocyte |
|---|---|---|---|---|---|---|
| | | | Frequency^{b} | | SEQ ID | Stimulation |
| Gene/protein | Tumor | HLA^{a} | (%) | Peptide^{c} | NO: | Method |
| alpha-actinin-4 | lung | A2 | 44 | FIASNGVKLV | 475 | autologous |
| | carcinoma | | | | | tumor |
| | | | | | | cells |
| ARTC1 | melanoma | DR1 | 18 | YSVYFNLPADTIYTN^{h} | 476 | autologous |
| | | | | | | tumor |
| | | | | | | cells |
| -ABL BCR | chronic | A2 | 44 | SSKALQRPV | 477 | peptide |
| fusion protein | myeloid | B8 | 14 | GFKQSSKAL | 478 | peptide |
| (b3a2) | leukemia | DR4 | 24 | ATGFKQSSKALQRPV | 479 | peptide |
| | | | | AS | | |
| | | DR9 | 3 | ATGFKQSSKALQRPV | 480 | peptide |
| | | | | AS | | |
| B-RAF | melanoma | DR4 | 24 | EDLTVKIGDFGLATE | 481 | peptide |
| | | | | KSRWSGSHQFEQLS | | |
| CASP-5 | colorectal, | A2 | 44 | FLIIWQNTM^{g} | 482 | peptide |
| | gastric, and | | | | | |
| | endometrial | | | | | |
| | carcinoma | | | | | |
| CASP-8 | head and | B35 | 20 | FPSDSWCYF | 483 | autologous |
| | neck | | | | | tumor |
| | squamous | | | | | cell |
| | cells | | | | | |
| | carcinoma | | | | | |
| beta-catenin | melanoma | A24 | 20 | SYLDSGIHF | 484 | autologous |
| | | | | | | tumor |
| | | | | | | cells |
| Cdc27 | melanoma | DR4 | 24 | FSWAMDLDPKGA^{e} | 485 | autologous |
| | | | | | | tumor |
| | | | | | | cells |
| CDK4 | melanoma | A2 | 44 | ACDPHSGHFV | 486 | autologous |
| | | | | | | tumor |
| | | | | | | cells |
| CDKN2A | melanoma | A11 | 13 | AVCPWTWLR^{a} | 487 | autologous |
| | | | | | | tumor |
| | | | | | | cells |
| CLPP | melanoma | A2 | 44 | ILDKVLVHL | 488 | autologous |
| | | | | | | tumor |
| | | | | | | cells |
| COA-1 | colorectal | DR4 | 24 | TLYQDDTLTLQAAG^{e} | 489 | autologous |
| | carcinoma | | | | | tumor |
| | | | | | | cells |
| | | DR13 | 19 | TLYQDDTLTLQAAG^{e} | 490 | autologous |
| | | | | | | tumor |
| | | | | | | cells |
| -can fusion dek | myeloid | DR53 | 49 | TMKQICKKEIRRLHQY | 491 | peptide |
| protein | leukemia | | | | | |
| EFTUD2 | melanoma | A3 | 22 | KILDAVVAQK | 492 | autologous |
| | | | | | | tumor |
| | | | | | | cells |
| Elongation | lung | A68 | 8 | ETVSEQSNV | 493 | autologous |
| factor 2 | squamous | | | | | tumor |
| | CC | | | | | cells |
| -AML1 ETV6 | acute | A2 | 44 | RIAECILGM | 494 | peptide |
| fusion protein | lymphoblastic | DP5 | 3 | IGRIAECILGMNPSR | 495 | peptide |
| | leukemia | DP17 | 1 | IGRIAECILGMNPSR | 496 | peptide |
| FLT3-ITD | acute | A1 | 26 | YVDFREYEYY | 497 | peptide |
| | myelogenous | | | | | |
| | leukemia | | | | | |
| FN1 | melanoma | DR2 | 25 | MIFEKHGFRRTTPP | 498 | autologous |
| | | | | | | tumor |
| | | | | | | cells |
| GPNMB | melanoma | A3 | 22 | TLDWLLQTPK | 499 | autologous |
| | | | | | | tumor |
| | | | | | | cells |
| -LDLR | melanoma | DR1 | 18 | WRRAPAPGA | 500 | autologous |
| fucosyltransferase | | | | | | tumor |
| AS fusion | | | | | | cells |
| protein | | DR1 | 18 | PVTWRRAPA | 501 | autologous |
| | | | | | | tumor |
| | | | | | | cells |
| ^{d}HLA-A2 | renal cell | | | | 502 | autologous |
| | carcinoma | | | | | tumor |
| | | | | | | cells |
| ^{d}HLA-A11 | melanoma | | | | 503 | autologous |
| | | | | | | tumor |
| | | | | | | cells |
| hsp70-2 | renal cell | A2 | 44 | SLFEGIDIYT | 504 | autologous |
| | carcinoma | | | | | tumor |
| | | | | | | cells |
| | bladder | B44 | 21 | AEPINIQTW | 505 | autologous |
| | tumor | | | | | tumor |
| | | | | | | cells |
| MART2 | melanoma | A1 | 26 | FLEGNEVGKTY | 506 | autologous |
| | | | | | | tumor |
| | | | | | | cells |
| ME1 | non-small | A2 | 44 | FLDEFMEGV | 507 | autologous |
| | cell lung | | | | | tumor |
| | carcinoma | | | | | cells |
| ^{f}MUM-1 | melanoma | B44 | 21 | EEKLIVVLF | 508 | autologous |
| | | | | | | tumor |
| | | | | | | cells |
| MUM-2 | melanoma | B44 | 21 | SELFRSGLDSY | 509 | autologous |
| | | | | | | tumor |
| | | | | | | cells |
| | | Cw6 | 18 | FRSGLDSYV | 510 | autologous |
| | | | | | | tumor |
| | | | | | | cells |
| MUM-3 | melanoma | A68 | 8 | EAFIQPITR | 511 | autologous |
| | | | | | | tumor |
| | | | | | | cells |
| neo-PAP | melanoma | DR7 | 25 | RVIKNSIRLTL^{e} | 512 | autologous |
| | | | | | | tumor |
| | | | | | | cells |
| Myosin class I | melanoma | A3 | 22 | KINKNPKYK | 513 | expansion |
| | | | | | | of TIL |
| | | | | | | with IL-2 |
| NFYC | lung | B52 | 5 | QQITKTEV | 514 | autologous |
| | squamous | | | | | tumor |
| | cell | | | | | cells |
| | carcinoma | | | | | |
| OGT | colorectal | A2 | 44 | SLYKFSPFPL^{g} | 515 | peptide |
| | carcinoma | | | | | |
| OS-9 | melanoma | B44 | 21 | KELEGILLL | 516 | autologous |
| | | | | | | tumor |
| | | | | | | cells |
| p53 | head and | A2 | 44 | VVPCEPPEV | 517 | peptide |
| | neck | | | | | |
| | squamous | | | | | |
| | cell | | | | | |
| | carcinoma | | | | | |
| -RARalphapml | promyelocytic | DR11 | 25 | NSNHVASGAGEAAIE | 518 | peptide |
| fusion protein | leukemia | | | TQSSSSEEIV | | |
| PRDX5 | melanoma | A2 | 44 | LLLDDLLVSI | 519 | peptide |
| PTPRK | melanoma | DR10 | 3 | PYYFAAELPPRNLPEP | 520 | autologous |
| | | | | | | tumor |
| | | | | | | cells |
| K-ras | pancreatic | B35 | 20 | VVVGAVGVG | 521 | peptide |
| | adenocarcinoma | | | | | |
| N-ras | melanoma | A1 | 26 | ILDTAGREEY | 522 | autologous |
| | | | | | | tumor |
| | | | | | | cells |
| RBAF600 | melanoma | B7 | 17 | RPHVPESAF | 523 | autologous |
| | | | | | | tumor |
| | | | | | | cells |
| SIRT2 | melanoma | A3 | 22 | KIFSEVTLK | 524 | autologous |
| | | | | | | tumor |
| | | | | | | cells |
| SNRPD1 | melanoma | B38 | 5 | SHETVIIEL | 525 | autologous |
| | | | | | | tumor |
| | | | | | | cells |
| -SSX1 or -SYT | sarcoma | B7 | 17 | QRPYGYDQIM | 526 | peptide |
| SSX2 fusion | | | | | | |
| protein | | | | | | |
| TGF-betaRII | colorectal | A2 | 44 | RLSSCVPVA^{g} | 527 | peptide |
| | carcinoma | | | | | |
| Triosephosphate | melanoma | DR1 | 18 | GELIGILNAAKVPAD | 528 | autologous |
| isomerase | | | | | | tumor |
| | | | | | | cells |

Table 6:.
^{b}Frequency in Caucasians, based on (1) Marsh SGE, Parham P, Barber LD. The HLA Factsbook (Academic Press, 2000) for HLA-A, B, C, and DR, and (2) Colombani J. HLA, Fonctions immunitaires et applications medicales (John Libbey Eurotext, 1993) for HLA-DP and DR.
^{c}The residues modified by the mutation are indicated in red.
^{d}The mutation affects the HLA gene itself.
^{e}The mutation is not located in the region encoding the peptide.
^{f}Link to GenBank.
^{g}Frameshift product.
^{h}The mutation creates a start codon (ATG) that opens an alternative ORF encoding the antigenic peptide. This peptide is recognized by regulatory T cells (Tregs).
ⁱIt was reported that this peptide is not naturally processed (Popovic 2011).

TABLE 17
Shared Tumor-Specific Antigens

| | | HLA | | SEQ | Lymphocyte |
|---|---|---|---|---|---|
| | | Frequency^{b} | | ID | Stimulation |
| Gene | HLA | (%) | Peptide | NO: | Method |
| BAGE-1 | Cw16 | 7 | AARAVFLAL | 529 | autologous |
| | | | | | tumor cells |
| Cyclin-A1 | A2 | 44 | FLDRFLSCM | 530 | peptide |
| | A2 | 44 | SLIAAAAFCLA | 531 | peptide |
| GAGE- | Cw6 | 18 | YRPRPRRY | 532 | autologous |
| 1.2.8 | | | | | tumor cells |
| GAGE- | A29 | 6 | YYWPRPRRY | 533 | autologous |
| 3.4.5.6.7 | | | | | tumor cells |
| ^{f}GnTV | A2 | 44 | VLPDVFIRC(V) | 534 | autologous |
| | | | | | tumor cells |
| HERV-K- | A2 | 44 | MLAVISCAV | 535 | autologous |
| MEL | | | | | tumor cells |
| KK-LC-1 | B15 | 13 | RQKRILVNL | 536 | autologous |
| | | | | | tumor cells |
| KM-HN-1 | A24 | 20 | NYNNFYRFL | 537 | peptide |
| | A24 | 20 | EYSKECLKEF | 538 | peptide |
| | A24 | 20 | EYLSLSDKI | 539 | peptide |
| LAGE-1 | A2 | 44 | MLMAQEALAFL | 540 | autologous |
| | | | | | tumor cells |
| | A2 | 44 | SLLMWITQC | 541 | peptide |
| | A31 | 5 | LAAQERRVPR | 542 | autologous |
| | | | | | tumor cells |
| | A68 | 8 | ELVRRILSR | 543 | adenovirus- |
| | | | | | dendritic |
| | | | | | cells |
| | B7 | 17 | APRGVRMAV | 544 | adenovirus- |
| | | | | | APC |
| | DP4 | 75 | SLLMWITQCFLPVF | 545 | peptide |
| | DR3 | 21 | QGAMLAAQERRVPRAAEVPR | 546 | protein |
| | DR4 | 24 | AADHRQLQLSISSCLQQL | 547 | protein |
| | DR11 | 25 | CLSRRPWKRSWSAGSCPGMP | 548 | peptide |
| | | | HL | | |
| | DR12 | 5 | CLSRRPWKRSWSAGSCPGMP | 549 | peptide |
| | | | HL | | |
| | DR13 | 19 | ILSRDAAPLPRPG | 550 | autologous |
| | | | | | tumor cells |
| | DR15 | 20 | AGATGGRGPRGAGA | 551 | protein |
| MAGE-A1 | A1 | 26 | EADPTGHSY | 552 | autologous |
| | | | | | tumor cells |
| | A2 | 44 | KVLEYVIKV | 553 | peptide |
| | A3 | 22 | SLFRAVITK | 554 | poxvirus- |
| | | | | | dendritic |
| | | | | | cells^{c} |
| | A68 | 8 | EVYDGREHSA | 555 | poxvirus- |
| | | | | | dendritic |
| | | | | | cells |
| | B7 | 17 | RVRFFFPSL | 556 | poxvirus- |
| | | | | | dendritic |
| | | | | | cells |
| | B35 | 20 | EADPTGHSY | 557 | poxvirus- |
| | | | | | dendritic |
| | | | | | cells |
| | B37 | 3 | REPVTKAEML | 558 | autologous |
| | | | | | tumor cells |
| | B44 | 21 | KEADPTGHSY | 559 | poxvirus- |
| | | | | | dendritic |
| | | | | | cells |
| | B53 | 2 | DPARYEFLW | 560 | poxvirus- |
| | | | | | dendritic |
| | | | | | cells |
| | B57 | 8 | ITKKVADLVGF | 561 | ALVAC- |
| | | | | | dendritic |
| | | | | | cells |
| | Cw2 | 10 | SAFPTTINF | 562 | poxvirus- |
| | | | | | dendritic |
| | | | | | cells |
| | Cw3 | 17 | SAYGEPRKL | 563 | poxvirus- |
| | | | | | dendritic |
| | | | | | cells |
| | Cw7 | 41 | RVRFFFPSL | 564 | peptide |
| | Cw16 | 7 | SAYGEPRKL | 565 | autologous |
| | | | | | tumor cells |
| | DP4 | 75 | TSCILESLFRAVITK | 566 | peptide |
| | DP4 | 75 | PRALAETSYVKVLEY | 567 | peptide |
| | DR13 | 19 | FLLLKYRAREPVTKAE | 568 | protein |
| | DR15 | 20 | EYVIKVSARVRF | 569 | protein |
| MAGE-A2 | A2 | 44 | YLQLVFGIEV | 570 | peptide |
| | A24 | 20 | EYLQLVFGI | 571 | peptide |
| | B37 | 3 | REPVTKAEML | 572 | autologous |
| | | | | | tumor cells |
| | Cw7 | 41 | EGDCAPEEK | 573 | lentivirus- |
| | | | | | dendritic |
| | | | | | cells |
| | DR13 | 19 | LLKYRAREPVTKAE | 574 | protein |
| MAGE-A3 | A1 | 26 | EVDPIGHLY | 575 | autologous |
| | | | | | tumor cells |
| | A2 | 44 | FLWGPRALV^{d} | 576 | peptide |
| | A2 | 44 | KVAELVHFL | 577 | peptide |
| | A24 | 20 | TFPDLESEF | 578 | peptide |
| | A24 | 20 | VAELVHFLL | 579 | peptide |
| | B18 | 6 | MEVDPIGHLY | 580 | adeno- |
| | | | | | dendritic |
| | | | | | cells |
| | B35 | 20 | EVDPIGHLY | 581 | poxvirus- |
| | | | | | dendritic |
| | | | | | cells |
| | B37 | 3 | REPVTKAEML | 582 | autologous |
| | | | | | tumor cells |
| | B40 | 6 | AELVHFLLLⁱ | 583 | adeno- |
| | | | | | dendritic |
| | | | | | cells |
| | B44 | 21 | MEVDPIGHLY | 584 | peptide |
| | B52 | 5 | WQYFFPVIF | 585 | retrovirus- |
| | | | | | dendritic |
| | | | | | cells^{h} |
| | Cw7 | 41 | EGDCAPEEK | 586 | lentivirus- |
| | | | | | dendritic |
| | | | | | cells |
| | DP4 | 75 | KKLLTQHFVQENYLEY | 587 | protein |
| | DP4 | 75 | RKVAELVHFLLLKYR | 588 | peptide |
| | DQ6 | 63 | KKLLTQHFVQENYLEY | 589 | peptide |
| | DR1 | 18 | ACYEFLWGPRALVETS | 590 | protein |
| | DR4 | 24 | RKVAELVHFLLLKYR | 591 | peptide |
| | DR4 | 24 | VIFSKASSSLQL | 592 | peptide |
| | DR7 | 25 | VIFSKASSSLQL | 593 | peptide |
| | DR7 | 25 | VFGIELMEVDPIGHL | 594 | peptide |
| | DR11 | 25 | GDNQIMPKAGLLIIV | 595 | peptide |
| | DR11 | 25 | TSYVKVLHHMVKISG | 596 | protein |
| | DR13 | 19 | RKVAELVHFLLLKYRA | 597 | protein |
| | DR13 | 19 | FLLLKYRAREPVTKAE | 598 | protein |
| MAGE-A4 | A1 | 26 | EVDPASNTY^{j} | 599 | peptide after |
| | | | | | tetramer |
| | | | | | sorting |
| | 42 | 44 | GVYDGREHTV | 600 | adeno- |
| | | | | | dendritic |
| | | | | | cells |
| | A24 | 20 | NYKRCFPVI | 601 | peptide |
| | B37 | 3 | SESLKMIF | 602 | poxvirus- |
| | | | | | dendritic |
| | | | | | cells |
| MAGE-A6 | A34 | 1 | MVKISGGPR | 603 | autologous |
| | | | | | tumor cells |
| | B35 | 20 | EVDPIGHVY | 604 | autologous |
| | | | | | tumor cells |
| | B37 | 3 | REPVTKAEML | 605 | autologous |
| | | | | | tumor cells |
| | Cw7 | 41 | EGDCAPEEK | 606 | lentivirus- |
| | | | | | dendritic |
| | | | | | cells |
| | Cw16 | 7 | ISGGPRISY | 607 | autologous |
| | | | | | tumor cells |
| | DR13 | 19 | LLKYRAREPVTKAE | 608 | protein |
| MAGE-A9 | A2 | 44 | ALSVMGVYV | 609 | peptide |
| MAGE- | A2 | 44 | GLYDGMEHL^{l} | 610 | autologous |
| A10 | | | | | tumor cells |
| | B53 | 2 | DPARYEFLW | 611 | poxvirus- |
| | | | | | dendritic |
| | | | | | cells |
| MAGE- | A2^{g} | 44 | FLWGPRALV^{e} | 612 | peptide |
| A12^{m} | Cw7 | 41 | VRIGHLYIL | 613 | autologous |
| | | | | | tumor cells |
| | Cw7 | 41 | EGDCAPEEK | 614 | lentivirus- |
| | | | | | dendritic |
| | | | | | cells |
| | DP4 | 75 | REPFTKAEMLGSVIR | 615 | peptide |
| | DR13 | 19 | AELVHFLLLKYRAR | 616 | protein |
| MAGE-C1 | A2 | 44 | ILFGISLREV | 617 | peptide |
| | A2 | 44 | KVVEFLAML | 529 | peptide |
| | DQ6 | 63 | SSALLSIFQSSPE | 530 | peptide |
| | DQ6 | 63 | SFSYTLLSL | 531 | peptide |
| | DR15 | 20 | VSSFFSYTL | 532 | peptide |
| MAGE-C2 | A2 | 44 | LLFGLALIEV | 533 | autologous |
| | | | | | tumor cells |
| | A2 | 44 | ALKDVEERV | 534 | autologous |
| | | | | | tumor cells |
| | B44 | 21 | SESIKKKVL | 535 | autologous |
| | | | | | tumor cells |
| | B57 | 8 | ASSTLYLVF | 536 | autologous |
| | | | | | tumor cells |
| | DR15 | 20 | SSTLYLVFSPSSFST | 537 | peptide |
| ^{k}mucin | | | PDTRPAPGSTAPPAHGVTSA | 538 | transfected B |
| | | | | | cells |
| NA88-A | B13 | 6 | QGQHFLQKV | 539 | tumor- |
| | | | | | infiltrating |
| | | | | | lymphocytes |
| NY-ESO-1/ | A2 | 44 | SLLMWITQC | 540 | autologous |
| LAGE-2 | | | | | tumor cells |
| | A2 | 44 | MLMAQEALAFL | 541 | autologous |
| | | | | | tumor cells |
| | A24 | 20 | YLAMPFATPME | 542 | peptide |
| | A31 | 5 | ASGPGGGAPR | 543 | autologous |
| | | | | | tumor cells |
| | A31 | 5 | LAAQERRVPR | 544 | autologous |
| | | | | | tumor cells |
| | A68 | 8 | TVSGNILTIR | 545 | mRNA- |
| | | | | | transfected |
| | | | | | cells |
| | B7 | 17 | APRGPHGGAASGL | 546 | peptide |
| | B35 | 20 | MPFATPMEA | 547 | autologous |
| | | | | | tumor cells |
| | B49 | | KEFTVSGNILTI | 548 | mRNA- |
| | | | | | transfected |
| | | | | | cells |
| | B51 | 12 | MPFATPMEA | 549 | adenovirus- |
| | | | | | APC |
| | B52 | 5 | FATPMEAEL | 550 | peptide |
| | C12 | 12 | FATPMEAELAR | 551 | peptide |
| | Cw3 | 17 | LAMPFATPM | 552 | adenovirus- |
| | | | | | PBMC |
| | Cw6 | 18 | ARGPESRLL | 553 | adenovirus- |
| | | | | | PBMC^{d} |
| | DP4 | 75 | SLLMWITQCFLPVF | 554 | peptide |
| | DP4 | 75 | LLEFYLAMPFATPMEAELAR | 555 | peptide |
| | | | RSLAQ | | |
| | DR1 | 18 | LLEFYLAMPFATPMEAELAR | 556 | peptide |
| | | | RSLAQ | | |
| | DR1 | 18 | EFYLAMPFATPM | 557 | protein |
| | DR1 | 18 | PGVLLKEFTVSGNILTIRLTA | 558 | peptide |
| | | | ADHR | | |
| | DR2 | 25 | RLLEFYLAMPFA | 559 | protein |
| | DR3 | 21 | QGAMLAAQERRVPRAAEVPR | 560 | protein |
| | DR4 | 24 | PFATPMEAELARR | 561 | peptide |
| | DR4 | 24 | PGVLLKEFTVSGNILTIRLT | 562 | peptide and |
| | | | | | protein |
| | DR4 | 24 | VLLKEFTVSG | 563 | peptide |
| | DR4 | 24 | AADHRQLQLSISSCLQQL | 564 | protein |
| | DR4 | 24 | LLEFYLAMPFATPMEAELAR | 565 | peptide |
| | | | RSLAQ | | |
| | DR52b | 25 | LKEFTVSGNILTIRL | 566 | protein |
| | DR7 | 25 | PGVLLKEFTVSGNILTIRLTA | 567 | peptide |
| | | | ADHR | | |
| | DR7 | 25 | LLEFYLAMPFATPMEAELAR | 568 | peptide |
| | | | RSLAQ | | |
| | DR8 | 4 | KEFTVSGNILT | 569 | peptide |
| | DR9 | 3 | LLEFYLAMPFATPM | 570 | peptide |
| | DR15 | 20 | AGATGGRGPRGAGA | 571 | protein |
| SAGE | A24 | 20 | LYATVIHDI | 572 | peptide |
| Sp17 | A1 | 26 | ILDSSEEDK | 573 | protein |
| SSX-2 | A2 | 44 | KASEKIFYV | 574 | autologous |
| | | | | | tumor cells |
| | DP1 | 14 | EKIQKAFDDIAKYFSK | 575 | peptide |
| | DR1 | 18 | FGRLQGISPKI | 576 | peptide |
| | DR3 | 21 | WEKMKASEKIFYVYMKRK | 577 | peptide |
| | DR4 | 24 | KIFYVYMKRKYEAMT | 578 | peptide] |
| | DR11 | 25 | KIFYVYMKRKYEAM | 579 | protein |
| SSX-4 | DP10 | 2 | INKTSGPKRGKHAWTHRLRE | 580 | peptide |
| | DR3 | 21 | YFSKKEWEKMKSSEKIVYVY | 581 | peptide |
| | DR8 | 4 | MKLNYEVMTKLGFKVTLPPF | 582 | peptide |
| | DR8 | 4 | KHAWTHRLRERKQLVVYEEI | 583 | peptide |
| | DR11 | 25 | LGFKVTLPPFMRSKRAADFH | 584 | peptide |
| | DR15 | 20 | KSSEKIVYVYMKLNYEVMTK | 585 | peptide |
| | DR52 | 41 | KHAWTHRLRERKQLVVYEEI | 586 | peptide |
| TAG-1 | A2 | 44 | SLGWLFLLL | 587 | peptide |
| | B8 | 14 | LSRLSNRLL | 588 | peptide |
| TAG-2 | B8 | 14 | LSRLSNRLL | 589 | peptide |
| TRAG-3 | DR1 | 18 | CEFHACWPAFTVLGE | 590 | peptide |
| | DR4 | 24 | CEFHACWPAFTVLGE | 591 | peptide |
| | DR7 | 25 | CEFHACWPAFTVLGE | 592 | peptide |
| -TRP2 | A68 | 8 | EVISCKLIKR | 593 | autologous |
| INT2^{g} | | | | | tumor cells |
| XAGE- | A2 | 44 | RQKKIRIQL | 594 | peptide |
| 1b/GAGED | DR4 | 24 | HLGSRQKKIRIQLRSQ | 595 | peptide |
| 2a | DR9 | 3 | CATWKVICKSCISQTPG | 596 | autologous |
| | | | | | tumor cells |

^{b}Frequency in Caucasians, based on (1) Marsh SGE, Parham P, Barber LD. The HLA Factsbook (Academic Press, 2000) for HLA-A, B, C, and DR, and (2) Colombani J. HLA, Fonctions immunitaires et applications médicales (John Libbey Eurotext, 1993) for HLA-DP and DR.
^{c}ALVAC recombinant poxvirus carrying the entire gene were used to infect dendritic cells.
^{d}Only processed by the intermediate proteasome β5i (Guillaume, 2010).
^{e}Same peptide as MAGE-A3/A2 (aa 271-279).
^{f}Aberrant transcript of N-acetyl glucosaminyl transferase V (GnTV) that is found only in melanomas.
^{g}Incompletely spliced transcript found only in melanomas.
^{h}Retrovirus carrying the entire gene were used to infect dendritic cells.
ⁱThe processing of this peptide requires the immunoproteasome.
^{j}This peptide is encoded by allele MAGE-4a, which is expressed in one third of MAGE-4 positive tumor samples. The other allele, namely MAGE-4b, encodes peptide EVDPTSNTY.
^{k}MHC-unrestricted recognition by CTL of a repeated motif that is unmasked in tumors due to mucin underglycosylation. Mucin underglycosylation also occurs in breast duct epithelial cells during lactation, but only at the extracellular apical surface, which is not accessible to T cells.
^{l}Only processed by the intermediate proteasome β1iβ51 (Guillaume, 2010).
^{m}Not only expressed in testis but also in brain.

TABLE 18
Differentiation Antigens

| | | | HLA | | | Lymphocyte |
|---|---|---|---|---|---|---|
| Gene/ | | | Frequency^{b} | | SEQ ID | Stimulation |
| protein | Tumor | HLA^{a} | (%) | Peptide | NO: | Method |
| CEA | gut | A2 | 44 | YLSGANLNL^{g} | 597 | peptide |
| | carcinoma | A2 | 44 | IMIGVLVGV | 598 | peptide |
| | | A2 | 44 | GVLVGVALI | 599 | peptide |
| | | A3 | 22 | HLFGYSWYK | 600 | peptide |
| | | A24 | 20 | QYSWFVNGTF | 601 | peptide |
| | | A24 | 20 | TYACFVSNL | 602 | peptide |
| | | DR3 | 21 | AYVCGIQNSVSANRS | 603 | peptide |
| | | DR4 | 24 | DTGFYTLHVIKSDLVNEE | 604 | peptide |
| | | | | ATGQFRV | | |
| | | DR4 | 24 | YSWRINGIPQQHTQV | 605 | peptide |
| | | DR7 | 25 | TYYRPGVNLSLSC | 606 | peptide |
| | | DR7 | 25 | EIIYPNASLLIQN | 607 | peptide |
| | | DR9 | 3 | YACFVSNLATGRNNS | 608 | peptide |
| | | DR11 | 25 | LWWVNNQSLPVSP | 609 | peptide |
| | | DR13 | 19 | LWWVNNQSLPVSP | 610 | peptide |
| | | DR14 | 6 | LWWVNNQSLPVSP | 611 | peptide |
| | | DR14 | 6 | EIIYPNASLLIQN | 612 | peptide |
| | | DR14 | 6 | NSIVKSITVSASG | 613 | peptide |
| gp100/ | melanoma | A2 | 44 | KTWGQYWQV | 614 | autologous |
| Pmel17 | | | | | | tumor |
| | | | | | | cells |
| | | A2 | 44 | (A)MLGTHTMEV | 615 | peptide |
| | | A2 | 44 | ITDQVPFSV | 616 | autologous |
| | | | | | | tumor |
| | | | | | | cells |
| | | A2 | 44 | YLEPGPVTA | 617 | autologous |
| | | | | | | tumor |
| | | | | | | cells |
| | | A2 | 44 | LLDGTATLRL | 618 | autologous |
| | | | | | | tumor |
| | | | | | | cells |
| | | A2 | 44 | VLYRYGSFSV | 619 | autologous |
| | | | | | | tumor |
| | | | | | | cells |
| | | A2 | 44 | SLADTNSLAV | 620 | peptide |
| | | A2 | 44 | RLMKQDFSV | 621 | autologous |
| | | | | | | tumor |
| | | | | | | cells |
| | | A2 | 44 | RLPRIFCSC | 622 | autologous |
| | | | | | | tumor |
| | | | | | | cells |
| | | A3 | 22 | LIYRRRLMK | 623 | autologous |
| | | | | | | tumor |
| | | | | | | cells |
| | | A3 | 22 | ALLAVGATK | 624 | autologous |
| | | | | | | tumor |
| | | | | | | cells |
| | | A3 | 22 | IALNFPGSQK | 625 | peptide |
| | | A3 | 22 | RSYVPLAHR | 626 | autologous |
| | | | | | | tumor |
| | | | | | | cells |
| | | A3 | 22 | ALNFPGSQK | 627 | peptide |
| | | A11 | 13 | ALNFPGSQK | 628 | peptide |
| | | A24 | 20 | VYFFLPDHL | 629 | autologous |
| | | | | | | tumor |
| | | | | | | cells |
| | | A32 | 8 | RTKQLYPEW | 630 | autologous |
| | | | | | | tumor |
| | | | | | | cells |
| | | A68 | 8 | HTMEVTVYHR | 631 | autologous |
| | | | | | | tumor |
| | | | | | | cells |
| | | B7 | 17 | SSPGCQPPA | 632 | autologous |
| | | | | | | tumor |
| | | | | | | cells |
| | | B35 | 20 | VPLDCVLYRY | 633 | autologous |
| | | | | | | tumor |
| | | | | | | cells |
| | | B35 | 20 | LPHSSSHWL | 634 | autologous |
| | | | | | | tumor |
| | | | | | | cells |
| | | Cw8 | —^{c} | SNDGPTLI | 635 | autologous |
| | | | | | | tumor |
| | | | | | | cells |
| | | DQ6 | 63 | GRAMLGTHTMEVTVY | 636 | peptide |
| | | DR4 | 24 | WNRQLYPEWTEAQRLD | 637 | peptide |
| | | DR7 | 25 | TTEWVETTARELPIPEPE | 638 | protein |
| | | DR7 | 25 | TGRAMLGTHTMEVTVYH | 639 | retrovirus - |
| | | | | | | dendritic |
| | | | | | | cells |
| | | DR53 | 49 | GRAMLGTHTMEVTVY | 640 | peptide |
| mammaglobin-A | breast | A3 | 22 | PLLENVISK | 641 | peptide |
| | cancer | | | | | |
| Melan-A/ | melanoma | A2 | 44 | (E)AAGIGILTV | 642 | autologous |
| MART-1 | | | | | | tumor |
| | | | | | | cells |
| | | A2 | 44 | ILTVILGVL | 643 | autologous |
| | | | | | | tumor |
| | | | | | | cells |
| | | B35 | 20 | EAAGIGILTV | 644 | autologous |
| | | | | | | tumor |
| | | | | | | cells |
| | | B45 | 2 | AEEAAGIGIL(T) | 645 | autologous |
| | | | | | | tumor |
| | | | | | | cells |
| | | Cw7 | 41 | RNGYRALMDKS | 646 | peptide |
| | | DP5 | 3 | YTTAEEAAGIGILTVILGV | 647 | peptide |
| | | | | LLLIGCWYCRR | | |
| | | DQ6 | 63 | EEAAGIGILTVI | 648 | peptide |
| | | DR1 | 18 | AAGIGILTVILGVL | 649 | peptide |
| | | DR1 | 18 | APPAYEKLpSAEQ^{f} | 650 | peptide |
| | | DR3 | 21 | EEAAGIGILTVI | 651 | peptide |
| | | DR4 | 24 | RNGYRALMDKSLHVGTQ | 652 | peptide |
| | | | | CALTRR | | |
| | | DR11 | 25 | MPREDAHFIYGYPKKGHG | 653 | peptide |
| | | | | HS | | |
| | | DR52 | 41 | KNCEPVVPNAPPAYEKLS | 654 | peptide |
| | | | | AE | | |
| NY-BR-1 | breast | A2 | 44 | SLSKILDTV | 655 | peptide |
| | cancer | | | | | |
| OA1 | melanoma | A24 | 20 | LYSACFWWL | 656 | peptide |
| PAP | prostate | A2 | 44 | FLFLLFFWL | 657 | peptide |
| | cancer | A2 | 44 | TLMSAMTNL | 658 | peptide |
| | | A2 | 44 | ALDVYNGLL | 659 | peptide |
| PSA | prostate | A2 | 44 | FLTPKKLQCV | 660 | peptide |
| | carcinoma | A2 | 44 | VISNDVCAQV | 661 | peptide |
| RAB38/ | melanoma | A2 | 44 | VLHWDPETV | 662 | peptide |
| NY- | | | | | | |
| MEL-1 | | | | | | |
| TRP-1/ | melanoma | A31 | 5 | MSLQRQFLR | 663 | autologous |
| gp75 | | | | | | tumor |
| | | | | | | cells |
| | | DR4 | 24 | ISPNSVFSQWRVVCDSLED | 664 | peptide |
| | | | | YD | | |
| | | DR15 | 20 | SLPYWNFATG | 665 | autologous |
| | | | | | | tumor |
| | | | | | | cells |
| | | DR17 | 21 | SQWRVVCDSLEDYDT | 666 | peptide |
| TRP-2 | melanoma | A2 | 44 | SVYDFFVWL | 667 | peptide |
| | | A2 | 44 | TLDSQVMSL | 668 | peptide |
| | | A31 | 5 | LLGPGRPYR | 669 | autologous |
| | | | | | | tumor |
| | | | | | | cells |
| | | A33 | 5 | LLGPGRPYR | 670 | autologous |
| | | | | | | tumor |
| | | | | | | cells |
| | | Cw8 | —^{c} | ANDPIFVVL | 671 | autologous |
| | | | | | | tumor |
| | | | | | | cells |
| | | DR3 | 21 | QCTEVRADTRPWSGP | 672 | peptide |
| | | DR15 | 20 | ALPYWNFATG | 673 | autologous |
| | | | | | | tumor |
| | | | | | | cells |
| tyrosinase | melanoma | A1 | 26 | KCDICTDEY | 674 | autologous |
| | | | | | | tumor |
| | | | | | | cells |
| | | A1 | 26 | SSDYVIPIGTY | 675 | autologous |
| | | | | | | tumor |
| | | | | | | cells |
| | | A2 | 44 | MLLAVLYCL | 676 | autologous |
| | | | | | | tumor |
| | | | | | | cells |
| | | A2 | 44 | CLLWSFQTSA | 677 | peptide |
| | | A2 | 44 | YMDGTMSQV | 678 | autologous |
| | | | | | | tumor |
| | | | | | | cells |
| | | A24 | 20 | AFLPWHRLF | 679 | autologous |
| | | | | | | tumor |
| | | | | | | cells |
| | | A24 | 20 | IYMDGTADFSF | 680 | autologous |
| | | | | | | tumor |
| | | | | | | cells |
| | | A26 | 8 | QCSGNFMGF | 681 | autologous |
| | | | | | | tumor |
| | | | | | | cells |
| | | B35 | 20 | TPRLPSSADVEF | 682 | autologous |
| | | | | | | tumor |
| | | | | | | cells |
| | | B35 | 20 | LPSSADVEF | 683 | autologous |
| | | | | | | tumor |
| | | | | | | cells |
| | | B38 | 5 | LHHAFVDSIF | 684 | autologous |
| | | | | | | tumor |
| | | | | | | cells |
| | | B44 | 21 | SEIWRDIDF^{d} | 685 | autologous |
| | | | | | | tumor |
| | | | | | | cells |
| | | DR4 | 24 | QNILLSNAPLGPQFP | 686 | autologous |
| | | | | | | tumor |
| | | | | | | cells |
| | | DR4 | 24 | SYLQDSDPDSFQD | 687 | autologous |
| | | | | | | tumor |
| | | | | | | cells |
| | | DR15 | 20 | FLLHHAFVDSIFEQWLQR | 688 | autologous |
| | | | | HRP | | tumor |
| | | | | | | cells |

^{b}Frequency in Caucasians, based on (1) Marsh SGE, Parham P, Barber LD. The HLA Factsbook (Academic Press, 2000) for HLA-A, B, C, and DR, and (2) Colombani J. HLA, Fonctions immunitaires et applications médicales (John Libbey Eurotext, 1993) for HLA-DP and DR.
^{c}Not available.
^{d}Different alleles encoding tyrosinase have been described. In 50% of Caucasians, the serine residue of nonapeptide SEIWRDIDF is replaced by a tyrosine.
^{e}The peptide is composed of two non-contiguous fragments that are spliced by the proteasome.
^{f}Phosphopeptide.
^{g}Seems to be poorly processed by tumor cells (Fauquembergue, 2010).

TABLE 19
Antigens Overexpressed in Tumors

| | Normal | | HLA | | SEQ | Lymphocyte |
|---|---|---|---|---|---|---|
| | tissue | | Frequency^{b} | | ID | Stimulation |
| Gene | expression | HLA^{a} | (%) | Peptide | NO: | Method |
| adipophilin | adipocytes, | A2 | 44 | SVASTITGV | 689 | peptide |
| | macrophages | | | | | |
| AIM-2 | ubiquitous | A1 | 26 | RSDSGQQARY | 690 | autologous |
| | (low level) | | | | | tumor cells |
| ALDH1A1 | mucosa, | A2 | 44 | LLYKLADLI | 691 | peptide |
| | keratinocytes | | | | | |
| BCLX (L) | ubiquitous | A2 | 44 | YLNDHLEPWI | 692 | peptide |
| | (low level) | | | | | |
| BING-4 | ubiquitous | A2 | 44 | CQWGRLWQL | 693 | anti-CD3 |
| | (low level) | | | | | |
| CALCA | thyroid | A2 | 44 | VLLQAGSLHA | 694 | autologous |
| | | | | | | tumor cells |
| CD45 | proliferating | A24 | 20 | KFLDALISL | 695 | peptide |
| | cells, | | | | | |
| | testis, | | | | | |
| | multiple | | | | | |
| | tissues | | | | | |
| | (low level) | | | | | |
| CD274 | multiple | A2 | 44 | LLNAFTVTV | 696 | peptide |
| | tissues | | | | | |
| | (lung, | | | | | |
| | heart, | | | | | |
| | dendritic | | | | | |
| | cells, ...) | | | | | |
| | and | | | | | |
| | induced | | | | | |
| | by IFN-γ | | | | | |
| CPSF | ubiquitous | A2 | 44 | KVHPVIWSL | 697 | autologous |
| | (low level) | | | | | tumor cells |
| | | A2 | 44 | LMLQNALTTM | 698 | autologous |
| | | | | | | tumor cells |
| cyclin D1 | ubiquitous | A2 | 44 | LLGATCMFV | 699 | peptide |
| | (low level) | DR4 | 24 | NPPSMVAAGSVV | 700 | peptide |
| | | | | AAV | | |
| DKK1 | testis, | A2 | 44 | ALGGHPLLGV | 701 | peptide |
| | prostate, | | | | | |
| | mesenchymal | | | | | |
| | stem | | | | | |
| | cells | | | | | |
| ENAH (hMena) | breast, | A2 | 44 | TMNGSKSPV | 702 | peptide |
| | prostate | | | | | |
| | stroma | | | | | |
| | and | | | | | |
| | epithelium | | | | | |
| | of colon- | | | | | |
| | rectum, | | | | | |
| | pancreas, | | | | | |
| | endometrium | | | | | |
| EpCAM | epithelial | A24 | 20 | RYQLDPKFI | 703 | peptide |
| | cells | | | | | |
| EphA3 | many | DR11 | 25 | DVTFNIICKKCG | 704 | autologous |
| | | | | | | tumor cells |
| EZH2 | ubiquitous | A2 | 44 | FMVEDETVL | 705 | peptide |
| | (low level) | A2 | 44 | FINDEIFVEL | 706 | peptide |
| | | A24 | 20 | KYDCFLHPF | 707 | peptide |
| | | A24 | 20 | KYVGIEREM | 708 | peptide |
| FGF5 | brain, | A3 | 22 | NTYASPRFK^{f} | 709 | autologous |
| | kidney | | | | | tumor cells |
| glypican-3 | placental | A2 | 44 | FVGEFFTDV | 710 | peptide |
| | and | A24 | 20 | EYILSLEEL | 711 | peptide |
| | multiple | | | | | |
| | tissues | | | | | |
| G250/MN/ | stomach, | A2 | 44 | HLSTAFARV | 712 | peptide |
| CAIX | liver, | | | | | |
| | pancreas | | | | | |
| HER-2/ | ubiquitous | A2 | 44 | KIFGSLAFL | 713 | autologous |
| neu | (low level) | | | | | tumor cells |
| | | A2 | 44 | IISAVVGIL | 714 | peptide |
| | | A2 | 44 | ALCRWGLLL | 715 | peptide |
| | | A2 | 44 | ILHNGAYSL | 716 | peptide |
| | | A2 | 44 | RLLQETELV | 717 | peptide |
| | | A2 | 44 | VVLGVVFGI | 718 | peptide |
| | | A2 | 44 | YMIMVKCWMI | 719 | peptide |
| | | A2 | 44 | HLYQGCQVV | 720 | peptide |
| | | A2 | 44 | YLVPQQGFFC | 721 | peptide |
| | | A2 | 44 | PLQPEQLQV | 722 | peptide^{d} |
| | | A2 | 44 | TLEEITGYL | 723 | peptide^{d} |
| | | A2 | 44 | ALIHHNTHL | 724 | peptide^{d} |
| | | A2 | 44 | PLTSIISAV | 725 | peptide^{d} |
| | | A3 | 22 | VLRENTSPK | 726 | peptide |
| | | A24 | 20 | TYLPTNASL | 727 | peptide |
| HLA-DOB | B | A2 | 44 | FLLGLIFLL | 728 | peptide |
| | lymphocytes, | | | | | |
| | monocytes, | | | | | |
| | blood | | | | | |
| | cells, | | | | | |
| | adrenals, ... | | | | | |
| Hepsin | kidney, | A2 | 44 | SLLSGDWVL | 729 | peptide |
| | liver, skin, ... | A2 | 44 | GLQLGVQAV | 730 | peptide |
| | | A2 | 44 | PLTEYIQPV | 731 | peptide |
| IDO1 | lymph | A2 | 44 | ALLEIASCL | 732 | peptide |
| | nodes, | | | | | |
| | placenta, | | | | | |
| | and many | | | | | |
| | cell types | | | | | |
| | in the | | | | | |
| | course of | | | | | |
| | inflammatory | | | | | |
| | response | | | | | |
| IGF2B3 | ubiquitous | A2 | 44 | NLSSAEVVV | 733 | peptide |
| | (low level) | A3 | 44 | RLLVPTQFV | 734 | peptide |
| IL13Ralpha2 | | A2 | 44 | WLPFGFILI | 735 | peptide |
| Intestinal | liver, | B7 | 17 | SPRWWPTCL | 736 | autologous |
| carboxyl | intestine, | | | | | tumor cells |
| esterase | kidney | | | | | |
| alpha- | liver | A2 | 44 | GVALQTMKQ | 737 | adenovirus- |
| foetoprotein | | | | | | dendritic |
| | | | | | | cells |
| | | A2 | 44 | FMNKFIYEI | 738 | peptide |
| | | DR13 | 19 | QLAVSVILRV | 739 | peptide |
| Kallikrein 4 | prostate | A2 | 44 | FLGYLILGV | 740 | peptide |
| | and | DP4 | 75 | SVSESDTIRSISIAS | 741 | peptide |
| | ovarian | DR4 | 24 | LLANGRMPTVLQ | 742 | peptide |
| | cancer | | | CVN | | |
| | cancer | DR7 | 25 | RMPTVLQCVNVS | 743 | peptide |
| | | | | VVS | | |
| KIF20A | ubiquitous | A2 | 44 | LLSDDDVVV | 744 | peptide |
| | (low level) | A2 | 44 | AQPDTAPLPV | 745 | peptide |
| | | A2 | 44 | CIAEQYHTV | 746 | peptide |
| Lengsin | eye lens | A2 | 44 | FLPEFGISSA | 747 | peptide |
| | and low | | | | | |
| | level in | | | | | |
| | multiple | | | | | |
| | tissues | | | | | |
| M-CSF | liver, | B35 | 20 | LPAVVGLSPGEQEY | 748 | autologous |
| | kidney | | | | | tumor cells |
| MCSP | endothelial | DR11 | 25 | VGQDVSVLFRVT | 749 | peptide |
| | cells, | | | GALQ | | |
| | chondrocytes, | | | | | |
| | smooth | | | | | |
| | muscle | | | | | |
| | cells | | | | | |
| mdm-2 | ubiquitous | A2 | 44 | VLFYLGQY | 750 | tumor |
| | (brain, | | | | | lysate- |
| | muscle, | | | | | pulsed |
| | lung) | | | | | APCs |
| Meloe | ubiquitous | A2 | 44 | TLNDECWPA | 751 | tumor- |
| | (low level) | | | | | infiltrating |
| | | | | | | lymphocytes |
| | | DQ2 | 41 | ERISSTLNDECWPA | 752 | peptide/protein |
| | | DQ6 | 63 | FGRLQGISPKI | 753 | peptide |
| | | DR1 | 18 | TSREQFLPSEGAA | 754 | peptide/protein |
| | | DR11 | 25 | CPPWHPSERISSTL | 755 | peptide |
| Midkine | ubiquitous | A2 | 44 | ALLALTSAV | 756 | peptide |
| | (low level) | A2 | 44 | AQCQETIRV | 757 | peptide |
| | | DR4 | 24 | LTLLALLALTSAV | 758 | peptide |
| | | AK | | | | |
| MMP-2 | ubiquitous | A2 | 44 | GLPPDVQRV^{h} | 759 | autologous |
| | | | | | | tumor cells |
| MMP-7 | ubiquitous | A3 | 22 | SLFPNSPKWTSK | 760 | peptide |
| | (low level) | | | | | |
| MUC1 | glandular | A2 | 44 | STAPPVHNV | 761 | peptide |
| | epithelia | A2 | 44 | LLLLTVLTV | 762 | peptide |
| | | DR3 | 21 | PGSTAPPAHGVT | 763 | peptide |
| MUC5AC | surface | A24 | 20 | TCQPTCRSL | 764 | peptide |
| | mucosal | | | | | |
| | cells, | | | | | |
| | respiratory | | | | | |
| | tract, and | | | | | |
| | stomach | | | | | |
| | epithelia | | | | | |
| p53 | ubiquitous | A2 | 44 | LLGRNSFEV | 765 | peptide |
| | (low level) | A2 | 44 | RMPEAAPPV | 766 | peptide |
| | | B46 | 0.1 | SQKTYQGSY | 767 | autologous |
| | | | | | | tumor cells |
| | | DP5 | 3 | PGTRVRAMAIYKQ | 768 | peptide |
| | | DR14 | 6 | HLIRVEGNLRVE | 769 | peptide |
| PAX5 | hemopoietic | A2 | 44 | TLPGYPPHV | 770 | peptide |
| | system | | | | | |
| PBF | ovary, | B55 | 4 | CTACRWKKACQR | 771 | autologous |
| | pancreas, | | | | | tumor cells |
| | spleen, | | | | | |
| | liver | | | | | |
| PRAME | testis, | A2 | 44 | VLDGLDVLL | 772 | peptide |
| | ovary, | A2 | 44 | SLYSFPEPEA | 773 | peptide |
| | endometrium, | A2 | 44 | ALYVDSLFFL | 774 | peptide |
| | adrenals | A2 | 44 | SLLQHLIGL | 775 | peptide |
| | | A24 | 20 | LYVDSLFFL^{c} | 776 | autologous |
| | | | | | | tumor cells |
| PSMA | prostate, | A24 | 20 | NYARTEDFF | 777 | peptide |
| | CNS, liver | | | | | |
| RAGE-1 | retina | A2 | 44 | LKLSGVVRL | 778 | peptide |
| | | A2 | 44 | PLPPARNGGL^{g} | 779 | peptide |
| | | B7 | 17 | SPSSNRIRNT | 780 | autologous |
| | | | | | | tumor cells |
| RGS5 | heart, | A2 | 44 | LAALPHSCL | 781 | peptide |
| | skeletal | A3 | 22 | GLASFKSFLK | 782 | peptide |
| | muscle, | | | | | |
| | pericytes | | | | | |
| RhoC | ubiquitous | A3 | 22 | RAGLQVRKNK | 783 | peptide |
| | (low level) | | | | | |
| RNF43 | | A2 | 44 | ALWPWLLMA(T) | 784 | peptide |
| | | A24 | 20 | NSQPVWLCL | 785 | peptide |
| RU2AS | testis, | B7 | 17 | LPRWPPPQL | 786 | autologous |
| | kidney, | | | | | tumor cells |
| | bladder | | | | | |
| secernin 1 | ubiquitous | A2 | 44 | KMDAEHPEL | 787 | peptide |
| SOX10 | ubiquitous | A2 | 44 | AWISKPPGV | 788 | tumor- |
| | (low level) | | | | | infiltrating |
| | | | | | | lymphocytes |
| | | A2 | 44 | SAWISKPPGV | 789 | tumor- |
| | | | | | | infiltrating |
| | | | | | | lymphocytes |
| STEAP1 | prostate | A2 | 44 | MIAVFLPIV | 790 | peptide |
| | | A2 | 44 | HQQYFYKIPILVINK | 791 | peptide |
| survivin | ubiquitous | A2 | 44 | ELTLGEFLKL | 792 | peptide/ |
| | | | | | | protein |
| | ubiquitous | DR1 | 18 | TLGEFLKLDRERA | 793 | peptide/ |
| | | | | KN | | protein |
| Telomerase | testis, | A2 | 44 | ILAKFLHWL^{e} | 794 | peptide |
| | thymus, | A2 | 44 | RLVDDFLLV | 795 | peptide |
| | bone | DR7 | 25 | RPGLLGASVLGL | 796 | peptide |
| | marrow, | | | DDI | | |
| | lymph | DR11 | 25 | LTDLQPYMRQFV | 797 | peptide |
| | nodes | | | AHL | | |
| TPBG | multiple | A2 | 44 | RLARLALVL | 798 | peptide |
| | tissues | | | | | |
| | (esophagus, | | | | | |
| | bladder, ...) | | | | | |
| VEGF | ubiquitous | B27 | 7 | SRFGGAVVR | 799 | peptide |
| | (low level) | | | | | |
| WT1 | testis, | A1 | 26 | TSEKRPFMCAY | 800 | peptide |
| | ovary, | A24 | 20 | CMTWNQMNL | 801 | peptide |
| | bone | DP5 | 3 | LSHLQMHSRKH | 802 | peptide |
| | marrow, | DP5 | 3 | KRYFKLSHLQMH | 803 | peptide |
| | spleen | | | SRKH | | |
| | | DR4 | 24 | KRYFKLSHLQMH | 804 | peptide |
| | | | | SRKH | | |

^{b}Frequency in Caucasians, based on (1) Marsh SGE, Parham P, Barber LD. The HLA Factsbook (Academic Press, 2000) for HLA-A, B, C, and DR, and (2) Colombani J. HLA, Fonctions immunitaires et applications médicales (John Libbey Eurotext, 1993) for HLA-DP and DR.
^{c}The antigen is recognized by CTLs bearing an NK inhibitory receptor that prevents lysis of cells expressing certain HLA-C molecules.
^{d}A variant peptide with a Y in position 1 was used for T cell stimulation.
^{e}Poorly or not processed (Parkhurst, 2004; Ayyoub, 2001).
^{f}The peptide is composed of two non-contiguous fragments that are spliced.
^{g}Alternative transcript.
^{h}MMP-2 is expressed ubiquitously but melanoma cells cross-present, in an αvβ3-dependent manner, an antigen derived from secreted MMP-2.
ⁱThe epitope is located in the untranslated region.

The cytoplasmic domain (also referred to as “intracellular signaling domain”) of the CAR molecule of the invention is responsible for activation of at least one of the normal effector functions of the immune cell in which the CAR has been placed in.

The term “effector function” refers to a specialized function of a cell. Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

Preferred examples of intracellular signaling domains for use in the CAR molecule of the invention include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

It is known that signals generated through the TCR alone are insufficient for full activation of the T cell and that a secondary or co-stimulatory signal is also required. Thus, T cell activation can be mediated by two distinct classes of cytoplasmic signaling sequence: those that initiate antigen-dependent primary activation through the TCR (primary cytoplasmic signaling sequences) and those that act in an antigen-independent manner to provide a secondary or co-stimulatory signal (secondary cytoplasmic signaling sequences).

Primary cytoplasmic signaling sequences regulate primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs (ITAMs).

Examples of ITAM containing primary cytoplasmic signaling sequences that are of particular use in the invention include those derived from TCR zeta, FcR gamma, FcR beta, CD3 gamma, CD3 delta, CD3 epsilon, CD5, CD22, CD79a, CD79b, and CD66d. It is particularly preferred that cytoplasmic signaling molecule in the CAR of the invention comprises a cytoplasmic signaling sequence derived from CD3 zeta.

In a preferred embodiment, the cytoplasmic domain of the CAR can be designed to comprise the CD3-zeta signaling domain by itself or combined with any other desired cytoplasmic domain(s) useful in the context of the CAR of the invention. For example, the cytoplasmic domain of the CAR can comprise a CD3 zeta chain portion and a costimulatory signaling region. The costimulatory signaling region refers to a portion of the CAR comprising the intracellular domain of a costimulatory molecule. A co-stimulatory molecule is a cell surface molecule other than an antigen receptor or their ligands that is required for an efficient response of lymphocytes to an antigen. Examples of such molecules include CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83, and the like. Thus, while the invention in exemplified primarily with 4-1BB as the co-stimulatory signaling element, other costimulatory elements are within the scope of the invention.

According to some embodiments of the invention, the intracellular domain comprises, a co-stimulatory signaling region and a zeta chain portion. The co-stimulatory signaling region refers to a portion of the CAR molecule comprising the intracellular domain of a co-stimulatory molecule. Co-stimulatory molecules are cell surface molecules other than antigen receptors or their ligands that are required for an efficient response of lymphocytes to antigen.

“Co-stimulatory ligand,” as the term is used herein, includes a molecule on an antigen presenting cell [e.g., an aAPC (artificial antigen presenting cell), dendritic cell, B cell, and the like] that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including, but not limited to, proliferation, activation, differentiation, and the like. A co-stimulatory ligand can include, but is not limited to, CD7, B7-1 (CD80), B7-2 (CD86), PD-L1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as, but not limited to, CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds with CD83.

A “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to an MHC class 1 molecule, BTLA and a Toll ligand receptor.

A “co-stimulatory signal”, as used herein, refers to a signal, which in combination with a primary signal, such as TCR/CD3 ligation, leads to T cell proliferation and/or upregulation or down regulation of key molecules.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-β, and/or reorganization of cytoskeletal structures, and the like.

A “stimulatory molecule,” as the term is used herein, means a molecule on a T cell that specifically binds with a cognate stimulatory ligand present on an antigen presenting cell.

A “stimulatory ligand,” as used herein, means a ligand that when present on an antigen presenting cell (e.g., an aAPC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter cilia, an MHC Class I molecule loaded with a peptide, an anti-CD3 antibody, a superagonist anti-CD28 antibody, and a superagonist anti-CD2 antibody.

With respect to the cytoplasmic domain, the CAR molecule of some embodiments of the invention can be designed to comprise the CD28 and/or 4-1BB signaling domain by itself or be combined with any other desired cytoplasmic domain(s) useful in the context of the CAR molecule of some embodiments of the invention. In one embodiment, the cytoplasmic domain of the CAR can be designed to further comprise the signaling domain of CD3-zeta. For example, the cytoplasmic domain of the CAR can include but is not limited to CD3-zeta, 4-1BB and CD28 signaling modules and combinations thereof.

According to some embodiments of the invention, the intracellular domain comprises at least one, e.g., at least two, at least three, at least four, at least five, e.g., at least six of the polypeptides selected from the group consisting of: CD3 (CD247, CD3z), CD28, 41BB, ICOS, OX40, and CD137.

According to some embodiments of the invention, the intracellular domain comprises the CD3ζ-chain [CD247 molecule, also known as “CD3-ZETA” and “CD3z”; GenBank Accession NOs. NP_000725.1 (SEQ ID NO:86) and NP_932170.1 (SEQ ID NO:87)], which is the primary transmitter of signals from endogenous TCRs.

According to some embodiments of the invention, the intracellular domain comprises various co-stimulatory protein receptors to the cytoplasmic tail of the CAR to provide additional signals to the T cell (second generation CAR). Examples include, but are not limited to, CD28 [e.g., GenBank Accession Nos. NP_001230006.1 (SEQ ID NO:88), NP_001230007.1 (SEQ ID NO:89), NP_006130.1 (SEQ ID NO:90)], 4-1BB [tumor necrosis factor receptor superfamily, member 9 (TNFRSF9), also known as “CD137”, e.g., GenBank Accession No. NP_001552.2 (SEQ ID NO:91)], and ICOS [inducible T-cell co-stimulator, e.g., GenBank Accession No. NP_036224.1 (SEQ ID NO:92)]. Preclinical studies have indicated that the second generation of CAR designs improves the antitumor activity of T cells.

According to some embodiments of the invention, the intracellular domain comprises multiple signaling domains, such as CD3z-CD28-41BB or CD3z-CD28-OX40, to further augment potency. The term “OX40” refers to the tumor necrosis factor receptor superfamily, member 4 (TNFRSF4), e.g., GenBank Accession No. NP_003318.1 (SEQ ID NO:93) (“third-generation” CARs).

According to some embodiments of the invention, the intracellular domain comprises CD28-CD3z, CD3z, CD28-CD137-CD3z. The term “CD137” refers to tumor necrosis factor receptor superfamily, member 9 (TNFRSF9), e.g., GenBank Accession No. NP_001552.2 (SEQ ID NO:91).

According to some embodiments of the invention, when the CAR molecule is designed for a natural killer cell, then the signaling domain can be CD28 and/or CD3. The transmembrane domain may be derived either from a natural or from a synthetic source. Where the source is natural, the domain may be derived from any membrane-bound or transmembrane protein. Transmembrane regions of particular use in this invention may be derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154. Alternatively the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

According to some embodiments of the invention, the transmembrane domain comprised in the CAR molecule of some embodiments of the invention is a transmembrane domain that is naturally associated with one of the domains in the CAR. According to some embodiments of the invention, the transmembrane domain can be selected or modified by amino acid substitution to avoid binding of such domains to the transmembrane domains of the same or different surface membrane proteins to minimize interactions with other members of the receptor complex.

According to some embodiments of the invention, the transmembrane domain is the CD8a hinge domain. In one embodiment, the CD8 hinge domain comprises the nucleic acid sequence of SEQ ID NO:34. In one embodiment, the CD8 hinge domain comprises the nucleic acid sequence that encodes the amino acid sequence of SEQ ID NO: 35).

According to some embodiments, between the extracellular domain and the transmembrane domain of the CAR molecule, or between the cytoplasmic domain and the transmembrane domain of the CAR molecule, there may be incorporated a spacer domain. As used herein, the term “spacer domain” generally means any oligo- or polypeptide that functions to link the transmembrane domain to, either the extracellular domain or, the cytoplasmic domain in the polypeptide chain. A spacer domain may comprise up to 300 amino acids, preferably 10 to 100 amino acids and most preferably 25 to 50 amino acids.

According to an aspect of some embodiments of the invention there is provided an isolated polynucleotide comprising a nucleic acid sequence encoding the molecule of some embodiments of the invention.

As used herein the term “polynucleotide” refers to a single or double stranded nucleic acid sequence which is isolated and provided in the form of an RNA sequence, a complementary polynucleotide sequence (cDNA), a genomic polynucleotide sequence and/or a composite polynucleotide sequences (e.g., a combination of the above).

The term “isolated” refers to at least partially separated from the natural environment e.g., from a cell, or from a tissue, e.g., from a human body.

The isolated polynucleotide can be obtained using recombinant methods known in the art, such as, for example by screening libraries from cells expressing the gene, by deriving the gene from a vector known to include the same, or by isolating directly from cells and tissues containing the same, using standard techniques.

Alternatively, the gene of interest can be produced synthetically, rather than cloned.

According to an aspect of some embodiments of the invention there is provided a nucleic acid construct comprising an isolated polynucleotide comprising a nucleic acid sequence encoding the molecule of some embodiments of the invention and a cis-acting regulatory element for directing transcription of the isolated polynucleotide in a host cell.

Thus, the expression of natural or synthetic nucleic acids encoding the CAR molecule of the invention is typically achieved by operably linking a nucleic acid encoding the CAR polypeptide or portions thereof to a cis-acting regulatory element (e.g., a promoter sequence), and incorporating the construct into an expression vector. The nucleic acid construct of the invention may also include an enhancer, a transcription and translation initiation sequence, transcription and translation terminator and a polyadenylation signal, a 5′ LTR, a tRNA binding site, a packaging signal, an origin of second-strand DNA synthesis, and a 3′ LTR or a portion thereof; additional polynucleotide sequences that allow, for example, the translation of several proteins from a single mRNA such as an internal ribosome entry site (IRES) and sequences for genomic integration of the promoter-chimeric polypeptide; sequences engineered to enhance stability, production, purification, yield or toxicity of the expressed peptide.

Enhancers regulate the frequency of transcriptional initiation. Typically, promoter elements are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1.alpha. (EF-1.alpha.). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

The isolated polynucleotide of the invention can be cloned into a number of types of vectors. For example, the isolated polynucleotide can be cloned into a vector including, but not limited to a plasmid, a phagemid, a phage derivative, an animal virus, and a cosmid. Vectors of particular interest include expression vectors, replication vectors, probe generation vectors, and sequencing vectors.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p205. Other exemplary vectors include pMSG, pAV009/A⁺, pMT010/A⁺, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

Currently preferred in vivo or in vitro nucleic acid transfer techniques include transfection with viral or non-viral constructs, such as adenovirus, lentivirus, Herpes simplex I virus, or adeno-associated virus (AAV). Recombinant viral vectors offer advantages such as lateral infection and targeting specificity. Introduction of nucleic acids by viral infection offers several advantages over other methods such as lipofection and electroporation, since higher transfection efficiency can be obtained due to the infectious nature of viruses.

Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

According to some embodiments of the invention, the nucleic acid construct of the invention is a viral vector.

Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo.

The nucleic acid construct of some embodiments of the invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the invention provides a gene therapy vector.

In order to assess the expression of a CAR polypeptide or portions thereof, the nucleic acid construct to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tel et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Various methods can be used to introduce the nucleic acid construct of the invention into a host cell, e.g., mammalian, bacterial, yeast, or insect cell. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, physical, chemical, or biological means (e.g., stable or transient transfection, lipofection, electroporation and infection with recombinant viral vectors). In addition, see U.S. Pat. Nos. 5,464,764 and 5,487,992 for positive-negative selection methods.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). A preferred method for the introduction of a polynucleotide into a host cell is calcium phosphate transfection.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus 1, adenoviruses and adeno-associated viruses, and the like. See, for example, U.S. Pat. Nos. 5,350,674 and 5,585,362.

In the case where a non-viral delivery system is utilized, an exemplary delivery vehicle is a liposome.

“Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”); and other lipids may be obtained from Avanti Polar Lipids, Inc, (Birmingham, Ala.). Additionally or alternatively, the DOTMA, DOPE, and DC-Chol [Tonkinson et al., Cancer Investigation, 14(1): 54-65 (1996)] lipids can be used. Stock solutions of lipids in chloroform or chloroform/methanol can be stored at about −20.degree. C. Chloroform is used as the only solvent since it is more readily evaporated than methanol.

Regardless of the method used to introduce exogenous nucleic acids into a host cell, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

Thus, according to an aspect of some embodiments of the invention there is provided an isolated cell comprising the polynucleotide of some embodiments of the invention or the nucleic acid construct of some embodiments of the invention.

According to some embodiments of the invention, the cell is a T cell, a natural killer cell, a cell that exerts effector killing function on a target cell, a cell that exerts a suppressive effect on effector T cells, an engineered cell with an effector killing function or an engineered cell with a suppressive function.

According to some embodiments of the invention, the cell is a T cell.

According to some embodiments of the invention, the cell is a natural killer (NK) cell.

According to some embodiments of the invention, the natural killer cell is used to target cancer, viral and/or bacterial antigen(s).

According to some embodiments of the invention, the natural killer cell is used to treat a pathology caused by or associated with a viral infection, bacterial infection or cancer.

According to some embodiments of the invention, the T cell is a cytotoxic T cell (effector T cell).

According to some embodiments of the invention, the cytotoxic T cell (effector T cell) is used to target cancer, viral and/or bacterial antigen(s). According to some embodiments of the invention, the cytotoxic T cell is used to treat a pathology caused by or associated with a viral infection, bacterial infection or cancer.

According to some embodiments of the invention, the T cell comprises a Treg (T regulatory cell).

According to some embodiments of the invention, the Treg is used to target auto-immune antigen(s).

According to some embodiments of the invention, the Treg is used to treat an autoimmune disease.

According to some embodiments of the invention, the T cell comprises a CD4 T cell.

According to some embodiments of the invention, the T cell comprises a CD8 T cell.

Sources of T Cells

Prior to expansion and genetic modification of the T cells of the invention, a source of T cells is obtained from a subject. T cells can be obtained from a number of sources, including peripheral blood mononuclear cells (PBMCs), bone marrow, lymph node tissue, cord blood, thymus tissue, tissue from a site of infection, ascites, pleural effusion, spleen tissue, and tumors.

According to some embodiments of the invention, the T cell is obtained from peripheral blood mononuclear cells (PBMCs).

In certain embodiments of the present invention, any number of T cell lines available in the art, may be used. In certain embodiments of the present invention, T cells can be obtained from a unit of blood collected from a subject using any number of techniques known to the skilled artisan, such as Ficoll™ separation. In one preferred embodiment, cells from the circulating blood of an individual are obtained by apheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or may lack many if not all divalent cations. Again, surprisingly, initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca.sup.2+-free, Mg.sup.2+-free PBS, PlasmaLyte A, or other saline solution with or without buffer. Alternatively, the undesirable components of the apheresis sample may be removed and the cells directly resuspended in culture media.

In another embodiment, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3.sup.+, CD28.sup.+, CD4.sup.+, CD8.sup.+, CD45RA.sup.+, and CD45RO.sup.+ T cells, can be further isolated by positive or negative selection techniques. For example, in one embodiment, T cells are isolated by incubation with anti-CD3/anti-CD28 (i.e., 3.times.28)-conjugated beads, such as DYNABEADS® M-450 CD3/CD28 T, for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In one preferred embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immune-compromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process.

Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used in the context of this invention. In certain embodiments, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD11b, CD 16, HLA-DR, and CD8. In certain embodiments, it may be desirable to enrich for or positively select for regulatory T cells which typically express CD4⁺, CD25⁺, CD62L^{hi}, GITR⁺, and FoxP3⁺. Alternatively, in certain embodiments, T regulatory cells are depleted by anti-CD25 conjugated beads or other similar method of selection.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc.). Such populations of cells may have therapeutic value and would be desirable to obtain. For example, using high concentration of cells allows more efficient selection of CD8⁺ T cells that normally have weaker CD28 expression.

In a related embodiment, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4⁺ T cells express higher levels of CD28 and are more efficiently captured than CD8⁺ T cells in dilute concentrations. In one embodiment, the concentration of cells used is 5×10⁶/ml. In other embodiments, the concentration used can be from about 1×10⁵/ml to 1×10⁶/ml, and any integer value in between.

In other embodiments, the cells may be incubated on a rotator for varying lengths of time at varying speeds at either 2-10° C. or at room temperature.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° C. per minute and stored in the vapor phase of a liquid nitrogen storage tank. Other methods of controlled freezing may be used as well as uncontrolled freezing immediately at −20° C. or in liquid nitrogen.

In certain embodiments, cryopreserved cells are thawed and washed as described herein and allowed to rest for one hour at room temperature prior to activation using the methods of the present invention.

Also contemplated in the context of the invention is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in T cell therapy for any number of diseases or conditions that would benefit from T cell therapy, such as those described herein. In one embodiment a blood sample or an apheresis is taken from a generally healthy subject. In certain embodiments, a blood sample or an apheresis is taken from a generally healthy subject who is at risk of developing a disease, but who has not yet developed a disease, and the cells of interest are isolated and frozen for later use. In certain embodiments, the T cells may be expanded, frozen, and used at a later time. In certain embodiments, samples are collected from a patient shortly after diagnosis of a particular disease as described herein but prior to any treatments. In a further embodiment, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation. These drugs inhibit either the calcium dependent phosphatase calcineurin (cyclosporine and FK506) or inhibit the p70S6 kinase that is important for growth factor induced signaling (rapamycin) (Liu et al., Cell 66:807-815, 1991; Henderson et al., Immun. 73:316-321, 1991; Bierer et al., Curr. Opin. Immun. 5:763-773, 1993). In a further embodiment, the cells are isolated for a patient and frozen for later use in conjunction with (e.g., before, simultaneously or following) bone marrow or stem cell transplantation, T cell ablative therapy using either chemotherapy agents such as, fludarabine, external-beam radiation therapy (XRT), cyclophosphamide, or antibodies such as OKT3 or CAMPATH. In another embodiment, the cells are isolated prior to and can be frozen for later use for treatment following B-cell ablative therapy such as agents that react with CD20, e.g., Rituxan.

In a further embodiment of the present invention, T cells are obtained from a patient directly following treatment. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including T cells, natural killer cells, dendritic cells, or other cells of the hematopoietic lineage, during this recovery phase. Further, in certain embodiments, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

According to some embodiments of the invention, the T cell is autologous to the subject.

As used herein, the term “autologous” is meant to refer to any material derived from the same individual to which it is later to be re-introduced into the individual.

According to some embodiments of the invention, the T cell is semi-autologous to the subject.

According to some embodiments of the invention, the T cell is non-autologous (e.g., allogeneic) to the subject.

“Allogeneic” refers to a graft derived from a different individual.

Activation and Expansion of T Cells

“Activation”, as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

Whether prior to or after genetic modification of the T cells to express a desirable CAR molecule, the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, the T cells of the invention are expanded by contact with a surface having attached thereto an agent that stimulates a CD3/TCR complex associated signal and a ligand that stimulates a co-stimulatory molecule on the surface of the T cells. In particular, T cell populations may be stimulated as described herein, such as by contact with an anti-CD3 antibody, or antigen-binding fragment thereof, or au anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4⁺ T cells or CD8⁺ T cells, an anti-CD3 antibody and an anti-CD28 antibody. Examples of an anti-CD28 antibody include 9.3, B-T3, XR-CD28 (Diaclone, Besancon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol. Meth. 227(1-2):53-63, 1999).

In certain embodiments, the primary stimulatory signal and the co-stimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In one embodiment, the agent providing the co-stimulatory signal is bound to a cell surface and the agent providing the primary activation signal is in solution or coupled to a surface. In certain embodiments, both agents can be in solution. In another embodiment, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in the present invention.

In one embodiment, the two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the co-stimulatory signal is an anti-CD28 antibody or antigen-binding fragment thereof; and both agents are co-immobilized to the same bead in equivalent molecular amounts. In one embodiment, a 1:1 ratio of each antibody bound to the beads for CD4⁺ T cell expansion and T cell growth is used. In certain aspects of the present invention, a ratio of anti CD3:CD28 antibodies bound to the beads is used such that an increase in T cell expansion is observed as compared to the expansion observed using a ratio of 1:1. In one particular embodiment an increase of from about 1 to about 3 fold is observed as compared to the expansion observed using a ratio of 1:1. In one embodiment, the ratio of CD3:CD28 antibody bound to the beads ranges from 100:1 to 1:100 and all integer values there between. In one aspect of the present invention, more anti-CD28 antibody is bound to the particles than anti-CD3 antibody, i.e., the ratio of CD3:CD28 is less than one. In certain embodiments of the invention, the ratio of anti CD28 antibody to anti CD3 antibody bound to the beads is greater than 2:1. In one particular embodiment, a 1:100 CD3:CD28 ratio of antibody bound to beads is used. In another embodiment, a 1:75 CD3:CD28 ratio of antibody bound to beads is used. In a further embodiment, a 1:50 CD3:CD28 ratio of antibody bound to beads is used. In another embodiment, a 1:30 CD3:CD28 ratio of antibody bound to beads is used. In one preferred embodiment, a 1:10 CD3:CD28 ratio of antibody bound to beads is used. In another embodiment, a 1:3 CD3:CD28 ratio of antibody bound to the beads is used. In yet another embodiment, a 3:1 CD3:CD28 ratio of antibody bound to the beads is used.

Ratios of particles to cells from 1:500 to 500:1 and any integer values in between may be used to stimulate T cells or other target cells. As those of ordinary skill in the art can readily appreciate, the ratio of particles to cells may depend on particle size relative to the target cell. For example, small sized beads could only bind a few cells, while larger beads could bind many. In certain embodiments the ratio of cells to particles ranges from 1:100 to 100:1 and any integer values in-between and in further embodiments the ratio comprises 1:9 to 9:1 and any integer values in between, can also be used to stimulate T cells. The ratio of anti-CD3- and anti-CD28-coupled particles to T cells that result in T cell stimulation can vary as noted above, however certain preferred values include 1:100, 1:50, 1:40, 1:30, 1:20, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, and 15:1 with one preferred ratio being at least 1:1 particles per T cell. In one embodiment, a ratio of particles to cells of 1:1 or less is used. In one particular embodiment, a preferred particle:cell ratio is 1:5. In further embodiments, the ratio of particles to cells can be varied depending on the day of stimulation. For example, in one embodiment, the ratio of particles to cells is from 1:1 to 10:1 on the first day and additional particles are added to the cells every day or every other day thereafter for up to 10 days, at final ratios of from 1:1 to 1:10 (based on cell counts on the day of addition). In one particular embodiment, the ratio of particles to cells is 1:1 on the first day of stimulation and adjusted to 1:5 on the third and fifth days of stimulation. In another embodiment, particles are added on a daily or every other day basis to a final ratio of 1:1 on the first day, and 1:5 on the third and fifth days of stimulation. In another embodiment, the ratio of particles to cells is 2:1 on the first day of stimulation and adjusted to 1:10 on the third and fifth days of stimulation. In another embodiment, particles are added on a daily or every other day basis to a final ratio of 1:1 on the first day, and 1:10 on the third and fifth days of stimulation. One of skill in the art will appreciate that a variety of other ratios may be suitable for use in the present invention. In particular, ratios will vary depending on particle size and on cell size and type.

In further embodiments of the present invention, the cells, such as T cells, are combined with agent-coated beads, the beads and the cells are subsequently separated, and then the cells are cultured. In an alternative embodiment, prior to culture, the agent-coated beads and cells are not separated but are cultured together. In a further embodiment, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached to contact the T cells. In one embodiment the cells (for example, 10⁴ to 10⁹ T cells) and beads (for example, DYNABEADS® M-450 CD3/CD28 T paramagnetic beads at a ratio of 1:1) are combined in a buffer, preferably PBS (without divalent cations such as, calcium and magnesium). Again, those of ordinary skill in the art can readily appreciate any cell concentration may be used. For example, the target cell may be very rare in the sample and comprise only 0.01% of the sample or the entire sample (i.e., 100%) may comprise the target cell of interest. Accordingly, any cell number is within the context of the present invention. In certain embodiments, it may be desirable to significantly decrease the volume in which particles and cells are mixed together (i.e., increase the concentration of cells), to ensure maximum contact of cells and particles. For example, in one embodiment, a concentration of about 2 billion cells/ml is used. In another embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells. Such populations of cells may have therapeutic value and would be desirable to obtain in certain embodiments. For example, using high concentration of cells allows more efficient selection of CD8⁺ T cells that normally have weaker CD28 expression.

In one embodiment of the present invention, the mixture may be cultured for several hours (about 3 hours) to about 14 days or any hourly integer value in between. In another embodiment, the mixture may be cultured for 21 days. In one embodiment of the invention the beads and the T cells are cultured together for about eight days. In another embodiment, the beads and T cells are cultured together for 2-3 days. Several cycles of stimulation may also be desired such that culture time of T cells can be 60 days or more. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-.gamma., IL-4, IL-7, GM-CSF, IL-10, IL-12, IL-15, TGFβ, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

T cells that have been exposed to varied stimulation times may exhibit different characteristics. For example, typical blood or apheresed peripheral blood mononuclear cell products have a helper T cell population (T_{H}, CD4⁺) that is greater than the cytotoxic or suppressor T cell population (Tc, CD8⁺). Ex vivo expansion of T cells by stimulating CD3 and CD28 receptors produces a population of T cells that prior to about days 8-9 consists predominately of T_{H} cells, while after about days 8-9, the population of T cells comprises an increasingly greater population of T_{C} cells. Accordingly, depending on the purpose of treatment, infusing a subject with a T cell population comprising predominately of T_{H} cells may be advantageous. Similarly, if an antigen-specific subset of T_{C} cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

Thus, according to an aspect of some embodiments of the invention there is provided a pharmaceutical composition comprising the CAR molecule of some embodiments of the invention, the isolated polynucleotide of some embodiments of the invention, the nucleic acid construct of some embodiments of the invention and/or the cell of some embodiments of the invention and a pharmaceutically acceptable carrier.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the CAR molecule of some embodiments of the invention the isolated polynucleotide of some embodiments of the invention, the nucleic acid construct of some embodiments of the invention and/or the cell of some embodiments of the invention accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intravenous, intraperitoneal, intranasal, or intraocular injections.

Alternately, one may administer the pharmaceutical composition in a local rather than systemic manner, for example, via injection of the pharmaceutical composition directly into a tissue region of a patient.

Pharmaceutical compositions of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

The administration of the pharmaceutical composition may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous (i.v.) injection, or intraperitoneally. In one embodiment, the pharmaceutical composition of the present invention is administered to a patient by intradermal or subcutaneous injection. In another embodiment, the pharmaceutical composition of the present invention is preferably administered by i.v. injection. The pharmaceutical composition may be injected directly into a tumor, lymph node, or site of infection.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use. The pharmaceutical composition of the invention may also be formulated in rectal compositions such as suppositories or retention enemas, using, e.g., conventional suppository bases such as cocoa butter or other glycerides.

Pharmaceutical compositions suitable for use in context of the invention include compositions wherein the active ingredients are contained in an amount effective to achieve the intended purpose. More specifically, a therapeutically effective amount means an amount of active ingredients effective to prevent, alleviate or ameliorate symptoms of a pathology or prolong the survival of the subject being treated.

Determination of a therapeutically effective amount is well within the capability of those skilled in the art, especially in light of the detailed disclosure provided herein.

When “an immunologically effective amount”, “an anti-tumor effective amount”, “an tumor-inhibiting effective amount”, or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the T cells or NK cells described herein may be administered at a dosage of 10⁴ to 10⁹ cells/kg body weight, preferably 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

For example, the effect of the active ingredients (e.g., the isolated polynucleotide of some embodiments of the invention, the nucleic acid construct of some embodiments of the invention or the cell of some embodiments of the invention) on the pathology can be evaluated by monitoring the level of markers, e.g., hormones, glucose, peptides, carbohydrates, etc. in a biological sample of the treated subject using well known methods.

In certain embodiments, it may be desired to administer activated T cells to a subject and then subsequently redraw blood (or have an apheresis performed), activate T cells therefrom according to the present invention, and reinfuse the patient with these activated and expanded T cells. This process can be carried out multiple times every few weeks. In certain embodiments, T cells can be activated from blood draws of from 10 cc to 400 cc. In certain embodiments, T cells are activated from blood draws of 20 cc, 30 cc, 40 cc, 50 cc, 60 cc, 70 cc, 80 cc, 90 cc, or 100 cc. Not to be bound by theory, using this multiple blood draw/multiple reinfusion protocol may serve to select out certain populations of T cells.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide plasma or brain levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

According to some embodiments of the invention, the therapeutic agent of the invention can be provided to the subject in conjunction with other drug(s) designed for treating the pathology [combination therapy, (e.g., before, simultaneously or following)].

In certain embodiments of the present invention, cells activated and expanded using the methods described herein, or other methods known in the art where T cells are expanded to therapeutic levels, are administered to a patient in conjunction with any number of relevant treatment modalities, including but not limited to treatment with agents such as antiviral therapy, cidofovir and interleukin-2, Cytarabine (also known as ARA-C) or natalizumab treatment for MS patients or efalizumab treatment for psoriasis patients or other treatments for PML patients. In further embodiments, the T cells of the invention may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. These drugs inhibit either the calcium dependent phosphatase calcineurin (cyclosporine and FK506) or inhibit the p70S6 kinase that is important for growth factor induced signaling (rapamycin) (Liu et al., Cell 66:807-815, 1991; Henderson et al., Immun. 73:316-321, 1991; Bierer et al., Curr. Opin. Immun. 5:763-773, 1993). In a further embodiment, the cell compositions of the present invention are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the cell compositions of the present invention are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

The combination therapy may increase the therapeutic effect of the agent of the invention in the treated subject.

Compositions of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as if further detailed above.

According to an aspect of some embodiments of the invention, there is provided an in vitro method of generating a medicament for treating a pathology in a subject in need thereof, comprising:

(a) obtaining T cells or natural killer (NK) cells,

(b) transducing the T cells or the natural killer cells with the nucleic acid construct of some embodiments of the invention, wherein binding of the molecule to the antigen elicits a therapeutic response by the T cells or the natural killer of the subject,

thereby generating the medicament for treating the pathology.

According to some embodiments of the invention, the T cells or the natural killer cells are obtained from peripheral blood mononuclear cells (PBMCs) of the subject in need thereof.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

According to an aspect of some embodiments of the invention, there is provided a method of treating a pathology in a subject in need thereof, comprising administering the medicament resultant of the method of some embodiments of the invention in the subject, thereby treating the pathology in the subject.

The term “treating” refers to inhibiting, preventing or arresting the development of a pathology (disease, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the term “subject” includes mammals, preferably human beings at any age which suffer from the pathology.

The pathology can be, but is not limited to, cancer, viral infection, bacterial and parasitic infections, and/or an autoimmune disease.

According to some embodiments of the invention, the pathology is cancer. The term “cancer” as used herein is defined as disease characterized by the rapid and uncontrolled growth of aberrant cells. Cancer cells can spread locally or through the bloodstream and lymphatic system to other parts of the body.

The cancer may be a hematological malignancy, a solid tumor, a primary or a metatastizing tumor. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, Chronic Lymphocytic Leukemia (CLL), leukemia, lung cancer and the like. Additional non-limiting examples of cancers which can be treated by the method of some embodiments of the invention are provided in Tables 1, 15-19 above.

Cancers that may be treated include tumors that are not vascularized, or not yet substantially vascularized, as well as vascularized tumors. The cancers may comprise non-solid tumors (such as hematological tumors, for example, leukemias and lymphomas) or may comprise solid tumors. Types of cancers to be treated with the CARs of the invention include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included.

Hematologic cancers are cancers of the blood or bone marrow. Examples of hematological (or hematogenous) cancers include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia. Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases).

According to some embodiments of the invention, the pathology is a solid tumor.

According to some embodiments of the invention, the medicament resultant of the method of some embodiments of the invention has an anti-tumor effect.

The term “anti-tumor effect” as used herein, refers to a biological effect which can be manifested by a decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of the medicament of the invention in prevention of the occurrence of tumor in the first place.

According to some embodiments of the invention, the pathology is a viral infection.

Non-limiting examples of viral infections which can be treated by the medicament of some embodiments of the invention are described in Table 2 above.

According to some embodiments of the invention, the pathology is an autoimmune disease.

Non-limiting examples of autoimmune diseases which can be treated by the method and medicament of some embodiments of the invention include Addision's disease, alopecia greata, ankylosing spondylitis, autoimmune hepatitis, autoimmune parotitis, Celiac (Coeliac), Crohn's disease, diabetes (Type 1), dystrophic epidermolysis bullosa, epididymitis, glomerulonephritis, Graves' disease, Guillain-Barr syndrome, Hashimoto's disease, hemolytic anemia, systemic lupus erythematosus, multiple sclerosis, myasthenia gravis, pemphigus vulgaris, psoriasis, rheumatic fever, rheumatoid arthritis, sarcoidosis, scleroderma, Sjogren's syndrome, spondyloarthropathies, thyroiditis, vasculitis, vitiligo, myxedema, pernicious anemia, ulcerative colitis, stroke, among others.

The CAR-modified T cells of the invention may also serve as a type of vaccine for ex vivo immunization and/or in vivo therapy in a mammal. Preferably, the mammal is a human.

With respect to ex vivo immunization, of least one of the following occurs in vitro prior to administering the cell into a mammal: i) expansion of the cells, ii) introducing a nucleic acid encoding a CAR to the cells, and/or iii) cryopreservation of the cells.

Ex vivo procedures are well known in the art. Briefly, cells are isolated from a mammal (preferably a human) and genetically modified (i.e., transduced or transfected in vitro) with a vector expressing the CAR molecule of some embodiments of the invention. The CAR-modified cell can be administered to a mammalian recipient to provide a therapeutic benefit. The mammalian recipient may be a human and the CAR-modified cell can be autologous with respect to the recipient. Alternatively, the cells can be allogeneic, syngeneic or xenogeneic with respect to the recipient.

The procedure for ex vivo expansion of hematopoietic stem and progenitor cells is described in U.S. Pat. No. 5,199,942, incorporated herein by reference, can be applied to the cells of the present invention. Other suitable methods are known in the art, therefore the present invention is not limited to any particular method of ex vivo expansion of the cells. Briefly, ex vivo culture and expansion of T cells comprises: (1) collecting CD34+ hematopoietic stem and progenitor cells from a mammal from peripheral blood harvest or bone marrow explants; and (2) expanding such cells ex vivo. In addition to the cellular growth factors described in U.S. Pat. No. 5,199,942, other factors such as flt3-L, IL-1, IL-3 and c-kit ligand, can be used for culturing and expansion of the cells.

In addition to using a cell-based vaccine in terms of ex vivo immunization, the present invention also provides compositions and methods for in vivo immunization to elicit an immune response directed against an antigen in a patient.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Experimental Methods

### Media, Cells, Antibodies, Tetramer and Peptides

Unless otherwise stated, all culture medium was RPMI 1640 supplemented with 10% heat inactivated fetal calf serum (FCS), 1% penicillin and streptomycin, and 1% L-glutamine. Cell lines used were: Jurkat 76 cell line; TAP-deficient-HLA-A2⁺ T2 cell line that can be efficiently loaded with exogenous peptides; 501A and Skmel5 melanoma cell lines; Loucy and BV-173 ALL cell line; DG75 lymphoma cell line; MDA-MB-231 human breast carcinoma; SW620 and colo-205 human colon cancer; Panc-1 human pancreatic carcinoma; A431 epidermoid carcinoma cell line.

The cell lines UMUC3 human bladder transitional cell carcinoma and Fibroblast, were cultured in DMEM (Dulbecco's Modified Eagle Medium) supplemented with 10% heat inactivated FCS, 1% penicillin and streptomycin, and 1% L-glutamine.

Caco-2 human colon cancer cell line was cultured in DMEM supplemented with 20% heat inactivated FCS, 1% penicillin and streptomycin, and 1% L-glutamine. PBMCs were obtained from volunteer donors from the National Blood Service, Colindalea, London, UK.

Flow cytometry antibodies (Abs) were: anti-human PE (Jackson Immunoresearch), CD3 PerCp, CD8 APC, CD8 APC-Cy7, IFN-γ-APC, IL-2 PE (Bactlab Diagnostic), and CD107a eFluor660. PE-labeled HLA-A2/WT_{Db126} tetramers were obtained from Beckman Coulter.

The peptides used for this study were: pWT1_{Db126} (RMFPNAPYL, SEQ ID NO:1), pWT1₂₃₅ (CMTWNQMNL, SEQ ID NO:7), gp100: G2-209-2M (IMDQVPFSV, SEQ ID NO:12) and gp100-280 (YLEPGPVTA, SEQ ID NO:13), HIV: Gag (SLYNYVATL, SEQ ID NO:14), and MDM2 (LLGDLFGV, SEQ ID NO:15).

Production of Biotinylated Single-Chain MHC-Peptide Complexes—

Single-chain MHC (scMHC)-peptide complexes were produced by in-vitro refolding of inclusion bodies produced in E. coli upon IPTG induction, as previously described (33). Briefly, a scMHC containing the β2-microglobulin and the extracellular domains of the HLA-A2 gene connected to each other by a flexible linker was engineered to include the BirA recognition sequence for site specific biotinylation at the carboxyl-terminus. In-vitro refolding was performed in the presence of peptides as described. Correctly folded MHC-peptide complexes were isolated and purified by anion exchange Q-sepharose chromatography (Pharmacia), followed by site-specific biotinylation using the BirA enzyme (Avidity).

Selection of Phage Abs on Biotinylated Complexes—

Selection of phage antibodies (Abs) on biotinylated complexes was performed as previously described (27). Briefly, a large human Fab library containing 3.7×10¹⁰ different Fab clones was used for the selection. Phages were first pre-incubated with streptavidin-coated paramagnetic beads (200 μl; Dynal) to deplete the streptavidin binders. The remaining phages were subsequently used for panning with decreasing amounts of biotinylated scMHC-peptide complexes. The streptavidin-depleted library was incubated in solution with soluble biotinylated scHLA-A2-WT1 complexes (500 nM for the first round, and 100 nM for the subsequent rounds) were added to the mixture and incubated for 30 minutes at room temperature. Streptavidin-coated magnetic beads (200 μl for the first round, and 100 μl for the subsequent rounds) were added to the mixture and incubated for 10-15 minutes at room temperature. The beads were washed extensively 12 times with PBS/0.1% Tween 20 with an additional 2 washes with PBS. Bound phages were eluted with triethylamine (100 mM, 5 minutes at room temperature), followed by neutralization with Tris-Hcl (1 M, pH 7.4), and used to infect E. coli TG1 cells (OD₆₀₀=0.5) for 30 minutes at 37° C.

Expression and Purification of Soluble Recombinant Fab Abs—

Fab Abs were expressed and purified, as previously described (27). E. coli BL21 cells were grown to OD₆₀₀=0.8-1.0 and induced to express the recombinant Fab Ab by the addition of IPTG for 3-4 hours at 30° C. Periplasmic content was released using the B-PER solution (Pierce), which was applied onto a pre-washed TALON column (Clontech). Bound Fabs were eluted using 0.5 ml of 100 mM imidazole in PBS. The eluted Fabs were dialyzed twice against PBS (overnight, 4° C.) to remove residual imidazole.

Surface Plasmon Resonance (SPR)—

Kinetic studies for affinity measurements of the F2 and F3 Fabs to HLA-A2/WT1_{Db126} complexes were performed on a ProteOn XPR36 Protein Interaction Array System (Bio-Rad Laboratories, Hercules) as described before (34).

Antigen Density Quantification—

The number of specific peptide-MHC complexes on the surface of tumor cell lines was determined as previously described (35). Briefly, specific binding of F3 Fab to HLA-A2/WT1_{Db126} complexes was detected using PE-labeled anti-k L chain mAb. To transform the florescent signal obtained by flow cytometer into the number of HLA-A2/WT1_{Db126} sites, the present inventors used the QuantiBRITE PE kit (BD Biosciences) according to manufacturer's instructions.

Generation of TCR-Like CAR Retroviral Constructs—

scFv DNA of the TCR like Fabs F2 and F3 were generated by connecting the carboxyl-terminus of the V_{L} region and the amino-terminus of the V_{H} region by a peptide linker. For the chimeric receptor construct, the F2 and F3 scFvs were connected via the carboxyl-terminus of the V_{H} region to a CD28− FcγRI γ chain construct (36). The TCR-like chimeric receptor DNA constructs were cloned into retroviral pBullet vector followed by IRES and the GFP gene (37).

Transduction of Retroviral TCR Constructs into Jurkat Cells and Primary T Cells—

A total of 2×10⁶ Phoenix amphotropic packaging cells were cultured in 10-cm culture plated for 24 hours at 37° C. with 5% CO₂. The cells were transfected with the vector constructs and pCL-ampho using calcium phosphate precipitation (Invitrogen Life Technologies). After culturing for 24 hours and replacement of medium, the viral supernatant was harvested. 24 hours before retroviral transduction, Jurkat cells were split and T cells were enriched using Ficoll and RosetteSep Human T cell enrichment kit followed by and activation for 48 hours using 24 wells non-treated plates coated with anti-CD3 Ab OKT3 at 1 μg/ml and anti-CD28 at 5 μg/ml with addition of and IL-2 (600 U/ml; Chiron). For retroviral transductions, retronectin-coated (Takara) 24-well plates were seeded with cells at 1×10⁶ per well in 1 ml, cultured for 30 minutes, and then transduced with 1 ml of the constructs viral supernatant. For PBMCs (T cells), the transductions were conducted in culture medium supplemented with IL-2 at 600 U/ml. After 24 hours at 37° C. with 5% CO₂, the culture medium for Jurkat cells was replaced; for PBMCs (T cells) the replaced medium was supplemented with IL-2 at 100 U/ml. After additional 48 hours culture period, flow cytometry analysis was performed by a LSR II flow cytometer (BD Biosciences).

Intracellular IFN-γ and IL-2 Detection Assays—

Transduced T cells and T2 cells loaded with WT1_{Db126} or WT1₂₃₅ were added at 2×10⁵/well in 200 μl of culture medium containing brefeldin A (Sigma-Aldrich) at 1 μg/ml. After 16 hours at 37° C. with 5% CO₂ staining of the cells for surface CD8 was performed, followed by fixation, permeabilization, and staining for intracellular IFN-γ and IL-2 (Fix & Perm kit; Caltag). Cells were then washed and analyzed by a LSR II flow cytometer (BD Biosciences).

IFN-γ Secretion Assays—

Transduced T cells were stimulated with irradiated T2 cells (at 1:1 ratio) loaded with the WT1₁₂₆ or Gp100-280 peptide. The assay was conducted in triplicates in 200 μl medium. After 18 hours of incubation at 37° C. with 5% CO₂, the supernatant was harvested and tested for secreted IFN-γ using a human ELISA kit (BD Biosciences).

Enrichment and Depletion of Specific Subpopulations—

T-cell subpopulation depletion was obtained by incubating the T cell population with anti-CD4 or anti-CD8, for 20 minutes. After wash with PBS 0.1% BSA, cells were mixed with anti-mouse

IgG-coated magnetic beads (Dynal, invitrogen) for additional 30 minutes followed by magnetic depletion for 5 minutes. The negative fraction was then washed three times with PBS 0.1% BSA and was incubated for 24 hours recovery in 37° C. Flow cytometry analysis of purified subpopulations revealed purify above 90%.

Cytotoxic Assays—

The EBV-transformed B cell line T2 was labeled with [³⁵S] methionine for 18 hours at 37° C. with 5% CO₂. The cells were washed 3 times and loaded with the various concentrations of WT1₁₂₆ or Gp100-280 peptide for 2 hours at 37° C. with 5% CO₂. After incubation, the cells were washed of excess peptide. For different E:T ratios, peptide-loaded [³⁵S]-labeled T2 cells were added to 2-fold dilutions of transduced T cells. For the peptide titration assays, transduced cells and peptide-loaded [³⁵S]-labeled T2 cells were co-cultured at a ratio of 5:1 (E:T). After an incubation period of 4 hours at 37° C. with 5% CO₂, 25 μl of supernatant were harvested, diluted with 200 μl of scintillation fluid, and counted using a β counter (BD Biosciences, San Jose, Calif.). The percentage of specific lysis was calculated as ([experimental [³⁵S]-release−spontaneous [³⁵S]-release]/[maximum [³⁵S]-release−spontaneous [³⁵S]-release])×100, with spontaneous release being the [³⁵S]-methionine released from target cells in the absence of effector cells and maximum release being the [³⁵S]-methionine released from target cells lysed with 0.05 M NaOH. The percentage of maximal lysis was determined as the highest cytotoxic activity of target cells. In the cytotoxic assays of the CD4, CD8 enriched T cells, the amount of cells were used was normalized in respective to the highest frequency of specific transduced cells determined by tetramer or Vβ Ab staining.

Degranulation Assay—

Transduced T cells were cocultured with T2 cells (at 1:1 ratio) loaded with the WT1₁₂₆ or Gp100-280 peptide in 200 μl medium. After 4 hours of incubation at 37° C. with 5% CO₂, the cells were stained with anti-CD8 PerCP and anti-CD107a Abs for 30 minutes. The cells were washed, resuspended in PBS 0.1% BSA and the expression of CD107a on CD8⁺ transduced T cell was determined by flow cytometry (FACSCalibur, Becton Dickinson).

### Example 1

### Isolation and Characterization of TCR-Like Antibodies with Specificity to HLA-A2-WT1_{Db126} Complexes Experimental Results

Recombinant TCR-like Fab antibodies recognizing HLA-A2-WT1_{Db126} complexes were isolated by screening a large naive phage Fab library as previously described (27, 28). For panning the present inventors used recombinant single-chain HLA-A2-WT1_{Db126} complexes expressed in E. coli as insoluble inclusion bodies. Subsequently, HLA-A2-WT1_{Db126} complexes were refolded and purified using established redox-shuffling strategies (28). The phage display screening was analyzed using differential binding to specific HLA-A2-WT1_{Db126} versus control complexes and identified specific clones. Of 96 phage clones tested, 13 exhibited specific binding to recombinant HLA-A2-WT1_{Db126} complexes, compared to control complexes displaying irrelevant peptides (FIG. 1A).

Based on specific recognition to HLA-A2-WT1_{Db126} complexes, two clones, F2 and F3, were selected for further characterization and tested for their ability to bind the HLA-A2-WT1_{Db126} complexes in their native form. To this end, the present inventors used the TAP-deficient HLA-A2 positive T2 cells which were loaded with the WT1_{Db126} RMFPNAPYL (SEQ ID NO:1) peptide or control peptides. As shown in FIG. 1B, flow cytometry assays using the anti-HLA-A2-WT1_{Db126}-specific soluble purified Fabs, F2 or F3, exhibited specific binding to WT1_{Db126}-loaded APCs but not to APCs loaded with control peptides. The binding affinity of the HLA-A2/WT1_{Db126}-specific Fab TCR-like antibodies was determined by SPR using soluble HLA-A2/WT1_{Db126} complexes on chip-immobilized Fabs coupled through an anti-human Fab, and revealed an apparent affinity of 400 nM and 30 nM for F2 and F3, respectively (FIG. 1C).

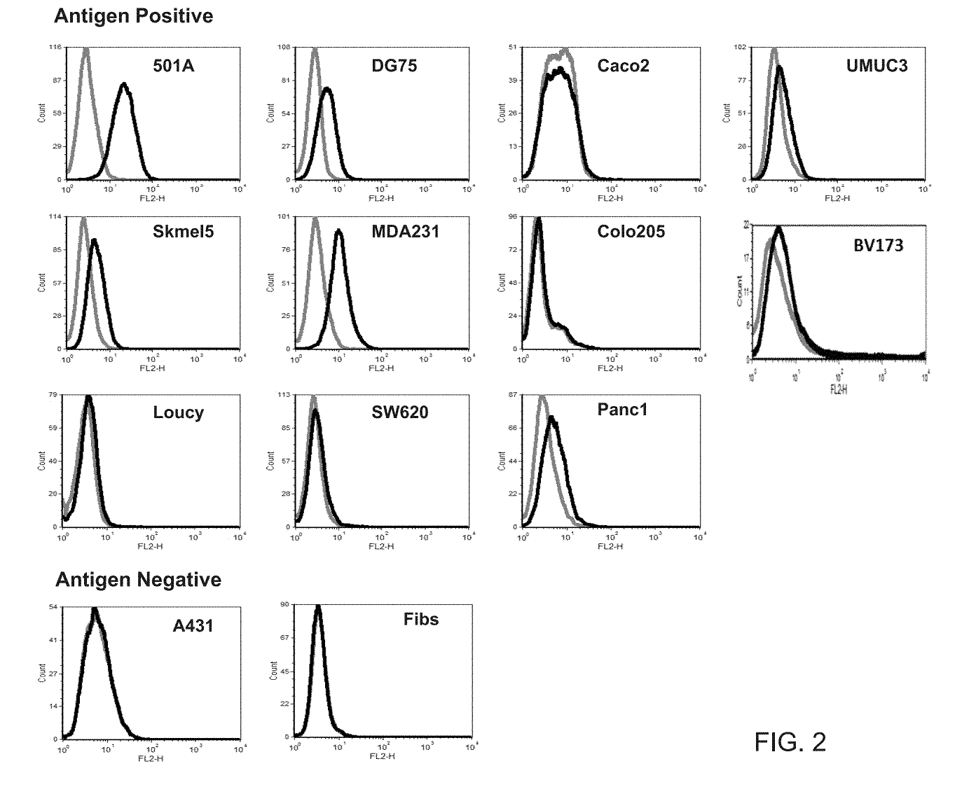

To determine whether the WT1-specific TCR-like antibodies recognize the authentic endogenously-derived HLA-A2-WT1_{Db126} complexes on the surface of target cells, the present inventors tested binding using a large panel of tumor cell lines. As shown in FIG. 2, the F2 Fab recognized HLA-A2-WT1_{Db126} positive cells. No reactivity was observed with WT1-negative fibroblasts cells or HLA-A2-negative A431 cells. Similar data was observed with the F3 Fab (data not shown). The endogenous expression of WT1 transcript in these tumor cell lines was determined by mRNA analysis. The present inventors have further quantified the number of HLA-A2-WT1_{Db126} complexes expressed on the surface of tumor cell lines using the F3 Fab and the total number of HLA-A2 molecules expressed on the cell surface using the anti-HLA-A2 antibody BB7.2. Overall, these data indicate that Fabs F2 and F3 exhibit properties of TCR-like antibodies, they bind with HLA-A2 restriction and WT1_{Db126} peptide specificity to peptide-loaded APCs as well as to tumor target cells that present the antigen in an HLA-restricted manner and with high affinity (30 nM and 400 nM, respectively) compared to native TCRs.

### Example 2

### Expression of TCR Like ABs as Chimeric Antigen Receptors (CARs)

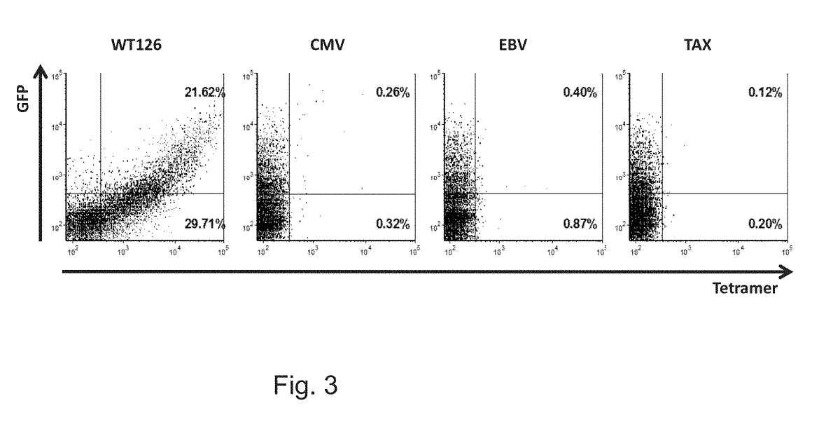

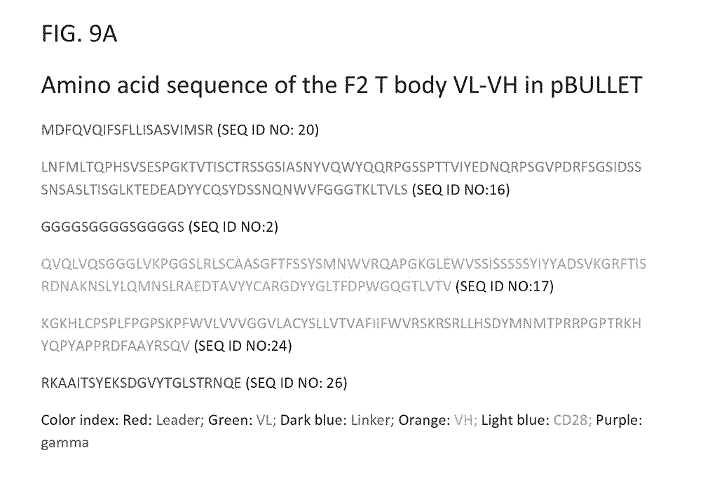

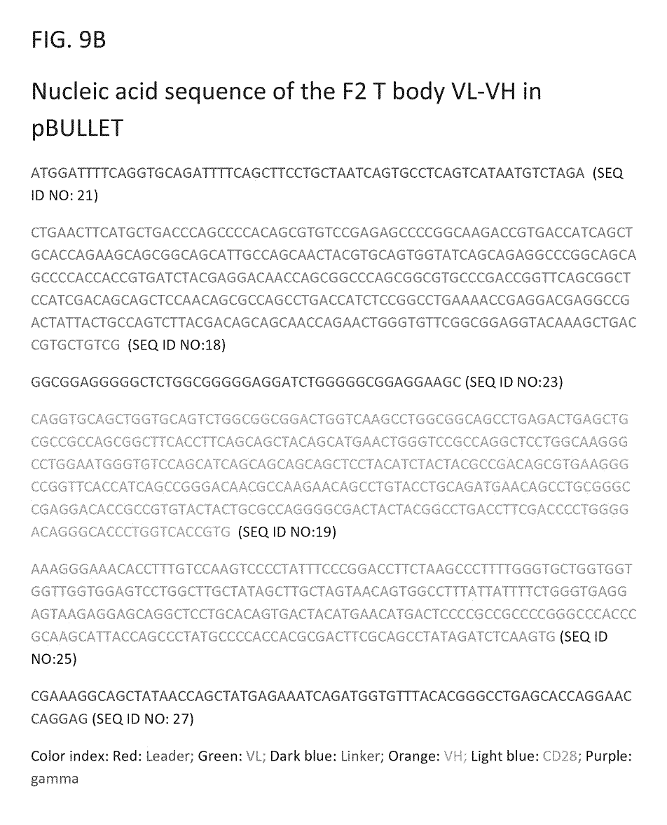

Previous CAR studies revealed that employing single chain CARs is an efficient strategy to redirect T cells using Ab fragments, as it requires only a single retroviral transduction step (38). Therefore, the present inventors generated scFv forms of the F2 and F3 TCR-like Fab Abs by fusing the carboxyl-terminus of the VL domain to the amino-terminus of the VH domain using a flexible (Gly4Ser)3 peptide linker (SEQ ID NO:2). The complete CAR molecule was composed of the F2 or F3 scFv connected via the carboxyl-terminus of the VH domain to a CD28-γ chain construct. FIGS. 9A-B and 10A-B provide the sequences of the F2 and F3 CARs, respectively. This CAR construct was introduced into a retroviral vector (pBULLET) which also harbors a GFP reporter gene to visualize cells transduced with the vector (37). FIG. 3 represents an analysis of TCR-negative Jurkat-76 cells transduced with the CAR retroviral vectors encoding the F2 or the F3 chimeric receptors. The present inventors examined the staining of GFP-positive transduced cells with WT1 and control tetramers and found that GFP-positive cells representing transduced cells were co-stained with high frequency of >20% with HLA-A2-WT1_{Db126}-specific tetramers but not with tetramers displaying irrelevant peptides. Data are shown for the F2 CAR and similar results were obtained for F3.

Next, the present inventors tested the transduction efficiency in human primary T cells. F2 (400 nM) and F3 (30 nM) TCR like CARs were retro-virally transduced into HLA-A2 positive human T cells. FIG. 4A shows that CD8⁺ human T cells were efficiently transduced to express significant levels of the F2 TCR-like CAR as evident by staining of transduced cells with HLA-A2-WT1_{Db126} tetramers. Human CD8⁺ T cells transduced with the F3 TCR-like CAR expressed high level of CAR as revealed by tetramer staining; however, the viability of the transduced cells was very low. As shown in FIG. 4A, the remaining fraction of viable CD8+ transduced T cells expressed the receptor at high levels (>60%) as determined by tetramer staining. Retroviral transduction of the 30 nM TCR-like F3 CAR into HLA-A2 negative cells yielded cell surface expression comparable to that observed for F2 (FIG. 4B), but with much higher viability rate (most transduced cells were viable). These results suggest that the elevated affinity of the 30 nM TCR-like CAR, F3, in combination with the high avidity of the receptor present on the T cell surface, may lead to some loss of specificity and consequently to decreased cell survival. The F2 TCR-like CAR, having moderate affinity of 400 nM, exhibited expression properties and characteristics that are more suitable for the comparison between T cells redirected by either CAR based on TCR-like Ab fragments or by a cloned αβTCR.

### Example 3

### Efficiency of Transduction of T Cells Redirected by Either ABTCR or TCR-Like Antibody-Based CAR

To compare the specificity and function of T cells retargeting by either αβTCRs or TCR-like antibody-based CARs, the present inventors used an αβTCR specific to the HLA-A2-WT1_{Db126} epitope which has been previously isolated and characterized by Prof. Stauss's group (the University College of London) and was inserted into the retroviral vector MP71 (7). The present inventors studied the transduction efficiency of the WT1_{Db126} αβTCR (also referred to as “αβTCR”) versus that of the F2 TCR like CAR, using retroviral transductions into primary human T cells. FIG. 5 demonstrates efficient transductions of HLA-A2+ T cells transduced with either the αβTCR construct (as determined by V132.1 or HLA-A2-WT1_{Db126} tetramer staining) or the F2 TCR like CAR construct (as determined by HLA-A2-WT1_{Db126} tetramer staining). The αβTCR construct has the addition of an internal disulphide bond to minimize the mispairing of the exogenous TCR α and β chains with the endogenous TCR chains (39). However, HLA-A2-WT1_{Db126} tetramer staining of the αβTCR transduced T cells pointed to low levels of expression. This observation may have been due to either a low expression of functional αβTCR which will not facilitate tetramer binding or due to TCR mispairing.

### Example 4

### Activation of αβTCR or TCR-Like Ab CAR-Transduced T Cells

As a first step for functional evaluation and comparison of T cells transduced with αβTCR or TCR-like Ab CAR the present inventors tested their ability to undergo proper activation. αβTCR or F2-TCR-like Ab CAR-transduced T cells were stimulated with the human TAP-deficient T2 cells loaded with either the specific peptide (WT1_{Db126}) or an irrelevant peptide (WT1₂₃₅) as a control. Following overnight stimulation, the cells were stained for CD8 and for intracellular IFN-γ and IL-2. As shown in FIG. 6A, CAR-transduced T cells and αβTCR transduced T cells were activated in an antigen-specific manner as indicated by the comparable number of transduced T cells that were intracellularly stained with anti-IL-2 and/or IFN-γ.

In order to test the avidity (also referred to as “functional avidity”) of the transduced T cells, the present inventors examined the dose dependency and peptide specificity of IFN-γ secretion by transduced T cells in an ELISA assay. As shown in FIG. 6B, T cells transduced with the WT1 αβTCR construct were more sensitive to lower peptide concentrations as compared to T cells transduced with the F2 TCR-like Ab CAR. T cells expressing the WT1 αβTCR secreted higher levels of IFN-γ in response to T2 cells loaded with peptide concentration ranging from 0.3 to 100 μM, whereas T cells expressing the F2 TCR-like CAR secreted considerably reduced levels of IFN-γ at peptide concentrations starting as low as 0.30 μM. At 1 μM T cell transduced with native TCR (i.e., the WT1 αβTCR construct) secreted significant levels of IFN-γ while the TCRL-transduced T cells did not secret at all. Notably, at 10 μM, the WT1 αβTCR-transduced T cells exerted high and close to maximal IFN-γ secretion, whereas the TCR-like Ab CAR-transduced T cells showed ˜50% less secretion. From these results it can be determined that the avidity of the T cells transduced with the CAR of WT1 αβTCR is between 3-10 μM, e.g., about 5 μM, and the avidity of the T cells transduced with the CAR of F2 TCRL is about 10 μM.

In addition to measuring IFN-γ secretion the present inventors assessed the expression of CD107a, a marker of CD8+ T-cell degranulation following stimulation, on the surface of the transduced T cells (FIG. 6C). Confirming the cytokine secretion assays, the present inventors observed significant differences in the expression level of CD107a as a function of WT1_{Db126} peptide concentration. The F2 TCR-like Ab transduced-T cells expressed significant lower levels of CD107a compared with the TCR-transduced T cells which indicates a much lower degranulation and stimulation levels. At low peptide doses, of 1 and 3 μM, major 3-10-fold difference in CD107a expression was observed (FIG. 6C). These results indicate major differences in antigen sensitivity of the αβTCR versus TCR-like Ab CARs and consequently in T cell stimulation and cytokine secretion.

### Example 5

### Cytolytic Activity of T Cells Transduced with TCR-Like Ab Versus of TCR CARs

Next, the present inventors compared the F2 TCR-like Ab CAR to the engineered αβTCR for their cytolytic activity towards ³⁵S-methionine-labelled T2 cells loaded with either a relevant (WT1_{Db126}) or irrelevant control peptide. As shown in FIG. 7A, T cells transduced with the αβTCR showed greater specific cytolytic activity than T cells transduced with the F2 TCR-like Ab CAR. Both transduced T cells maintained their specificity towards the WT1 peptide as their background cytotoxic activity towards a control non-specific peptide was similar and low (FIG. 7A). The transduced T cells exhibited also low accepted background cytotoxicity to T2 APCs without any loaded peptide (FIG. 7B).

In order to examine the antigen sensitivity of both receptors, the present inventors performed a killing assay using ³⁵S-methionine-labeled T2 cells loaded with decreasing concentrations of the WT1_{Db126} specific peptide.

As shown in FIG. 7B, the sensitivity of the αβTCR-transduced T cells was indeed greater compared with the TCR-like Ab-transduced cells as the αβTCR cells were more efficient in mediating killing of WT1_{Db126} loaded T2 cells at low peptide concentrations. As observed before, this was most significant at the lower peptide doses of 1-3 μM, with 4-20-fold difference.

The present inventors also tested the killing sensitivity of the two types of transduced T cells on tumor cells that express the native HLA-A2/WT1_{Db126} complex. For this purpose the present inventors used HLA-A2+ WT1 ³⁵S-methionine-labeled antigen positive 501A and MDA231 cells and HLA-A2−/WT1+ A431 cells as control. As shown in FIG. 7C, a marked and significant difference was observed in the killing sensitivity of the αβTCR transduced T cells as compared to the TCR-like Ab CAR T cells at various effector to target (E:T) ratios. While the αβTCR transduced T cells exhibited E:T-dependent killing of 501A and MDA231 tumor target cells, the TCR-like Ab-transduced cells showed only very marginal minor killing activity on these cells (FIG. 7C). These results correspond to the aforementioned observation that T cells transduced with the αβTCR exhibited higher antigen sensitivity compared to the TCR-like Ab transduced cells, and consequently results in more efficient cytolytic activity toward tumor cells.

Next, the present inventors attempted to further investigate the differences between CAR and TCR-transduced T cells concerning the relative contribution of the CD8 and CD4 subpopulations to overall T cell-mediated cytotoxic activity. Thus, the present inventors transduced primary T cells with the F2 TCRL CAR or the αβTCR CAR constructs and derived the CD4+ and CD8+ T-cell subpopulations by depletion with anti-CD8 or anti-CD4, respectively. Flow cytometry analysis of purified subpopulations revealed purify of above 95% (data not shown). Using the isolated purified CD4+ and CD8+ subpopulations the present inventors tested their sensitivity and function in killing assays in which peptide-pulsed ³⁵S-methionine-labeled T2 cells were used as targets (FIG. 8). The results show that the difference in sensitivity and function of αβTCR CAR vs. F2 TCRL CAR-transduced T cells was maintained also for the purified subpopulation of CD8+ T cells when peptide sensitivity (FIG. 8A) and E:T ratio (FIG. 8B) were tested. TCR-transduced CD8+ T cells were more active compared to CAR-transuded cells similarly with what was observed with the intact whole T cell population. The present inventors also observed that most killing activity was mediated by CD8+ T cells (comparing data in FIGS. 8A and B to FIGS. 8C and D) and CD4+ cells exhibited minor killing activity of up to 30% killing compared to very efficient killing (up to 100% of relative maximal killing) with CD8+ T cells. Interestingly, purified CD4+ cells of F2 TCRL CAR-transduced T cells was somewhat more efficient compared with the αβTCR CAR transduced cells in most of the peptide doses tested (FIG. 8C) and at high E:T ratios (FIG. 8D). These results demonstrate that the F2 TCRL CAR construct confers the T cell a TCR-like specificity which is not CD8-dependent.

Overall, the data with total un-separated T cells transduced with αβTCR CAR or the F2 TCRL CAR as well as the CD8+ and CD4+ subpopulations further support the findings that an upper affinity threshold for TCR-based recognition is required to mediate effective and optimal functional activity in killing of target cells. When these principals are applied to the functional outcomes of engineered T cells, the rational design of TCRs and TCR-based constructs may need to be optimized to a given affinity threshold in order to achieve optimal T cell function.

Analysis and Discussion

Engineered T cells constitute a powerful tool to redirect T cells to a desired target for immunotherapy and are being tested in clinical trials (1, 3, 5, 7, 9, 10, 15, 18, 40, 41). Recent advances in TCR/antibody engineering led to 2 promising approaches in adoptive cell transfer for cancer therapy. One is the ability to modify TCR sequences to increase their affinity for cognate tumor antigen epitopes (42-44). For example, various strategies such as phage-display of TCR libraries have led to the generation of TCR variants with supra-physiological binding affinity [up to pico molar (pM)] towards epitopes derived from NY-ESO-1 or HTLV-1 (43). The second major advance was the ability to generate high affinity TCR-like antibodies that bind the HLA-peptide complex with high affinity and specificity in the low nM affinity range (27-30). However, in contrast to native TCRs that require further affinity engineering and sequence manipulation, these are native antibodies made by variable domain recombination using antibody phage display libraries or native antibody germ-line sequences made by hybridoma approaches from immunized mice.

This study has aimed to directly compare, for the first time, engineered T cells that carry 2 forms of HLA-peptide complex-based recognition moieties: a naturally cloned αβTCR versus a corresponding recombinant TCR-like antibody-based CAR, both recognizing the same antigenic epitope derived from TAA WT1. The ability of engineered T cells based on the cloned αβTCR strategy to recognize and specifically kill tumor cells expressing the desired target has been previously demonstrated (6-11). Here, the present inventors investigated the transduction efficiency of the 2 constructs into primary human T cells and compared the biological functions of the transduced cells.

To generate a chimeric antigen receptor based on TCR-like Ab fragments, 2 TCR-like Fab antibodies were isolated that target the HLA-A2-WT1_{Db126} complex with affinities of 400 nM and 30 nM. These TCR-like Fabs exhibited high specificity by their ability to discriminate between HLA-A2 complexes displaying the specific WT1_{Db126} peptide and HLA-A2 displaying irrelevant control peptides. Furthermore, the WT1-specific TCR-like antibodies recognized tumor cells that displayed the naturally processed and presented WT1 epitope in the context of HLA-A2. Recognition was peptide- and HLA-specific which merits further evaluation of these TCR-like antibodies for antibody-based tumor targeting using antibody arming strategies such as antibody-drug conjugates (ADC) (45) or T-cell engagement approaches through the use of antibody bi-specific constructs with anti-CD3 or anti-CD16 (46).

Using these TCR like Fabs in the context of CARs, as designed by Eshhar et al (38), revealed that retroviral transduction of the high affinity F3 TCR-like Ab (30 nM) into HLA-A2 positive cells induced massive cell death of transduced cells, with a small fraction of viable cells expressing the receptor. Without being limited by any theory, the present inventors hypothesize that this CAR lost its specificity and mediated non-specific killing of neighbor lymphocytes by recognizing HLA-A2 complexes regardless of their presented peptides. It is likely that the avidity of the chimeric receptors is increased because they are expressed on the cell surface at relatively high levels and that this high avidity, combined with the high affinity of this receptor, affects the receptor ability to maintain its specificity, leading to a leakage in its discrimination ability (i.e., cross reactivity with HLA-A2 complexes presenting irrelevant peptides). This notion is supported by transduction experiments in HLA-A2 negative T cells in which most of the cells were viable and a fraction expressed the CAR. In this case, due to absence of HLA-A2 complexes on the surface of the cells, no cytotoxic activity was observed by transduced T cells which expressed the 30 nM F3 TCR-like CAR. Additional supporting evidence includes the previous finding by the present inventors which demonstrated that HLA-A2-specific TCR-like antibodies may cross-react with HLA-A2 molecules presenting other HLA-A2-restricted peptides but not with other HLA alleles (unpublished data). Studies with affinity-matured native TCRs demonstrated that although some of the identified affinity enhanced variants showed superior T-cell function, the increase in affinity oftentimes led to loss of target cell specificity (44, 47).

The data presented herein are supported by a recent study assessing the relationship of TCR affinity, TCR-peptide-MHC binding parameters and T cell function. This study tested a panel of sequence-optimized HLA-A2/NY-ESO-1 specific TCR variants with affinities lying within physiological boundaries to preserve antigenic specificity and avoid cross-reactivity, as well as 2 variants with a very high and a low-affinity. Primary human CD8 T cells transduced with these TCRs demonstrated robust correlations between binding measurements of TCR affinity and avidity and the biological response of the T cells, such as TCR cell-surface clustering, intracellular signaling, proliferation, and target cell lysis (49). Strikingly, and as observed by the present inventors herein, above a threshold of TCR-peptide-MHC affinity (K_{D}<□5 μM), T cell function could not be further enhanced, revealing a plateau of maximal T cell function, compatible with the notion that multiple TCRs with slightly different affinities participate equally (co-dominantly) in immune responses. Similar to the present study, TCRs displaying affinity above the defined threshold exhibited non-specific recognition by the T cell receptor. The observation with the TCR-like antibody-based CAR is consistent with this study and indicates a functional threshold for optimal affinity of engineered TCR or antibody-based CARs. The work performed on the NY-ESO-1 αβTCR CARs defined the upper affinity limit of TCR for specific antigen recognition as approximately 280-450 nM (44, 49).

The affinity of the F2 TCR-like CAR as soluble monovalent antibody fragment was found to be 400 nM, at the uppermost border of this limit. The data presented herein for the WT1 TCR-like antibody-based CAR demonstrate that specificity for the WT1 peptide epitope was maintained. However, in other studies that examined high affinity TCR-based CARs alterations in peptide specificity were observed (49). Thus, the possibility that high affinity TCR or TCR-like antibody based CARs may still bind to other self-peptide complexes at lower affinity and densities compared to the specific peptide cannot be excluded. Each CAR specificity should be examined carefully as these properties are crucial for clinical applications.

Based on the transduction data of the F3 (30 nM) and F2 (400 nM) TCR-like CARs, the present inventors have further compared the F2 TCR-like antibody CAR with the cloned αβTCR receptors. Each one of the receptors was introduced into human primary T cells by retroviral transduction. The expression of functional αβTCR or TCR-like CAR on the transduced cells was measured by specific WT1 tetramer binding and revealed that expression of functional TCR-like CAR was higher compared to the TCR (38% vs. 12%). Despite the difference these data were reproducible and consistent enabling the comparison of their ability to properly activate and mediate the biological functions of the transduced T cells. The present inventors found that both constructs were capable of properly activating the engineered T cells as indicated by intracellular cytokine expression; however, cytokine secretion assays revealed that the αβTCR construct was more sensitive to peptide target concentrations. These results were surprising as the 400 nM F2 TCR-like CAR exhibited higher affinity than the native TCR, which appears to have an affinity of 1 μM. The higher affinity receptor was expected to bind to lower concentrations of antigen than the native TCR (i.e., higher levels of cytokine secretion at lower target peptide concentrations). Moreover, as indicated above, tetramer staining revealed that the TCR-like CAR was expressed better on the cell surface compared to the αβTCR which further strengthen this observation. Without being bound by any theory, these results may be explained by impaired T cell activation caused by the high affinity TCR-like antibody CAR with agreement with the serial triggering model. This model suggests that for efficient T-cell activation, multiple TCRs on the cell surface should sample the MHC-peptide complex, a process depending on adequately short dissociation rates of the receptor-ligand interaction (32). Therefore, the high affinity of the TCR-like-CAR may have reduced the dissociation rates leading to antigen sensitivity reduction. Similarly, a recently published study using affinity-matured TCRs demonstrated that TCRs displaying affinity above the physiological range may exhibit reduced sensitivity to their corresponding ligand (48).

The cytotoxic activity of the transduced engineered cells whether tested as intact un-separated population of cells or after separation to CD8+ and CD4+ subpopulations, supported the aforementioned data. The TCR-like antibody-based CAR transduced cells exhibited a reduced cytotoxic activity compared to the αβTCR transduced cells, which exhibited significant and specific cytotoxic activity. Both transduced T cells expressing the αβTCR or the F2 TCRL CAR exhibited specific killing of target cells expressing the specific WT1 epitope, and did not recognize cells presenting an irrelevant epitope of WT1 nor antigen negative tumor cells. Finally, CD8 is not likely to play the same role in the CAR transduced T cells as it does with the conventional TCR/CD3 complex. This also can explain why the higher affinity CAR did not operate with the sensitivity of the TCR.

Without being bound by any theory, one conclusion from this work is that high affinity TCR-like antibodies are not suitable for optimal construction of CARs designed to retarget T cells. Another important aspect of this work is the relationships between affinity and avidity when comparing T cell and antibody-based immunotherapeutic approaches. The affinity of an antibody or TCR is defined as the binding strength of a single molecule to a cognate antigen. Distinguishably, TCR binding avidity is defined as the binding strength of multiple cell surface TCRs to their respective antigen while the avidity of soluble recombinant antibodies may be monovalent or bivalent. Hence, when using a TCR-like antibody as a CAR, the natural avidity of the soluble antibody is out of context from its natural properties. Therefore, all the potential effects arising from the cellular context including cell surface receptor density, MHC co-receptors, and T-cell activation state, should also be considered when constructing high affinity TCR-like antibody-based CARs (32). The overall sensitivity of T cells to antigen density is termed ‘functional avidity’ and includes the relative affinity of the TCR-MHC-peptide interaction and the subsequent efficiency of the downstream signal transduction. Since functional avidity refers to the actual sensitivity of the cellular response to MHC-peptide antigen density, all aspects of a TCR binding to peptide-MHC (pMHC) i.e., kinetic constants, avidity and relative affinity, have direct implications for efficient T cell function. Studies have demonstrated that CTLs that exhibit high functional avidities and hence, higher sensitivity, are more effective and therefore essential for antitumor response (50). Herein, the present inventors showed that the high affinity TCR-like antibody in a monovalent or bivalent context (data with IgG not shown) maintained high specificity. However, when the avidity of this TCR-like antibody was increased to a high level, as expressed on the surface of the engineered T cells as a CAR, it had significant consequences on T cell functions. The present inventors have isolated two Fabs with high and moderate affinity towards the WT1 epitope. Perhaps the isolated Fabs bind preferentially to WT1, but still cross-react with lower affinity for other self-peptides. Other Fabs against other targets may be better in this regard and such studies should be expended to have a more complete picture on the relationships between affinity, avidity, and specificity.

In summary, this study strongly supports the notion that optimal TCRs or TCR-based constructs possess a threshold affinity beyond which no improvement in T cell functions is achieved. When TCR-based constructs or TCR-like antibody affinity is enhanced to high and supra-physiological affinities, engineered T cells carrying such CARs react with many different pMHC complexes and may change T cell functional properties leading to unresponsiveness or in some cases loss of antigen specificity, leading to dangerous cross-reactivity. Therefore, rational design of improved TCRs to generate optimal CARs may need to be optimized up to a given affinity threshold in order to achieve optimal T cell function without risking T cells to undergo unresponsiveness or cross reactivity. In this context, high affinity CARs which can be composed of TCRL antibody variable fragments, or affinity enhanced αβTCRs are less suitable and attractive than native αβTCR or TCRL antibodies with moderate affinity (e.g., having a K_{D} higher than 150 nM) for the design of CARs.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### REFERENCES

### Additional References are Cited in Text

1. Rosenberg, S. A., N. P. Restifo, J. C. Yang, R. A. Morgan, and M. E. Dudley. 2008. Adoptive cell transfer: a clinical path to effective cancer immunotherapy. Nat Rev Cancer 8: 299-308.
2. Dudley, M. E., J. R. Wunderlich, P. F. Robbins, J. C. Yang, P. Hwu, D. J. Schwartzentruber, S. L. Topalian, R. Sherry, N. P. Restifo, A. M. Hubicki, M. R. Robinson, M. Raffeld, P. Duray, C. A. Seipp, L. Rogers-Freezer, K. E. Morton, S. A. Mavroukakis, D. E. White, and S. A. Rosenberg. 2002. Cancer regression and autoimmunity in patients after clonal repopulation with antitumor lymphocytes. Science 298: 850-854.
3. Besser, M. J., R. Shapira-Frommer, A. J. Treves, D. Zippel, O. Itzhaki, L. Hershkovitz, D. Levy, A. Kubi, E. Hovav, N. Chermoshniuk, B. Shalmon, I. Hardan, R. Catane, G. Markel, S. Apter, A. Ben-Nun, I. Kuchuk, A. Shimoni, A. Nagler, and J. Schachter. 2010. Clinical responses in a phase II study using adoptive transfer of short-term cultured tumor infiltration lymphocytes in metastatic melanoma patients. Clin Cancer Res 16: 2646-2655.
4. Rosenberg, S. A., J. C. Yang, R. M. Sherry, U. S. Kammula, M. S. Hughes, G. Q. Phan, D. E. Citrin, N. P. Restifo, P. F. Robbins, J. R. Wunderlich, K. E. Morton, C. M. Laurencot, S. M. Steinberg, D. E. White, and M. E. Dudley. 2011. Durable complete responses in heavily pretreated patients with metastatic melanoma using T-cell transfer immunotherapy. Clin Cancer Res 17: 4550-4557.
5. Restifo, N. P., M. E. Dudley, and S. A. Rosenberg. 2012. Adoptive immunotherapy for cancer: harnessing the T cell response. Nat Rev Immunol 12: 269-281.
6. Cohen, C. J., Z. Zheng, R. Bray, Y. Zhao, L. A. Sherman, S. A. Rosenberg, and R. A. Morgan. 2005. Recognition of fresh human tumor by human peripheral blood lymphocytes transduced with a bicistronic retroviral vector encoding a murine anti-p53 TCR. J immunol 175: 5799-5808.
7. Xue, S. A., L. Gao, D. Hart, R. Gillmore, W. Qasim, A. Thrasher, J. Apperley, B. Engels, W. Uckert, E. Morris, and H. Stauss. 2005. Elimination of human leukemia cells in NOD/SCID mice by WT1-TCR gene-transduced human T cells. Blood 106: 3062-3067.
8. Stanislawski, T., R. H. Voss, C. Lotz, E. Sadovnikova, R. A. Willemsen, J. Kuball, T. Ruppert, R. L. Bolhuis, C. J. Melief, C. Huber, H. J. Stauss, and M. Theobald. 2001. Circumventing tolerance to a human MDM2-derived tumor antigen by TCR gene transfer. Nat immunol 2: 962-970.
9. Morgan, R. A., M. E. Dudley, J. R. Wunderlich, M. S. Hughes, J. C. Yang, R. M. Sherry, R. E. Royal, S. L. Topalian, U. S. Kammula, N. P. Restifo, Z. Zheng, A. Nahvi, C. R. de Vries, L. J. Rogers-Freezer, S. A. Mavroukakis, and S. A. Rosenberg. 2006. Cancer regression in patients after transfer of genetically engineered lymphocytes. Science 314: 126-129.
10. Johnson, L. A., R. A. Morgan, M. E. Dudley, L. Cassard, J. C. Yang, M. S. Hughes, U. S. Kammula, R. E. Royal, R. M. Sherry, J. R. Wunderlich, C. C. Lee, N. P. Restifo, S. L. Schwarz, A. P. Cogdill, R. J. Bishop, H. Kim, C. C. Brewer, S. F. Rudy, C. VanWaes, J. L. Davis, A. Mathur, R. T. Ripley, D. A. Nathan, C. M. Laurencot, and S. A. Rosenberg. 2009. Gene therapy with human and mouse T-cell receptors mediates cancer regression and targets normal tissues expressing cognate antigen. Blood 114: 535-546.
11. Robbins, P. F., R. A. Morgan, S. A. Feldman, J. C. Yang, R. M. Sherry, M. E. Dudley, J. R. Wunderlich, A. V. Nahvi, L. J. Helman, C. L. Mackall, U. S. Kammula, M. S. Hughes, N. P. Restifo, M. Raffeld, C. C. Lee, C. L. Levy, Y. F. Li, M. El-Gamil, S. L. Schwarz, C. Laurencot, and S. A. Rosenberg. 2011. Tumor regression in patients with metastatic synovial cell sarcoma and melanoma using genetically engineered lymphocytes reactive with NY-ESO-1. J Clin Oncol 29: 917-924.
12. Uttenthal, B. J., I. Chua, E. C. Morris, and H. J. Stauss. 2012. Challenges in T cell receptor gene therapy. J Gene Med 14: 386-399.
13. Gross, G., T. Waks, and Z. Eshhar. 1989. Expression of immunoglobulin-T-cell receptor chimeric molecules as functional receptors with antibody-type specificity. Proc Natl Acad Sci USA 86: 10024-10028.
14. Eshhar, Z. 2008. The T-body approach: redirecting T cells with antibody specificity. Exp Pharmacol: 329-342.
15. Kershaw, M. H., J. A. Westwood, L. L. Parker, G. Wang, Z. Eshhar, S. A. Mavroukakis, D. E. White, J. R. Wunderlich, S. Canevari, L. Rogers-Freezer, C. C. Chen, J. C. Yang, S. A. Rosenberg, and P. Hwu. 2006. A phase I study on adoptive immunotherapy using gene-modified T cells for ovarian cancer. Clin Cancer Res 12: 6106-6115.
16. Pule, M. A., B. Savoldo, G. D. Myers, C. Rossig, H. V. Russell, G. Dotti, M. H. Huls, E. Liu, A. P. Gee, Z. Mei, E. Yvon, H. L. Weiss, H. Liu, C. M. Rooney, H. E. Heslop, and M. K. Brenner. 2008. Virus-specific T cells engineered to coexpress tumor-specific receptors: persistence and antitumor activity in individuals with neuroblastoma. Nat Med 14: 1264-1270.
17. Kochenderfer, J. N., W. H. Wilson, J. E. Janik, M. E. Dudley, M. Stetler-Stevenson, S. A. Feldman, I. Maric, M. Raffeld, D. A. Nathan, B. J. Lanier, R. A. Morgan, and S. A. Rosenberg. 2010. Eradication of B-lineage cells and regression of lymphoma in a patient treated with autologous T cells genetically engineered to recognize CD19. Blood 116: 4099-4102.
18. Porter, D. L., B. L. Levine, M. Kalos, A. Bagg, and C. H. June. 2011. Chimeric antigen receptor-modified T cells in chronic lymphoid leukemia. N Engl J Med 365: 725-733.
19. Jena, B., G. Dotti, and L. J. Cooper. 2010. Redirecting T-cell specificity by introducing a tumor-specific chimeric antigen receptor. Blood 116: 1035-1044.
20. Cheever, M. A., J. P. Allison, A. S. Ferris, O. J. Finn, B. M. Hastings, T. T. Hecht, I. Mellman, S. A. Prindiville, J. L. Viner, L. M. Weiner, and L. M. Matrisian. 2009. The prioritization of cancer antigens: a national cancer institute pilot project for the acceleration of translational research. Clin Cancer Res 15: 5323-5337.
21. Inoue, K., H. Ogawa, Y. Sonoda, T. Kimura, H. Sakabe, Y. Oka, S. Miyake, H. Tamaki, Y. Oji, T. Yamagami, T. Tatekawa, T. Soma, T. Kishimoto, and H. Sugiyama. 1997. Aberrant overexpression of the Wilms tumor gene (WT1) in human leukemia. Blood 89: 1405-1412.
22. Oji, Y., H. Ogawa, H. Tamaki, Y. Oka, A. Tsuboi, E. H. Kim, T. Soma, T. Tatekawa, M. Kawakami, M. Asada, T. Kishimoto, and H. Sugiyama. 1999. Expression of the Wilms' tumor gene WT1 in solid tumors and its involvement in tumor cell growth. Jap J Cancer Res: Gann 90: 194-204.
23. Yamagami, T., H. Sugiyama, K. Inoue, H. Ogawa, T. Tatekawa, M. Hirata, T. Kudoh, T. Akiyama, A. Murakami, and T. Maekawa. 1996. Growth inhibition of human leukemic cells by WT1 (Wilms tumor gene) antisense oligodeoxynucleotides: implications for the involvement of WT1 in leukemogenesis. Blood 87: 2878-2884.
24. Oka, Y., O. A. Elisseeva, A. Tsuboi, H. Ogawa, H. Tamaki, H. Li, Y. Oji, E. H. Kim, T. Soma, M. Asada, K. Ueda, E. Maruya, H. Saji, T. Kishimoto, K. Udaka, and H. Sugiyama. 2000. Human cytotoxic T-lymphocyte responses specific for peptides of the wild-type Wilms' tumor gene (WT1) product. Immunogenetics 51: 99-107.
25. Gao, L., I. Bellantuono, A. Elsasser, S. B. Marley, M. Y. Gordon, J. M. Goldman, and H. J. Stauss. 2000. Selective elimination of leukemic CD34(+) progenitor cells by cytotoxic T lymphocytes specific for WT1. Blood 95: 2198-2203.
26. Dahan, R., and Y. Reiter. 2012. T-cell-receptor-like antibodies—generation, function and applications. Expert Rev Mol Med 14: e6.
27. Cohen, C. J., G. Denkberg, A. Lev, M. Epel, and Y. Reiter. 2003. Recombinant antibodies with MHC-restricted, peptide-specific, T-cell receptor-like specificity: new tools to study antigen presentation and TCR-peptide-MHC interactions. J Mol Recognit 16: 324-332.
28. Denkberg, G., C. J. Cohen, A. Lev, P. Chames, H. R. Hoogenboom, and Y. Reiter. 2002. Direct visualization of distinct T cell epitopes derived from a melanoma tumor-associated antigen by using human recombinant antibodies with MHC-restricted T cell receptor-like specificity. Proc Natl Acad Sci USA 99: 9421-9426.
29. Griffiths, A. D., S. C. Williams, O. Hartley, I. M. Tomlinson, P. Waterhouse, W. L. Crosby, R. E. Kontermann, P. T. Jones, N. M. Low, T. J. Allison, and et al. 1994. Isolation of high affinity human antibodies directly from large synthetic repertoires. EMBO J 13: 3245-3260.
30. Sergeeva, A., G. Alatrash, H. He, K. Ruisaard, S. Lu, J. Wygant, B. W. McIntyre, Q. Ma, D. Li, L. St John, K. Clise-Dwyer, and J. J. Molldrem. 2011. An anti-PR1/HLA-A2 T-cell receptor-like antibody mediates complement-dependent cytotoxicity against acute myeloid leukemia progenitor cells. Blood 117: 4262-4272.
31. Davis, M. M., J. J. Boniface, Z. Reich, D. Lyons, J. Hampl, B. Arden, and Y. Chien. 1998. Ligand recognition by alpha beta T cell receptors. Annu Rev Immunol 16: 523-544.
32. Stone, J. D., A. S. Chervin, and D. M. Kranz. 2009. T-cell receptor binding affinities and kinetics: impact on T-cell activity and specificity. Immunology 126: 165-176.
33. Denkberg, G., C. J. Cohen, D. Segal, A. F. Kirkin, and Y. Reiter. 2000. Recombinant human single-chain MHC-peptide complexes made from E. coli By in vitro refolding: functional single-chain MHC-peptide complexes and tetramers with tumor associated antigens. Eur J Immunol 30: 3522-3532.
34. Bronner, V., G. Denkberg, M. Peled, Y. Elbaz, E. Zahavi, H. Kasoto, Y. Reiter, A. Notcovich, and T. Bravman. 2010. Therapeutic antibodies: Discovery and development using the ProteOn XPR36 biosensor interaction array system. Anal Biochem 406: 147-156.
35. Oved, K., O. Ziv, J. Jacob-Hirsch, R. Noy, H. Novak, O. Makler, D. Galit, S. Keren, D. Segal, C. Gefen-Dor, N. Amariglio, G. Rechavi, and Y. Reiter. 2007. A novel postpriming regulatory check point of effector/memory T cells dictated through antigen density threshold-dependent anergy. J Immunol 178: 2307-2317.
36. Friedmann-Morvinski, D., A. Bendavid, T. Waks, D. Schindler, and Z. Eshhar. 2005. Redirected primary T cells harboring a chimeric receptor require costimulation for their antigen-specific activation. Blood 105: 3087-3093.
37. Eshhar, Z., T. Waks, A. Bendavid, and D. G. Schindler. 2001. Functional expression of chimeric receptor genes in human T cells. J Immunol Methods 248: 67-76.
38. Eshhar, Z., T. Waks, G. Gross, and D. G. Schindler. 1993. Specific activation and targeting of cytotoxic lymphocytes through chimeric single chains consisting of antibody-binding domains and the gamma or zeta subunits of the immunoglobulin and T-cell receptors. Proc Natl Acad Sci USA 90: 720-724.
39. Thomas, S., S. A. Xue, M. Cesco-Gaspere, E. San Jose, D. P. Hart, V. Wong, R. Debets, B. Alarcon, E. Morris, and H. J. Stauss. 2007. Targeting the Wilms tumor antigen 1 by TCR gene transfer: TCR variants improve tetramer binding but not the function of gene modified human T cells. J immunol 179: 5803-5810.
40. Kessels, H. W., M. C. Wolkers, M. D. van den Boom, M. A. van der Valk, and T. N. Schumacher. 2001. Immunotherapy through TCR gene transfer. Nat Immunol 2: 957-961.
41. Kantoff, P. W., C. S. Higano, N. D. Shore, E. R. Berger, E. J. Small, D. F. Penson, C. H. Redfern, A. C. Ferrari, R. Dreicer, R. B. Sims, Y. Xu, M. W. Frohlich, P. F. Schellhammer, and I. S. Investigators. 2010. Sipuleucel-T immunotherapy for castration-resistant prostate cancer. N Engl J Med 363: 411-422.
42. Dunn, S. M., P. J. Rizkallah, E. Baston, T. Mahon, B. Cameron, R. Moysey, F. Gao, M. Sami, J. Boulter, Y. Li, and B. K. Jakobsen. 2006. Directed evolution of human T cell receptor CDR2 residues by phage display dramatically enhances affinity for cognate peptide-MHC without increasing apparent cross-reactivity. Protein science: a publication of the Protein Sci 15: 710-721.
43. Li, Y., R. Moysey, P. E. Molloy, A. L. Vuidepot, T. Mahon, E. Baston, S. Dunn, N. Liddy, J. Jacob, B. K. Jakobsen, and J. M. Boulter. 2005. Directed evolution of human T-cell receptors with picomolar affinities by phage display. Nat biotechnol 23: 349-354.
44. Robbins, P. F., Y. F. Li, M. El-Gamil, Y. Zhao, J. A. Wargo, Z. Zheng, H. Xu, R. A. Morgan, S. A. Feldman, L. A. Johnson, A. D. Bennett, S. M. Dunn, T. M. Mahon, B. K. Jakobsen, and S. A. Rosenberg. 2008. Single and dual amino acid substitutions in TCR CDRs can enhance antigen-specific T cell functions. J Immunol 180: 6116-6131.
45. Reichert, J. M., and E. Dhimolea. 2012. The future of antibodies as cancer drugs. Drug Discov Today 17: 954-963.
46. May, C., P. Sapra, and H. P. Gerber. 2012. Advances in bispecific biotherapeutics for the treatment of cancer. Biochem Pharmacol 84: 1105-1112.
47. Holler, P. D., L. K. Chlewicki, and D. M. Kranz. 2003. TCRs with high affinity for foreign pMHC show self-reactivity. Nat Immunol 4: 55-62.
48. Thomas, S., S. A. Xue, C. R. Bangham, B. K. Jakobsen, E. C. Morris, and H. J. Stauss. 2011. Human T cells expressing affinity-matured TCR display accelerated responses but fail to recognize low density of MHC-peptide antigen. Blood 118: 319-329.
49. Schmid, D. A., M. B. Irving, V. Posevitz, M. Hebeisen, A. Posevitz-Fejfar, J. C. Sarria, R. Gomez-Eerland, M. Thome, T. N. Schumacher, P. Romero, D. E. Speiser, V. Zoete, O. Michielin, and N. Rufer. 2010. Evidence for a TCR affinity threshold delimiting maximal CD8 T cell function. J Immunol 184: 4936-4946.
50. Appay, V., D. C. Douek, and D. A. Price. 2008. CD8+ T cell efficacy in vaccination and disease. Nat Med 14: 623-628.

1. A chimeric antigen receptor (CAR) molecule comprising an extracellular domain comprising an antigen binding domain, a transmembrane domain, and an intracellular signaling domain, wherein an affinity of said binding domain to said antigen is characterized by a K_{D} higher than 150 nM.

2. The molecule of claim 1, wherein said antigen is an MHC restricted antigen.

3-4. (canceled)

5. The molecule of claim 1, wherein said antigen is a non-MHC restricted antigen.

6-13. (canceled)

14. The molecule of claim 1, wherein said antigen binding domain comprises a single chain Fv (scFv) molecule.

15-22. (canceled)

23. The molecule of claim 1, wherein said K_{D} is higher than 400 nM.

24. The molecule of claim 1, wherein said KD is selected from a range of about 200 nM (nanomolar) to about 5 μM (micromolar).

25. The molecule of claim 1, wherein said intracellular signaling domain comprises the polypeptide selected from the group consisting of: CD3ζ (CD247, CD3z), CD28, 4-1BB (CD137), ICOS, and OX40, and CD137.

26. An isolated polynucleotide comprising a nucleic acid sequence encoding the molecule of claim 1.

27. A nucleic acid construct comprising an isolated polynucleotide comprising a nucleic acid sequence encoding the molecule of claim 1 and a cis-acting regulatory element for directing transcription of said isolated polynucleotide in a host cell.

28. An isolated cell comprising the polynucleotide of claim 26.

29. The isolated cell of claim 28, wherein the cell is a T cell or natural killer (NK) cell.

30. The isolated cell of claim 29, wherein said T cell is obtained from peripheral blood mononuclear cells (PBMCs).

31. The isolated cell of claim 29, wherein said T cell comprises a Treg (T regulatory cell).

32. The isolated cell of claim 29, wherein said T cell comprises a CD4 T cell.

33. The isolated cell of claim 29, wherein said T cell comprises a CD8 T cell.

34. The isolated cell of claim 28, wherein said T cell is a cytotoxic T cell.

35. A pharmaceutical composition comprising the CAR molecule of the cell of claim 28 and a pharmaceutically acceptable carrier.

36. An in vitro method of generating a medicament for treating a pathology in a subject in need thereof, comprising:
(a) obtaining T cells or natural killer (NK) cells,
(b) transducing said T cells or said natural killer with the nucleic acid construct of claim 27, wherein binding of said molecule to said antigen elicits a therapeutic response by said T cells or said natural killer of the subject,
thereby generating the medicament for treating the pathology.

37. The in vitro method of claim 36, wherein said T cells or said natural killer are autologous to the subject or semi autologous to the subject.

38. (canceled)

39-40. (canceled)

41. A method of treating a pathology in a subject in need thereof, comprising administering said medicament resultant of claim 36 in the subject, thereby treating the pathology in the subject.

42-44. (canceled)
